(19) United States
(12) Patent Application Publication
(10) Pub. No.: US 2007/0190067 A1
(43) Pub. Date: Aug. 16, 2007

(54) Cationic Steroid Antimicrobial Compositions and Methods of Use

(76) Inventors: Paul B. Savage, Mapleton, UT (US); Derya Unutmaz, Nashville, TN (US)

Correspondence Address: PILLSBURY WINTHROP SHAW PITTMAN LLP, ATTENTION: DOCKETING DEPARTMENT, P.O BOX 10500, McLean, VA, 22102, US

(21) Appl. No.: 11/669,785

(22) Filed: Jan. 31, 2007

Related U.S. Application Data
(60) Provisional application No. 60/763,999, filed on Feb. 1, 2006.

(51) Int. Cl. A61K 39/42; C12Q 1/70; A61K 31/56

(52) U.S. Cl. 424/160.1; 514/171; 435/5

(57) ABSTRACT

The invention provides methods for decreasing or inhibiting human immunodeficiency virus (HIV) infection or pathogenesis (e.g., illness) of a cell in vitro, ex vivo or in vivo, a symptom or pathology associated with human immunodeficiency virus (HIV) infection or pathogenesis (e.g., illness) in vitro, ex vivo or in vivo, or an adverse side effect of human immunodeficiency virus (HIV) infection or pathogenesis (e.g., illness) in vitro, ex vivo or in vivo. In one embodiment, a method of the invention includes treating a subject with an invention compound (e.g., cationic steroid antimicrobial or CSA).

### RELATED APPLICATIONS

This application claims the benefit of priority of provisional application Ser. No. 60/763,999, filed Feb. 1, 2006, which is expressly incorporated herein by reference.

### GOVERNMENT FUNDING

Work described herein was supported in part by grants R01AI049131, awarded by the National Institutes of Health. The United States Government may have certain rights in this invention.

### TECHNICAL FIELD

The invention relates to methods of decreasing or inhibiting human immunodeficiency virus (HIV) infection or pathogenesis (e.g., illness) of a cell in vitro, ex vivo or in vivo, a symptom or pathology associated with human immunodeficiency virus (HIV) infection or pathogenesis (e.g., illness) in vitro, ex vivo or in vivo, or an adverse side effect of human immunodeficiency virus (HIV) infection or pathogenesis (e.g., illness) in vitro, ex vivo or in vivo. In one embodiment, a method of the invention includes treating a subject with an invention compound (e.g., cationic steroid antimicrobial or CSA).

### INTRODUCTION

HIV infection leads to a severe decrease in CD4(+) T lymphocytes, dysregulation of several leukocyte subpopulations and generalized immune activation, with the subsequent development of opportunistic infections and malignancies. Administration of highly active antiretroviral therapy (HAART) has been successful in reducing HIV plasma viremia; however, the ability of HAART to restore immunocompetence appears incomplete, particularly in patients with chronic and advanced disease. Development of alternative or complementary therapeutic approaches to HIV infection, particularly those able to compensate for the limitations of HAART, would be of interest.

### SUMMARY

Cationic steroid antimicrobials (CSAs) were developed as functional mimics of endogenous peptide antibiotics such as LL-37. A series of CSAs have been developed and CSAs are highly active against specific lipid-enveloped viruses including human immunodeficiency virus (HIV). Antiviral activities of multiple CSAs have been measured, and active and inactive forms have been identified.

### DESCRIPTION OF THE DRAWINGS

FIG. 1 is a drawing showing compounds of the invention.

FIG. 2 is a drawing showing compounds CSA-26 and CSA-46.

FIG. 3 is a drawing showing compound 134.

FIG. 4 is a drawing showing compound CSA-10.

FIG. 5 is a drawing showing compound 140.

FIG. 6 is a drawing showing compound CSA-31.

FIG. 7 is a drawing showing compounds 352-354.

FIG. 8 is a drawing showing compounds 341-343 and 324-327.

FIG. 9 is a drawing showing compounds 358.

FIG. 10 is a drawing showing various compounds of the invention (CSAs).

FIG. 11 is an ELISA study of HIV viral core protein p24, which is representative of four independent studies of HIV-VSV-G infection of cells.

FIG. 12 is a flow cytometry cell viability study of CSA's incubated with Hut cells (closed squares), activated primary CD4+ T cells (closed circles), HEK-293T cells (open squares) HeLa cells (open circles) and HIV.

FIG. 13 is a study of CSAs incubated with infectious HIV-VSV-G and Hut cells. Data are normalized to infection and are presented as the mean of three replicate samples from one representative study. GFP expression (closed squares) and flow cytometry of T cell viability (open squares). Error bars indicate standard deviation.

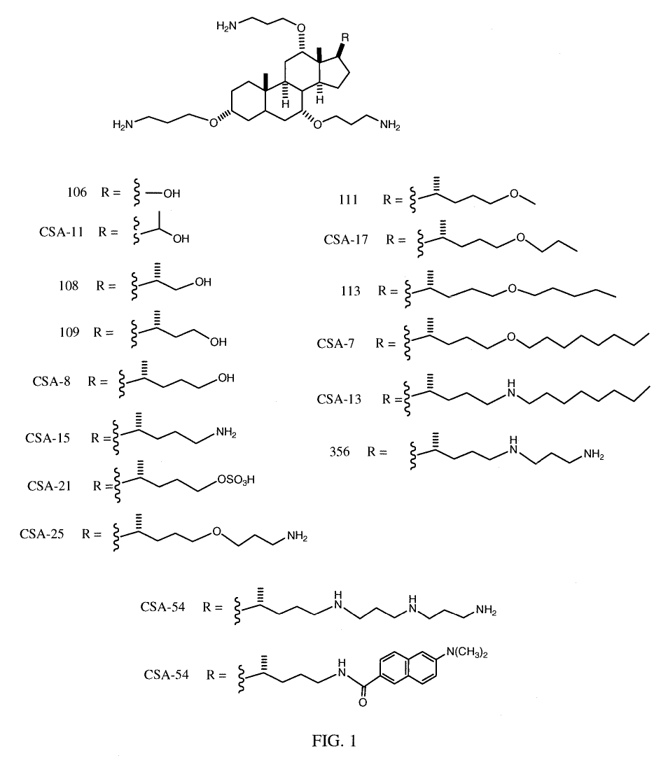

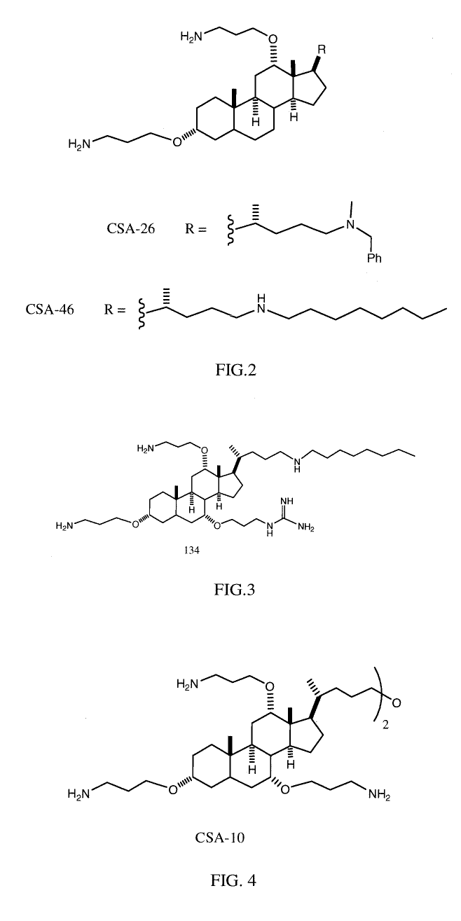

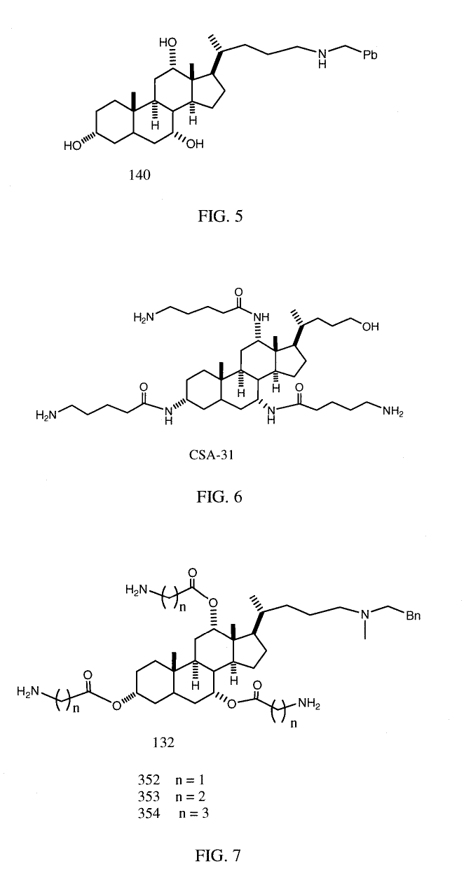

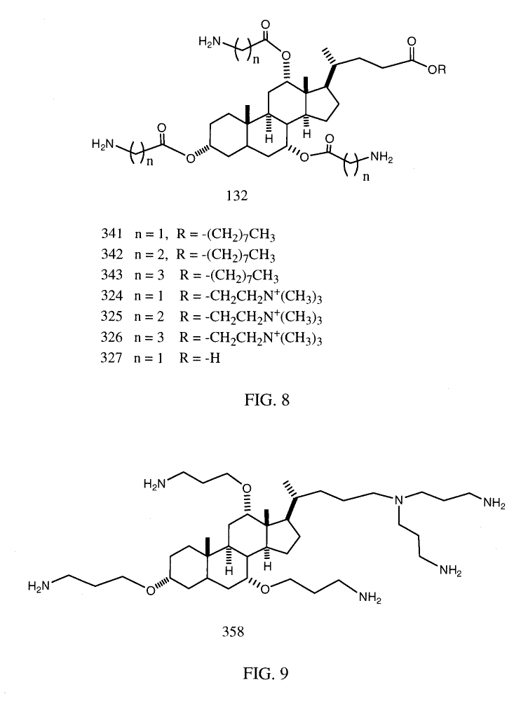

### DETAILED DESCRIPTION

In accordance with the invention, there are provided methods for decreasing or inhibiting human immunodeficiency virus (HIV) infection or pathogenesis (e.g., illness) of a cell in vitro, ex vivo or in vivo, a symptom or pathology associated with human immunodeficiency virus (HIV) infection or pathogenesis (e.g., illness) in vitro, ex vivo or in vivo, or an adverse side effect of human immunodeficiency virus (HIV) infection or pathogenesis (e.g., illness) in vitro, ex vivo or in vivo. In one embodiment, a method of the invention includes treating a subject with an invention compound (e.g., cationic steroid antimicrobial or CSA), wherein the subject is in need of treatment due to CSA anti-HIV activity or function, in order to provide the subject with a beneficial effect or improvement. In another embodiment, a method of the invention includes providing a subject with protection against an HIV infection or pathogenesis by administering a sufficient amount of cationic steroid antimicrobial (CSA) to provide the subject with protection against an HIV infection or pathogenesis. In a further embodiment, a method of the invention includes treating a subject for HIV infection or pathogenesis by administering a sufficient amount of cationic steroid antimicrobial (CSA) to treat the subject for the HIV infection or pathogenesis. In an additional embodiment, a method of the invention includes decreasing susceptibility of a subject to an HIV infection or pathogenesis by administering a composition comprising a sufficient amount of cationic steroid antimicrobial (CSA) to decrease susceptibility of the subject to an HIV infection or pathogenesis. Methods of the invention include administering CSA prior to, concurrently with, or following contact of the subject with or exposure of the subject to HIV; and administering CSA prior to, concurrently with, or following development of a symptom or pathology associated with or caused by HIV infection. In various aspects, a compound of the invention (e.g., CSA) is administered prior to (prophylaxis), concurrently with or following infection or exposure of the subject (therapeutic) to an HIV.

The invention treatment methods therefore include, among other things, therapeutic and prophylactic methods. Subjects can be contacted with, administered ex vivo or in vivo delivered a compound of the invention (e.g., CSA) prior to, concurrently with or following HIV exposure or contact, HIV infection, or development of a symptom or pathology associated with or caused by an HIV infection or pathogenesis.

The term “therapeutic” and grammatical variations thereof means the subject has an HIV infection, for example, the subject exhibits one or more symptoms or pathologies associated with or caused by HIV infection or pathogenesis (e.g., illness) as set forth herein or known in the art. The term “therapeutic” also includes a subject that has been exposed to or contacted with HIV but may not exhibit one or more symptoms or pathologies associated with or caused by HIV infection or pathogenesis (e.g., illness), as set forth herein or known in the art.

“Prophylaxis” and grammatical variations thereof refer to contact, administration or in vivo delivery to a subject prior to a known contact with or exposure to HIV. In situations where it is not known if a subject has been contacted with or exposed to HIV, contact with, administration or in vivo delivery of a compound to a subject occurs prior to manifestation or onset of a symptom associated with or caused by HIV infection or pathogenesis. In such a method, the effect of contact with, administration or in vivo delivery of a compound of the invention (e.g., CSA) can be to eliminate, prevent, inhibit, decrease or reduce the probability of or susceptibility towards developing an HIV infection or pathogenesis (e.g., illness), or a symptom or pathology associated with or caused by HIV infection or pathogenesis (e.g., illness).

As used herein, the term “associated with,” when used in reference to the relationship between a symptom, pathology or adverse side effect of HIV, means that the symptom, pathology or side effect is caused by HIV infection or pathogenesis, or is a secondary effect of the HIV infection or pathogenesis. A symptom, pathology or side effect that is present in a subject may therefore be the direct result of or caused by the HIV infection or pathogenesis (e.g., illness), or may be due at least in part to the subject reacting or responding to (e.g., an immunological response) HIV infection or pathogenesis (e.g., illness). For example, a symptom or pathology that occurs during an HIV infection or pathogenesis may be due in part to an inflammatory response of the subject.

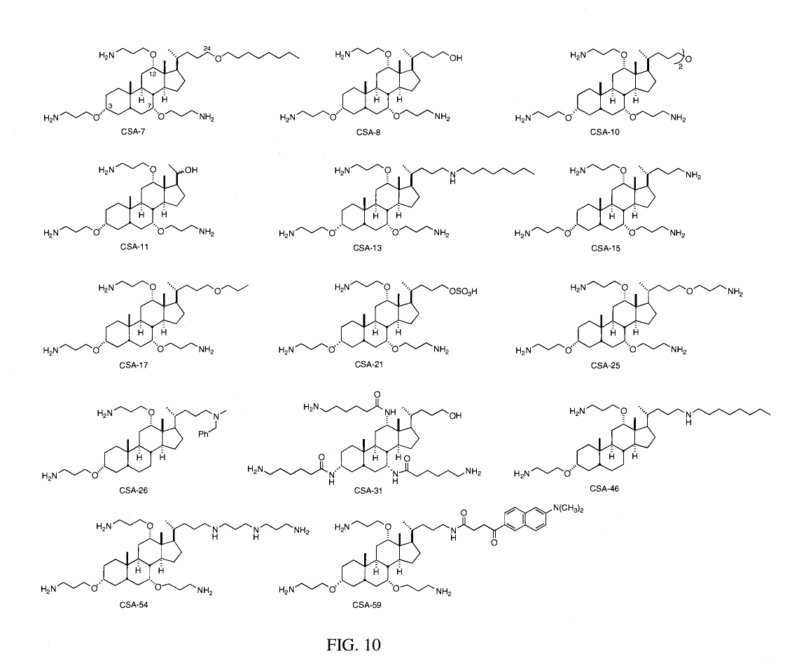

In particular embodiments of the compounds and methods of the invention, a CSA is selected from: CSA-7, CSA-8, CSA-10, CSA-11, CSA-13, CSA-15, CSA-17, CSA-21, CSA-25, CSA-26, CSA-31, CSA-46, CSA-54 and CSA-59, as set forth in FIG. 10. In other embodiments, a CSA does not have a charged group at position C24 or a CSA has a hydrophobic moiety at position C24 (e.g., a lipid). In additional embodiments, a CSA has a charged group at position C7. In further embodiments, a CSA comprises a multimer (e.g., a dimer, trimer, tetramer or higher order polymer). In yet additional embodiments, a CSA has a shorter tether length between the steroid scaffold and any amine group at positions C3, C7 or C12, relative to the tether length between the steroid scaffold and any amine group at positions C3, C7 or C12 of CSA-7, CSA-8, CSA-10, CSA-11, CSA-13, CSA-15, CSA-17, CSA-21, CSA-25, CSA-26, CSA-31, CSA-46, CSA-54 or CSA-59, as set forth in FIG. 10.

Methods of the invention, including, for example, prophylactic and therapeutic treatment methods, as well as methods for decreasing or preventing an adverse side effect of HIV, are applicable to HIV generally. HIV includes any strain or isolate or subtype or species of HIV, or combination of strains or isolates or subtypes or species of HIV. Particular examples are HIV−1 and HIV−2. Specific non-limiting examples of HIV−1 groups include Groups M, N and O. Additional examples are drug resistant HIV types, groups, subtypes or isolates. Specific non-limiting examples of HIV−1 subtypes include A, B, A/B, A/E, A/G, C, D, F, G, H, J and K subtypes, and mixtures thereof.

Methods of the invention include methods of treatment that results in a beneficial effect. Particular non-limiting examples of beneficial effects include providing a subject with partial or complete protection against an HIV infection or pathogenesis (e.g., illness), or a symptom caused by an HIV infection or pathogenesis (e.g., inhibit or reduce probability or susceptibility to an illness). Particular non-limiting examples of beneficial effects also include reducing, decreasing, inhibiting, delaying or preventing HIV infection or pathogenesis, and reducing, decreasing, inhibiting, ameliorating or preventing onset, severity, duration, progression, frequency or probability of one or more symptoms or pathologies associated with an HIV infection or pathogenesis. Additional non-limiting examples of beneficial effects also include reducing, decreasing, amounts of, or inhibiting, delaying or preventing increases in HIV titer or load, proliferation or replication. Further non-limiting particular examples of beneficial effects include reducing, decreasing, inhibiting, delaying, ameliorating or preventing onset, progression, severity, duration, frequency, probability or susceptibility of a subject to an HIV infection or pathogenesis (e.g., illness), or accelerating, facilitating or hastening recovery of a subject from an HIV infection or pathogenesis or one or more associated symptoms, pathologies or adverse side effects.

Methods of the invention therefore include providing a beneficial or therapeutic effect to a subject, for example, reducing, decreasing, inhibiting, delaying, ameliorating or preventing onset, progression, severity, duration, frequency or probability of HIV infection or pathogenesis or one or more symptoms or pathologies associated with or caused by HIV infection or pathogenesis; reducing, decreasing, inhibiting, delaying or preventing increases in HIV titer, viral load, replication, proliferation, or an amount of a viral protein of one or more HIV strains or isolates or subtypes. Stabilizing the infection, a symptom or pathology thereof, or preventing, inhibiting or delaying a worsening or progression of the infection or a symptom or pathology associated with or caused by HIV infection or pathogenesis, or progression of the underlying HIV infection, are also included in various embodiments of the methods of the invention.

Specific examples of symptoms and pathologies associated with or caused by HIV infection or pathogenesis (e.g., illness), whose onset, progression, severity, frequency, duration or probability can be reduced, decreased inhibited, delayed ameliorated or prevented include, for example, fever, fatigue, headache, sore throat, swollen lymph nodes, weight loss, diarrhea, rash, boils, warts, thrush, shingles, chronic or acute pelvic inflammatory disease (PID), dry cough, shortness of breath, bruising, bleeding, numbness or paralysis, muscle weakness, an opportunistic disorder, nerve damage, encephalopathy, dementia and death.

Specific examples of symptoms and pathologies associated with or caused by HIV infection or pathogenesis (e.g., illness), whose onset, progression, severity, frequency, duration or probability can be reduced, decreased inhibited, delayed ameliorated or prevented also include, for example, opportunistic disorders (e.g., bacterial, viral, fungal and parasitic infections). Non-limiting examples of opportunistic disorders include Candidiasis of bronchi, trachea, lungs or esophagus, cervical cancer, Coccidioidomycosis, Cryptococcosis, Cryptosporidiosis, Bacillary Angiomatosis, Cytomegalovirus (CMV), Cytomegalovirus retinitis, Herpes virus, Hepatitis virus, papilloma virus, Histoplasmosis, Isosporiasis, Kaposi's sarcoma, Burkitt's lymphoma, immunoblastic lymphoma, Mycobacterium avium, Mycobacterium tuberculosis, Pneuniocystis carinii, Pneumonia, progressive multifocal leukoencephalopathy (PML), Salmonelosis, Toxoplasmosis, Wasting syndrome and Lymphoid interstitial pneumonia/pulmonary lymphoid type. Other symptoms and pathologies of HIV infection or pathogenesis (e.g., illness), are known in the art and treatment thereof in accordance with the invention is provided.

An additional symptom that may be improved includes increasing numbers of CD4+ T cells, or stabilizing numbers of CD4+ T cells (e.g., greater than 500 or 200 cells/microliter blood). A further symptom that may be improved includes increasing the percentage of CD4+ T cells relative to other lymphocytes, or stabilizing the percentage of CD4+ T cells relative to other lymphocytes (e.g., greater than 15%).

Invention methods therefore also include increasing or stabilizing numbers of CD4+ T cells in an HIV+ subject. In one embodiment, a method includes administering a sufficient amount of CSA to increase or stabilize numbers of CD4+ T cells in the HIV+subject. In various aspects, CD4+ T cell counts less than 500 cells/microliter blood are increased or stabilized, CD4+ T cell counts less than 200 cells/microliter blood are increased or stabilized, or the percentage of CD4+ T cells less than 15% of all lymphocytes is increased or stabilized in the subject.

The methods of the invention, including, among other methods, providing a subject with protection against an HIV infection or pathogenesis, treatment of an HIV infection or pathogenesis, or a symptom or pathology associated with or caused by HIV infection or pathogenesis, or decreasing susceptibility of a subject to an HIV infection or pathogenesis, can therefore result in an improvement in the subjects' condition. An improvement is therefore any objective or subjective reduction, decrease, inhibition, delay, ameliorating or prevention of onset, progression, severity, duration, frequency or probability of one or more symptoms or pathologies associated with or caused by HIV infection or pathogenesis, or virus titer, viral load, replication, proliferation, or an amount of a viral protein. An improvement would also include reducing, inhibiting or preventing increases in virus titer, viral load, replication, proliferation, or an amount of a viral protein of one or more HIV strains or isolates or subtypes or species. An improvement would further include stabilizing a symptom or pathology associated with or caused by HIV infection or pathogenesis, or inhibiting, decreasing, delaying or preventing a worsening or progression of the symptom or pathology associated with or caused by HIV infection or pathogenesis, or progression of the underlying HIV infection. An improvement can therefore be, for example, in any of fever, fatigue, headache, sore throat, swollen lymph nodes, weight loss, diarrhea, rash, boils, warts, thrush, shingles, chronic or acute pelvic inflammatory disease (PID), dry cough, shortness of breath, bruising, bleeding, numbness or paralysis, muscle weakness, opportunistic disorders, nerve damage, encephalopathy, dementia, death, CD4+ T cell numbers or percentage of CD4+ T cell numbers relative to all lymphocytes, to any degree or for any duration of time (hours, days, weeks, months, years, or cure).

An improvement would also include reducing or eliminating a need, dosage amount or frequency of another treatment, such as an antiviral drug or other agent used for treating a subject having or at risk of having an HIV infection or pathogenesis or a symptom or pathology associated with or caused by HIV infection or pathogenesis. Thus, reducing an amount of another treatment for HIV infection or pathogenesis, a symptom or pathology associated with or caused by HIV, or an adverse side effect caused by HIV is considered to provide a benefit and, therefore, is considered within the invention methods. Non-limiting exemplary HIV treatments that may be eliminated or used at reduced doses or frequencies of administration include protease inhibitors, reverse transcriptase inhibitors, virus fusion inhibitors and virus entry inhibitors. Additional non-limiting exemplary HIV treatments include AK602, AMD070, APV, ATV, ATZ, AVX754, AZT, Abacavir, Acyclovir, Adefovir dipivoxil, Adriamycin, Agenerase, Aldesleukin, Alovudine, AmBisome, Amdoxovir, Amphocin, Amphotec, Amphotericin B, Ampligen, Amprenavir, Androderm, Androgel, Aptivus, Atazanavir, Azithromycin, BMS-488043, Bactrim, Baraclude, Biaxin, BufferGel, C31G, CD4-IgG2, CPV, CS, Calanolide A, Capravirine, Carbopol 974P, Carrageenan, Carraguard, Cellulose sulfate, Clarithromycin, Combivir, Copegus, Cotrimoxazole, Crixivan, Cyanovirin-N, Cytovene, DAPD, DLV, DPC 817, DS, Delavirdine, Depo-Testosterone, Dextran sulfate, Didanosine, Diflucan, Doxil, Doxorubicin, Dronabinol, EFV, Efavirenz, Elvucitabine, Emtricitabine, Emtriva, Enfuvirtide, Entecavir, Epivir, Epoetin alfa, Epogen, Epzicom, Etopophos (phosphate salt), Etoposide, Etravirine, Fluconazole, Fortovase, Fosamprenavir, Fungizone, Fuzeon, GSK-873, 140 (aplaviroc), GW433908, Gammar-P, Ganciclovir, Growth hormone, Human growth hormone, HEC, Hepsera, Hivid, Hydroxyethyl cellulose, IDV, IGIV, Interleukin-2 (IL-2), INH, Immune Globulin, Indinavir, Interferon alfa-2, Intron A (2b), Invirase, Isoniazid, Itraconazole, KP-1461, Kaletra, L-000870810, LPV/RTV, Lamivudine, Lexiva, Marinol, Megace, Megestrol, Mycobutin, NFV, NVP, Naphthalene 2-sulfonate polymer, Nebupent, Nelfinavir, Neutrexin, Nevirapine, New-Fill, Norvir, Nydrazid, Onxol, PA-457, PMPA, PRO2000, PRO542, Paclitaxel, Paxene, Pegasys (2a), Pentamidine, Peptide T, Poly(I)-Poly(C12U), Poly-L-lactic acid, Polygam S/D, Procrit, Proleukin, RCV, RTV, RVT, Racivir, Rebetol, Rescriptor, Retrovir, Reverset, Reyataz, Ribavirin, Rifabutin, Rifadin, Rifampin, Rimactane, Ritonavir, Roferon-A (2a), SCH-C, SCH-D (vicriviroc), SQV, Saquinavir, Savvy, Sculptra, Septra, Serostim, Somatropin, Sporanox, Stavudine, Sulfamethoxazole, Sustanon, Sustiva, T-20, TDF, THC, TMC114, TMC125, TNX-355, Taxol, Tenofovir, Tenofovir disoproxil fumarate, Testosterone, Tipranavir, Toposar, Trimethoprim, Trimetrexate, Trizivir, Truvada, UC-781, UK-427,857 (maraviroc), Ushercell, Valcyte, Valganciclovir, Valproic acid, VePesid, Vicriviroc, Videx, Viracept, Viramune, Virazole, Viread, Vitrasert, ZDV, Zalcitabine, Zerit, Ziagen, Zidovudine, Zithromax, Zovirax, D4T, ddC, β-LFddC, P-LFd4C, DDI, f-APV, 3TC, and human erythropoietin (EPO). Still additional non-limiting exemplary HIV treatments include cytokines, chemokines, interferons and interleukins. Further non-limiting exemplary HIV treatments vaccination with or against HIV or an HIV protein, and an antibody that binds to an HIV protein (e.g., envelope protein gp160, gp120 or gp41, gag protein, pol protein, p7, p17, p24, tat, rev, nef, vif, vpr, vpu, reverse transcriptase, integrase, or protease.

A treatment or improvement need not be complete ablation of any particular infection, pathogenesis (e.g., illness), symptom, pathology or adverse side effect, or all of the infection, pathology, symptoms, pathologies or adverse side effects associated with or caused by HIV infection or pathogenesis (e.g., illness), or vaccination against an HIV. Rather, treatment may be any objective or subjective measurable or detectable anti-virus effect or improvement in a treated subject. Thus, reducing, inhibiting decreasing, eliminating, delaying, halting or preventing a progression or worsening of the infection or pathogenesis (e.g., illness), a symptom or pathology of the infection or pathogenesis (e.g., illness), or an adverse side effect caused by vaccination is a satisfactory outcome. For example, a compound of the invention (e.g., CSA) may reduce, delay or stabilize fever, but not have any effect on fever, fatigue, headache, sore throat, swollen lymph nodes, weight loss, diarrhea, rash, boils, warts, thrush, shingles, chronic or acute pelvic inflammatory disease (PID), dry cough, shortness of breath, bruising, bleeding, numbness or paralysis, muscle weakness, opportunistic disorders, nerve damage, encephalopathy, dementia and death. Another example is where a compound of the invention reduces fatigue and headache, without a detectable improvement in one or more other symptoms or pathologies. Thus, a satisfactory clinical endpoint is achieved when there is an incremental improvement in the subject's condition or a partial reduction or a stabilization of an HIV infection, pathogenesis (e.g., illness) or a symptom, pathology or adverse side effect thereof, or an inhibition or prevention of worsening or progression of the HIV infection, pathogenesis, symptom, pathology or adverse side effect thereof (stabilizing one or more symptoms or pathologies), over a short or long duration (hours, days, weeks, months, years, or cure).

In the methods of the invention in which there is a desired outcome, for example, a therapeutic or prophylactic method that provides an objective or subjective improvement in an HIV infection or pathogenesis (e.g., illness), a symptom or pathology associated with or caused by HIV, or an adverse side effect caused by HIV, a compound of the invention (e.g., CSA) can be administered in a sufficient or effective amount. As used herein, a “sufficient amount” or “effective amount” or an “amount sufficient” or an “amount effective” refers to an amount that provides, in single or multiple doses, alone or in combination with one or more other compounds, treatments, agents (e.g., a drug) or therapeutic regimens, a long term or a short term detectable or measurable improvement or beneficial effect to a given subject of any degree or for any time period or duration (e.g., for minutes, hours, days, months, years, or cured).

A “sufficient amount” or “effective amount” therefore includes decreasing, reducing, inhibiting, preventing, or delaying onset; decreasing, reducing, inhibiting, delaying, or preventing a progression or worsening of; or reducing, relieving, ameliorating, or alleviating, severity, frequency, duration, susceptibility or probability of HIV infection or pathogenesis (e.g., illness), one or more symptoms associated with or caused by HIV infection or pathogenesis (e.g., illness), or an adverse side effect of HIV. In addition, hastening a subject's recovery from HIV infection or pathogenesis, one or more symptoms associated with or caused by HIV infection or pathogenesis, or an adverse side effect of HIV is considered to be a sufficient or effective amount. Various beneficial effects and indicia of therapeutic and prophylactic benefit are as set forth herein and are known to the skilled artisan.

A sufficient amount or an effective amount can but need not be provided in a single administration and can but need not be administered alone (i.e., without a second drug, agent, treatment or therapeutic regimen), or in combination with another compound, agent, treatment or therapeutic regimen. In addition, a sufficient amount or an effective amount need not be sufficient or effective if given in single or multiple doses without a second compound, treatment, agent, or therapeutic regimen, since additional doses, amounts, frequency or duration of administration above and beyond such doses, or additional compounds, agents, treatments or therapeutic regimens may be included in order to be effective or sufficient in a given subject.

A sufficient amount or an effective amount need not be effective in each and every subject, nor a majority of subjects in a given group or population. Thus, a sufficient amount or an effective amount means sufficiency or effectiveness in a particular subject, not a group or the general population. As is typical for such methods, some subjects will exhibit a greater or less response to a method of the invention than other subjects.

Amounts, frequencies or duration also considered sufficient and effective and are therefore beneficial are those that result in the elimination or a reduction in amount, frequency or duration of another compound, agent, treatment or therapeutic regimen. For example, a compound of the invention is considered as having a beneficial or therapeutic effect if contact, administration or delivery in vivo results in the use of a lesser amount, frequency or duration of another compound, agent, treatment or therapeutic regimen to treat the infection, pathogenesis, symptom or pathology, or adverse side effect.

Any compound, agent, treatment or other therapeutic regimen having a beneficial, additive, synergistic or complementary activity or effect can be formulated or used in combination with or in addition to the invention compounds (e.g., CSAs). In various embodiments, the compound, agent, treatment or therapeutic regimen is for providing a subject with protection against an HIV infection or pathogenesis (e.g., illness); treating a subject for HIV infection or pathogenesis (e.g., illness); decreasing susceptibility of a subject to an HIV infection or pathogenesis (e.g., illness); treating an opportunistic disorder caused by or associated with HIV infection or pathogenesis; or decreasing or preventing an adverse side effect caused by HIV infection or pathogenesis or an HIV treatment. Thus, compositions of the invention include CSA combinations with other CSAs, CSA combinations with other agents or treatments (e.g., anti-HIV drugs, such as protease inhibitors, reverse transcriptase inhibitors, virus fusion inhibitors and virus entry inhibitors, live or attenuated HIV, HIV proteins, HIV antibodies, etc.), and methods of the invention include contact with, administration in vitro or in vivo, with another compound (e.g., another CSA), agent, treatment or therapeutic regimen appropriate for the condition to be treated. The compound (e.g., another CSA), agent, treatment or therapeutic regimen appropriate may be used in accordance with the prophylactic and therapeutic treatment methods, as well as methods for treating an opportunistic disorder caused by or associated with HIV infection or pathogenesis, or decreasing or preventing an adverse side effect caused by HIV infection or pathogenesis or an HIV treatment, as set forth herein, prior to, concurrently or following contacting or administering a compound of the invention (e.g., CSA) in vitro or in vivo.

Examples of such combination compositions and methods include protease inhibitors, reverse transcriptase inhibitors, virus fusion inhibitors and virus entry inhibitors, live or attenuated HIV, HIV proteins and antibodies that bind to HIV proteins. A pool of protease inhibitors, reverse transcriptase inhibitors, virus fusion inhibitors and virus entry inhibitors, live or attenuated HIV, HIV proteins or HIV binding antibodies (e.g., monoclonal or polyclonal) can be combined with a compound of the invention or administered separately (prior to, concurrently with or following) administration of a compound in accordance with the invention. Additional examples of combination compositions and methods include HIV and other treatments such as AK602, AMD070, APV, ATV, ATZ, AVX754, AZT, Abacavir, Acyclovir, Adefovir dipivoxil, Adriamycin, Agenerase, Aldesleukin, Alovudine, AmBisome, Amdoxovir, Amphocin, Amphotec, Amphotericin B, Ampligen, Amprenavir, Androderm, Androgel, Aptivus, Atazanavir, Azithromycin, BMS-488043, Bactrim, Baraclude, Biaxin, BufferGel, C31G, CD4-IgG2, CPV, CS, Calanolide A, Capravirine, Carbopol 974P, Carrageenan, Carraguard, Cellulose sulfate, Clarithromycin, Combivir, Copegus, Cotrimoxazole, Crixivan, Cyanovirin-N, Cytovene, DAPD, DLV, DPC 817, DS, Delavirdine, Depo-Testosterone, Dextran sulfate, Didanosine, Diflucan, Doxil, Doxorubicin, Dronabinol, EFV, Efavirenz, Elvucitabine, Emtricitabine, Emtriva, Enfuvirtide, Entecavir, Epivir, Epoetin alfa, Epogen, Epzicom, Etopophos (phosphate salt), Etoposide, Etravirine, Fluconazole, Fortovase, Fosamprenavir, Fungizone, Fuzeon, GSK-873,140 (aplaviroc), GW433908, Gammar-P, Ganciclovir, Growth hormone, Human growth hormone, HEC, Hepsera, Hivid, Hydroxyethyl cellulose, IDV, IGIV, Interleukin-2 (IL-2), INH, Immune Globulin, Indinavir, Interferon alfa-2, Intron A (2b), Invirase, Isoniazid, Itraconazole, KP-1461, Kaletra, L-000870810, LPV/RTV, Lamivudine, Lexiva, Marinol, Megace, Megestrol, Mycobutin, NFV, NVP, Naphthalene 2-sulfonate polymer, Nebupent, Nelfinavir, Neutrexin, Nevirapine, New-Fill, Norvir, Nydrazid, Onxol, PA-457, PMPA, PRO2000, PRO542, Paclitaxel, Paxene, Pegasys (2a), Pentamidine, Peptide T, Poly(I)-Poly(C12U), Poly-L-lactic acid, Polygam S/D, Procrit, Proleukin, RCV, RTV, RVT, Racivir, Rebetol, Rescriptor, Retrovir, Reverset, Reyataz, Ribavirin, Rifabutin, Rifadin, Rifampin, Rimactane, Ritonavir, Roferon-A (2a), SCH-C, SCH-D (vicriviroc), SQV, Saquinavir, Savvy, Sculptra, Septra, Serostim, Somatropin, Sporanox, Stavudine, Sulfamethoxazole, Sustanon, Sustiva, T-20, TDF, THC, TMC114, TMC125, TNX-355, Taxol, Tenofovir, Tenofovir disoproxil fumarate, Testosterone, Tipranavir, Toposar, Trimethoprim, Trimetrexate, Trizivir, Truvada, UC-781, UK-427,857 (maraviroc), Ushercell, Valcyte, Valganciclovir, Valproic acid, VePesid, Vicriviroc, Videx, Viracept, Viramune, Virazole, Viread, Vitrasert, ZDV, Zalcitabine, Zerit, Ziagen, Zidovudine, Zithromax, Zovirax, D4T, ddC, β-LFddC, P-LFd4C, DDI, f-APV, 3TC, and human erythropoietin (EPO). Still additional non-limiting exemplary HIV and other treatments include cytokines, chemokines, interferons and interleukins.

Further additional exemplary HIV and other treatments include with an HIV protein (e.g., present on one or more of HIV−1 or HIV−2, such as envelope protein gp160, gp120 or gp41, gag protein, pol protein, p7, p17, p24, tat, rev, nef, vif, vpr, vpu, reverse transcriptase, integrase, or protease), an antibody that binds to an HIV protein (e.g., present on one or more of HIV−1 or HIV−2, such as envelope protein gp160, gp120 or gp41, gag protein, pol protein, p7, p17, p24, tat, rev, nef, vif, vpr, vpu, reverse transcriptase, integrase, or protease). HIV proteins and binding antibodies include those present on or that bind to one or more of HIV−1 (e.g., Groups M, N and O, or subtypes include A, B, A/B, A/E, A/G, C, D, F, G, H, J and K subtypes, and mixtures thereof) or HIV−2, drug resistant HIV types, groups, subtypes or isolates.

Still additional examples of combination compositions and methods include immune system enhancing and anti-cell proliferative treatments (tumors or cancers). Specific non-limiting examples include cytokines, chemokines, interferons, interleukins, internal or external radiotherapy, surgical resection, hyperthermia, and chemotherapeutic agents.

Antibodies include proteins that bind to other molecules (antigens) via heavy and light chain variable domains, V_{H} and V_{L}, respectively. An antibody is any polyclonal or monoclonal immunoglobulin molecule, or mixture thereof, such as IgM, IgG, IgA, IgE, IgD, and any subclass thereof, such as IgG₁, IgG₂, IgG₃, IgG₄, etc. A monoclonal antibody, refers to an antibody that is based upon, obtained from or derived from a single clone, including any eukaryotic, prokaryotic, or phage clone. An antibody also includes a functional (e.g., binding) fragment or subsequence, such as, for example, Fab, Fab′, F(ab′)₂, Fv, Fd, scFv and sdFv, unless otherwise expressly stated.

Antibodies include those specific or selective for binding to HIV protein or a homolog. That is, binding to proteins other than the HIV protein or a homolog is such that the binding does not significantly interfere with detection of the HIV protein or homolog, unless such other proteins have a similar or same epitope the HIV protein or homolog that is recognized by the HIV antibody. Selective binding can be distinguished from non-selective binding using specificity, affinity and other binding assays, competitive and non-competitive, known in the art.

Antibodies include “human” forms, which mean that the amino acid sequence of the antibody is fully human or can or do exist in a human antibody. An antibody that is non-human may be made fully human by substituting non-human amino acid residues with amino acid residues that can or do exist in a human antibody. Amino acid residues present in human antibodies, CDR region maps and human antibody consensus residues are known in the art (see, e.g., Kabat, Sequences of Proteins of Immunological Interest, 4^{th} Ed. US Department of Health and Human Services. Public Health Service (1987); Chothia and Lesk J. Mol. Biol. 186:651 (1987); Padlan Mol. Immunol. 31:169 (1994); and Padlan Mol. Immunol. 28:489 (1991)).

Antibodies include “human” forms, which means that the amino acid sequence of the antibody has non-human amino acid residues (e.g., mouse, rat, goat, rabbit, etc.) of one or more complementarity determining regions (CDRs) that specifically bind to the desired antigen in an acceptor human immunoglobulin molecule, and one or more human amino acid residues in the Fv framework region (FR), which are amino acid residues that flank the CDRs. Antibodies referred to as “primatized” in the art are within the meaning of “humanized” as used herein, except that the acceptor human immunoglobulin molecule and framework region amino acid residues may be any primate amino acid residue (e.g., ape, gibbon, gorilla, chimpanzees orangutan, macaque), in addition to any human residue.

Antibodies include “chimeric” forms, which means that the amino acid sequence of the antibody contains one or more portions that are derived from, obtained or isolated from, or based upon two or more different species. That is, for example, a portion of the antibody may be human (e.g., a constant region) and another portion of the antibody may be non-human (e.g., a murine heavy or light chain variable region). Thus, a chimeric antibody is a molecule in which different portions of the antibody are of different species origins. Unlike a humanized antibody, a chimeric antibody can have the different species sequences in any region of the antibody.

The term “subject” refers to an animal, typically mammalian animals, such as but not limited to non-human primates (apes, gibbons, gorillas, chimpanzees, orangutans, macaques), domestic animals (dogs and cats), a farm animals (chickens, ducks, horses, cows, goats, sheep, pigs), experimental animal (mouse, rat, rabbit, guinea pig) and humans. Subjects include animal models, for example, a model of HIV infection (e.g., a primate SIV model). Subjects include naturally occurring or non-naturally occurring mutated or non-human genetically engineered (e.g., transgenic or knockout) animals. Subjects further include animals having or at risk of having a chronic or acute HIV infection or pathogenesis, symptom of HIV infection or pathogenesis, or adverse side effect caused by HIV. Subjects can be any age. For example, a subject (e.g., human) can be a newborn, infant, toddler, child, teenager, or adult, e.g., 50 years or older.

Subjects include those in need of a method of the invention, e.g., in need of a therapeutic or prophylactic treatment. A subject is considered to be in need of a method of the invention where a method is likely to provide some benefit to a subject. Various benefits provided to a subject are as set forth herein and known in the art for HIV infection, pathogenesis (e.g., illness), symptoms or pathologies caused by or associated with HIV infection or pathogenesis (e.g., illness), and adverse side effects caused by HIV.

Subjects appropriate for treatment include those having HIV infection or pathogenesis or having any symptom or pathology associated with or caused by HIV. Target subjects therefore include subjects that have been infected with HIV, have been diagnosed as HIV+, or that have developed one or more adverse symptoms or pathologies associated with or caused by HIV infection or pathogenesis (e.g., illness), regardless of the virus type, timing or degree of onset, progression, severity, frequency, duration of any infection, pathogenesis (e.g., illness), symptom, pathology or adverse side effect. Subjects further include subjects those having reduced numbers of CD4+ T cells, as compared to an age, gender, race, etc. matched subject. For example, a subject in need of treatment would include those HIV+ and having a CD4+ T cell count less than 500 cells/microliter blood, or less than 200 cells/microliter blood, or the percentage of CD4+ T cells in the subject is less than 15% of all lymphocytes.

Subjects appropriate for treatment also include those at risk of HIV infection or pathogenesis or at risk of having or developing an HIV infection. Candidate subjects therefore include subjects that have been exposed to or contacted with HIV, or that are at risk of exposure to or contact with HIV, regardless of the type, timing or extent of exposure or contact. The invention methods are therefore applicable to a subject who is at risk of HIV infection or pathogenesis, but has not yet been exposed to or contacted with HIV. Prophylactic methods are therefore included. Subjects targeted for prophylaxis can be at increased risk (probability or susceptibility) of HIV infection or pathogenesis, as set forth herein and known in the art.

At risk subjects appropriate for treatment include subjects exposed to other subjects having HIV, or where the risk of HIV infection is increased due to changes in virus infectivity or cell tropism, immunological susceptibility (e.g., an immunocompromised subject), or environmental risk. At risk subjects appropriate for treatment therefore include human subjects exposed to or at risk of exposure to other humans that have an HIV infection (e.g., diagnosed as HIV+)

Subjects also appropriate for treatment also include those vaccinated against or a candidate for vaccination against HIV (e.g., vaccinated with live or attenuated HIV, HIV protein or antibody that binds to HIV protein). Subjects therefore include vaccinated subjects that have not or have been exposed to or contacted with HIV, as well as candidate subjects for vaccination that have not or have been exposed to or contacted with HIV, regardless of the type, timing or extent of exposure or contact.

In various embodiments, a subject has or is a candidate for vaccination against HIV (e.g., vaccinated with live or attenuated HIV, HIV protein or antibody that binds to HIV protein). In various aspects, a subject is administered a compound of the invention (e.g., CSA) prior to, concurrently with, or following vaccination against HIV (e.g., within 0-2, 2-4, 4-12 or 12-24 hours or days of vaccination).

Subjects further include immunocompromised subjects due to an immunological disorder (e.g., autoimmunity) or disease, or an immune-suppressing treatment (e.g., cyclophosphamide). Subjects also include those having been exposed to HIV or diagnosed as HIV+. Subjects further include those receiving or candidates for a tissue or organ transplant.

Compounds of the invention, including CSAs, can be incorporated into pharmaceutical compositions or formulations. Such pharmaceutical compositions/formulations are useful for administration to a subject, in vivo or ex vivo.

Pharmaceutical compositions and formulations include carriers or excipients for administration to a subject. As used herein the terms “pharmaceutically acceptable” and “physiologically acceptable” mean a biologically compatible formulation, gaseous, liquid or solid, or mixture thereof, which is suitable for one or more routes of administration, in vivo delivery or contact. A formulation is compatible in that it does not destroy activity of an active ingredient therein (e.g., a CSA), or induce adverse side effects that far outweigh any prophylactic or therapeutic effect or benefit.

Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

The formulations may, for convenience, be prepared or provided as a unit dosage form. Preparation techniques include bringing into association the active ingredient (e.g., CSA) and a pharmaceutical carrier(s) or excipient(s). In general, formulations are prepared by uniformly and intimately associating the active ingredient with liquid carriers or finely divided solid carriers or both, and then, if necessary, shaping the product. For example, a tablet may be made by compression or molding. Compressed tablets may be prepared by compressing, in a suitable machine, an active ingredient (e.g., a CSA) in a free-flowing form such as a powder or granules, optionally mixed with a binder, lubricant, inert diluent, preservative, surface-active or dispersing agent. Molded tablets may be produced by molding, in a suitable apparatus, a mixture of powdered compound (e.g., CSA) moistened with an inert liquid diluent. The tablets may optionally be coated or scored and may be formulated so as to provide a slow or controlled release of the active ingredient therein.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

Supplementary active compounds (e.g., preservatives, antioxidants, antimicrobial agents including biocides and biostats such as antibacterial, antiviral and antifungal agents) can also be incorporated into the compositions. Preservatives and other additives include, for example, antimicrobials, anti-oxidants, chelating agents and inert gases (e.g., nitrogen). Pharmaceutical compositions may therefore include preservatives, antimicrobial agents, anti-oxidants, chelating agents and inert gases.

Preservatives can be used to inhibit microbial growth or increase stability of the active ingredient thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

An antimicrobial agent or compound directly or indirectly inhibits, reduces, delays, halts, eliminates, arrests, suppresses or prevents contamination by or growth, infectivity, replication, proliferation, reproduction, of a pathogenic or non-pathogenic microbial organism. Classes of antimicrobials include, antibacterial, antiviral, antifungal and antiparasitics. Antimicrobials include agents and compounds that kill or destroy (-cidal) or inhibit (-static) contamination by or growth, infectivity, replication, proliferation, reproduction of the microbial organism.

Exemplary antibacterials (antibiotics) include penicillins (e.g., penicillin G, ampicillin, methicillin, oxacillin, and amoxicillin), cephalosporins (e.g., cefadroxil, ceforanid, cefotaxime, and ceftriaxone), tetracyclines (e.g., doxycycline, chlortetracycline, minocycline, and tetracycline), aminoglycosides (e.g., amikacin, gentamycin, kanamycin, neomycin, streptomycin, netilmicin, paromomycin and tobramycin), macrolides (e.g., azithromycin, clarithromycin, and erythromycin), fluoroquinolones (e.g., ciprofloxacin, lomefloxacin, and norfloxacin), and other antibiotics including chloramphenicol, clindamycin, cycloserine, isoniazid, rifampin, vancomycin, aztreonam, clavulanic acid, imipenem, polymyxin, bacitracin, amphotericin and nystatin.

Particular non-limiting classes of anti-virals include reverse transcriptase inhibitors; protease inhibitors; thymidine kinase inhibitors; sugar or glycoprotein synthesis inhibitors; structural protein synthesis inhibitors; nucleoside analogues; and viral maturation inhibitors. Specific non-limiting examples of anti-virals include those set forth above and, nevirapine, delavirdine, efavirenz, saquinavir, ritonavir, indinavir, nelfinavir, amprenavir, zidovudine (AZT), stavudine (d4T), larnivudine (3TC), didanosine (DDI), zalcitabine (ddC), abacavir, acyclovir, penciclovir, valacyclovir, ganciclovir, 1,-D-ribofuranosyl-1,2,4-triazole-3 carboxamide, 9->2-hydroxy-ethoxy methylguanine, adamantanamine, 5-iodo-2′-deoxyuridine, trifluorothymidine, interferon and adenine arabinoside.

Exemplary antifungals include agents such as benzoic acid, undecylenic alkanolamide, ciclopiroxolamine, polyenes, imidazoles, allylamine, thiocarbamates, amphotericin B, butylparaben, clindamycin, econaxole, amrolfine, butenafine, naftifine, terbinafine, ketoconazole, elubiol, econazole, econaxole, itraconazole, isoconazole, miconazole, sulconazole, clotrimazole, enilconazole, oxiconazole, tioconazole, terconazole, butoconazole, thiabendazole, voriconazole, saperconazole, sertaconazole, fenticonazole, posaconazole, bifonazole, fluconazole, flutrimazole, nystatin, pimaricin, amphotericin B, flucytosine, natamycin, tolnaftate, mafenide, dapsone, caspofungin, actofunicone, griseofulvin, potassium iodide, Gentian Violet, ciclopirox, ciclopirox olamine, haloprogin, ketoconazole, undecylenate, silver sulfadiazine, undecylenic acid, undecylenic alkanolamide and Carbol-Fuchsin.

Pharmaceutical compositions can optionally be formulated to be compatible with a particular route of administration. Thus, pharmaceutical compositions include carriers (excipients, diluents, vehicles or filling agents) suitable for administration by various routes and delivery, locally, regionally or systemically.

Exemplary routes of administration for contact or in vivo delivery which a compound of the invention (e.g., CSA) can optionally be formulated include inhalation, respiration, intubation, intrapulmonary instillation, oral (buccal, sublingual, mucosal), intrapulmonary, rectal, vaginal, intrauterine, intradermal, topical, dermal, parenteral (e.g., subcutaneous, intramuscular, intravenous, intradermal, intraocular, intratracheal and epidural), intranasal, intrathecal, intraarticular, intracavity, transdermal, iontophoretic, ophthalmic, optical (e.g., corneal), intraglandular, intraorgan, intralymphatic.

Formulations suitable for parenteral administration include aqueous and non-aqueous solutions, suspensions or emulsions of the compound, which may include suspending agents and thickening agents, which preparations are typically sterile and can be isotonic with the blood of the intended recipient. Non-limiting illustrative examples of aqueous carriers include water, saline (sodium chloride solution), dextrose (e.g., Ringer's dextrose), lactated Ringer's, fructose, ethanol, animal, vegetable or synthestic oils. Examples of non-aqueous solvents are propylene glycol, polyethylene glycol, vegetable oils such as olive oil, and injectable organic esters such as ethyl oleate. Intravenous vehicles include fluid and nutrient replenishers, electrolyte replenishers (such as those based on Ringer's dextrose). The formulations may be presented in unit-dose or multi-dose kits, for example, ampules and vials, and may be stored in a freeze-dried (lyophilized) condition requiring addition of a sterile liquid carrier, for example, water for injections, prior to use.

For transmucosal or transdermal administration (e.g., topical contact), penetrants can be included in the pharmaceutical composition. Penetrants are known in the art, and include, for example, for transmucosal administration, detergents, bile salts, and fusidic acid derivatives. For transdermal administration, the active ingredient can be formulated into aerosols, sprays, ointments, salves, gels, pastes, lotions, oils or creams as generally known in the art.

For topical administration, for example, to skin, pharmaceutical compositions typically include ointments, creams, lotions, pastes, gels, sprays, aerosols or oils. Carriers which may be used include Vaseline, lanolin, polyethylene glycols, alcohols, transdermal enhancers, and combinations thereof. An exemplary topical delivery system is a transdermal patch containing an active ingredient (e.g., CSA).

For oral administration, pharmaceutical compositions include capsules, cachets, lozenges, tablets or troches, as powder or granules. Oral administration formulations also include a solution or a suspension (e.g., aqueous liquid or a non-aqueous liquid; or as an oil-in-water liquid emulsion or a water-in-oil emulsion).

For airway or nasal administration, pharmaceutical compositions can be formulated in a dry powder for delivery, such as a fine or a coarse powder having a particle size, for example, in the range of 20 to 500 microns which is administered in the manner by inhalation through the airways or nasal passage. Depending on delivery device efficiency, effective dry powder dosage levels typically fall in the range of about 10 to about 100 mg. Appropriate formulations, wherein the carrier is a liquid, for administration, as for example, a nasal spray or as nasal drops, include aqueous or oily solutions of the active ingredient.

For airway or nasal administration, aerosol and spray delivery systems and devices, also referred to as “aerosol generators” and “spray generators,” such as metered dose inhalers (MDI), nebulizers (ultrasonic, electronic and other nebulizers), nasal sprayers and dry powder inhalers can be used. MDIs typically include an actuator, a metering valve, and a container that holds a suspension or solution, propellant, and surfactant (e.g., oleic acid, sorbitan trioleate, lecithin). Activation of the actuator causes a predetermined amount to be dispensed from the container in the form of an aerosol, which is inhaled by the subject. MDIs typically use liquid propellant and typically, MDIs create droplets that are 15 to 30 microns in diameter, optimized to deliver doses of 1 microgram to 10 mg of a therapeutic. Nebulizers are devices that turn medication into a fine mist inhalable by a subject through a face mask that covers the mouth and nose. Nebulizers provide small droplets and high mass output for delivery to upper and lower respiratory airways. Typically, nebulizers create droplets down to about 1 micron in diameter.

Dry-powder inhalers (DPI) can be used to deliver the compounds of the invention, either alone or in combination with a pharmaceutically acceptable carrier. DPIs deliver active ingredient to airways and lungs while the subject inhales through the device. DPIs typically do not contain propellants or other ingredients, only medication, but may optionally include other components. DPIs are typically breath-activated, but may involve air or gas pressure to assist delivery.

For rectal administration, pharmaceutical compositions can be included as a suppository with a suitable base comprising, for example, cocoa butter or a salicylate. For vaginal administration, pharmaceutical compositions can be included as pessaries, tampons, creams, gels, pastes, foams or spray formulations containing in addition to the active ingredient (e.g., CSA) a carrier, examples of appropriate carriers which are known in the art.

Pharmaceutical formulations and delivery systems appropriate for the compositions and methods of the invention are known in the art (see, e.g., Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index (1996) 12^{th} ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, Md.; and Poznansky et al., Drug Delivery Systems (1980), R. L. Juliano, ed., Oxford, N.Y., pp. 253−315).

Compounds of the invention (e.g., CSAs), including pharmaceutical formulations can be packaged in unit dosage forms for ease of administration and uniformity of dosage. A “unit dosage form” as used herein refers to a physically discrete unit suited as unitary dosages for the subject to be treated; each unit containing a predetermined quantity of compound optionally in association with a pharmaceutical carrier (excipient, diluent, vehicle or filling agent) which, when administered in one or more doses, is calculated to produce a desired effect (e.g., prophylactic or therapeutic effect or benefit). Unit dosage forms can contain a daily dose or unit, daily sub-dose, or an appropriate fraction thereof, of an administered compound (e.g., CSA). Unit dosage forms also include, for example, capsules, troches, cachets, lozenges, tablets, ampules and vials, which may include a composition in a freeze-dried or lyophilized state; a sterile liquid carrier, for example, can be added prior to administration or delivery in vivo. Unit dosage forms additionally include, for example, ampules and vials with liquid compositions disposed therein. Unit dosage forms further include compounds for transdermal administration, such as “patches” that contact with the epidermis of the subject for an extended or brief period of time. The individual unit dosage forms can be included in multi-dose kits or containers. Pharmaceutical formulations can be packaged in single or multiple unit dosage forms for ease of administration and uniformity of dosage.

Compounds of the invention (e.g., CSAs) can be administered in accordance with the methods at any frequency as a single bolus or multiple dose e.g., one, two, three, four, five, or more times hourly, daily, weekly, monthly or annually or between about 1 to 10 days, weeks, months, or for as long as appropriate. Exemplary frequencies are typically from 1−7 times, 1−5 times, 1−3 times, 2-times or once, daily, weekly or monthly. Timing of contact, administration ex vivo or in vivo delivery can be dictated by the infection, pathogenesis (e.g., illness), symptom, pathology or adverse side effect to be treated. For example, an amount can be administered to the subject substantially contemporaneously with, or within about 1−60 minutes or hours of the onset of a symptom or adverse side effect of HIV infection, pathogenesis (e.g., illness) or vaccination.

Doses may vary depending upon whether the treatment is therapeutic or prophylactic, the onset, progression, severity, frequency, duration, probability of or susceptibility of the symptom, the type of virus infection or pathogenesis (e.g., illness) to which treatment is directed, clinical endpoint desired, previous, simultaneous or subsequent treatments, general health, age, gender or race of the subject, bioavailability, potential adverse systemic, regional or local side effects, the presence of other disorders or diseases in the subject, and other factors that will be appreciated by the skilled artisan (e.g., medical or familial history). Dose amount, frequency or duration may be increased or reduced, as indicated by the clinical outcome desired, status of the infection, symptom or pathology, any adverse side effects of the treatment or therapy. The skilled artisan will appreciate the factors that may influence the dosage, frequency and timing required to provide an amount sufficient or effective for providing a prophylactic or therapeutic effect or benefit.

For therapeutic treatment, a compound of the invention (e.g., CSA) will be administered as soon as practical, typically within 0−72 hours or days after a subject is exposed to, contacted or infected with HIV (e.g., diagnosed as HIV+), or within 0−72 hours or days after development of one or more symptoms or pathologies associated with HIV infection or pathogenesis (e.g., illness such as fever, fatigue, swollen lymph nodes, reduced CD4+ Tcell numbers, opportunistic infections).

For prophylactic treatment, a compound of the invention can be administered immediately or within 0−72 after suspected contact with, or 0−4 weeks, e.g., 1−3 days or weeks, prior to anticipated or possible exposure to or contact with HIV. For prophylactic treatment in connection with immunization/vaccination of a subject, a compound can be administered prior to, concurrently with or following immunization/vaccination of the subject.

Doses can be based upon current existing treatment protocols, empirically determined, determined using animal disease models or optionally in human clinical studies. For example, initial study doses can be based upon animal studies, such as primates, and the amount of compound administered to achieve a prophylactic or therapeutic effect or benefit. The dose can be adjusted according to the mass of a subject, and will generally be in a range from about 0.1-1 ug/kg, 1-10 ug/kg, 10-25 ug/kg, 25-50 ug/kg, 50−100 ug/kg, 100-500 ug/kg, 500-1,000 ug/kg, 1−5 mg/kg, 5-10 mg/kg, 10-20 mg/kg, 20−50 mg/kg, 50-100 mg/kg, 100-250 mg/kg, 250−500 mg/kg, or more, of subject body weight, two, three, four, or more times per hour, day, week, month or annually. Of course, doses can be more or less, as appropriate, for example, 0.00001 mg/kg of subject body weight to about 10,000.0 mg/kg of subject body weight, about 0.001 mg/kg, to about 100 mg/kg, about 0.01 mg/kg, to about 10 mg/kg, or about 0.1 mg/kg, to about 1 mg/kg of subject body weight over a given time period, e.g., 1, 2, 3, 4, 5 or more hours, days, weeks, months, years. A subject may be administered in single bolus or in divided/metered doses, which can be adjusted to be more or less according to the various consideration set forth herein and known in the art.

Dose amount, frequency or duration may be increased or reduced, as indicated by the status of the HIV infection or pathogenesis (e.g., illness), associated symptom or pathology, or any adverse side effect(s) of HIV, or an HIV treatment or anti-HIV therapy. For example, once control or a particular endpoint is achieved, for example, reducing, decreasing, inhibiting, ameliorating or preventing onset, severity, duration, progression, frequency or probability of one or more symptoms associated with an HIV infection or pathogenesis (e.g., illness) of one or more symptoms or pathologies associated with or caused by HIV infection or pathogenesis, dose amount, frequency or duration can be reduced.

The invention provides kits including compounds of the invention (e.g., CSA), combination compositions and pharmaceutical compositions/formulations thereof, packaged into a suitable packaging material. In one embodiment, a kit includes packaging material, a cationic steroid antimicrobial (CSA) and instructions. In various aspects, the instructions are for administering the CSA to: provide a subject with protection against an HIV infection or pathogenesis (e.g., illness); treat a subject for HIV infection or pathogenesis (e.g., illness); decrease susceptibility of a subject to an HIV infection or pathogenesis (e.g., illness); or decrease or prevent an adverse side effect caused by or associated with HIV or an HIV treatment.

The term “packaging material” refers to a physical structure housing one or more components of the kit. The packaging material can maintain the components sterilely, and can be made of material commonly used for such purposes (e.g., paper, corrugated fiber, glass, plastic, foil, ampules, vials, tubes, etc.). A kit can contain a plurality of components, e.g., two or more compounds of the invention alone or in combination with an anti-HIV agent or treatment (e.g., an anti-viral, an HIV protein or an antibody that binds to an HIV protein) or drug, optionally sterile.

A kit optionally includes a label or insert including a description of the components (type, amounts, doses, etc.), instructions for use in vitro, in vivo, or ex vivo, and any other components therein. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a disk (e.g., floppy diskette, hard disk, ZIP disk), optical disk such as CD- or DVD-ROM/RAM, DVD, MP3, magnetic tape, or an electrical storage media such as RAM and ROM or hybrids of these such as magnetic/optical storage media, FLASH media or memory type cards.

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer, lot numbers, manufacturer location and date, expiration dates.

Labels or inserts can include information on a condition, disorder or disease (e.g., virus pathogenesis or infection) for which a kit component may be used. Labels or inserts can include instructions for a clinician or subject for using one or more of the kit components in a method, treatment protocol or therapeutic/prophylactic regimen, including the methods of the invention. Instructions can include amounts of compound, frequency or duration of administration, and instructions for practicing any of the methods, treatment protocols or prophylactic or therapeutic regimes described herein. Exemplary instructions include, instructions for treating HIV infection or pathogenesis (e.g., illness). Kits of the invention therefore can additionally include labels or instructions for practicing any of the methods of the invention described herein including treatment, screening or other methods. Thus, for example, a kit can include a compound of the invention (e.g., CSA) that has one or more anti-HIV activities as set forth herein, together with instructions for administering the compound in a prophylactic or therapeutic treatment method of the invention, for example to a subject in need of such treatment. Exemplary instructions include administering the CSA to: provide a subject with protection against an HIV infection or pathogenesis; treat a subject for HIV infection or pathogenesis; decrease susceptibility of a subject to an HIV infection or pathogenesis; decrease, inhibit, ameliorate or prevent onset, severity, duration, progression, frequency or probability of one or more symptoms associated with HIV infection or pathogenesis; or decrease or prevent an adverse side effect caused by or associated with HIV or an HIV treatment.

Labels or inserts can include information on any effect or benefit a kit component may provide, such as a prophylactic or therapeutic effect or benefit. For example, a label or insert could provide a description of one or more symptoms which can be improved, i.e., reducing, decreasing, inhibiting, ameliorating or preventing onset, severity, duration, progression, frequency or probability of one or more symptoms or pathologies associated with an HIV infection or pathogenesis, or one or more adverse side effects associated with HIV or an HIV treatment. HIV symptoms and pathologies are as set forth herein or known in the art (e.g., fever, fatigue, headache, sore throat, swollen lymph nodes, weight loss, diarrhea, rash, boils, warts, thrush, shingles, chronic or acute pelvic inflammatory disease (PID), dry cough, shortness of breath, bruising, bleeding, numbness or paralysis, muscle weakness, an opportunistic disorder, nerve damage, encephalopathy, dementia, death, etc.). Adverse side effects associated with HIV and anti-HIV treatments are set forth herein or known in the art.

Labels or inserts can include information on potential adverse side effects of treatment. Labels or inserts can further include warnings to the clinician or subject regarding situations or conditions where a subject should stop or reduce use of a particular kit component. Adverse side effects could also occur when the subject has, will be or is currently taking one or more other medications that may be incompatible with a compound of the invention, or the subject has, will be or is currently undergoing another treatment protocol or therapeutic regimen which would be incompatible with the compound and, therefore, labels or inserts could include information regarding such side effects or incompatibilities.

Invention kits can additionally include a buffering agent, or a preservative or a stabilizing agent in a pharmaceutical formulation containing a compound of the invention. Each component of the kit can be enclosed within an individual container and all of the various containers can be within a single package. Invention kits can be designed for cold storage.

Invention kits can include components, such as devices for practicing a method of the invention or administering a compound of the invention (e.g., CSA) to a subject, ex vivo or in vivo. The device can be a delivery device, such as a syringe, a compressible (e.g., squeezable) tube or dermal patch for mucosal, skin/dermis or corneal delivery, or an aerosol delivery device for administration to lungs or airways.

Compounds useful in accordance with the invention, are described herein, both generically and with particularity, and in U.S. Pat. Nos. 6,350,738; 6,486,148; and 6,767,904, which are incorporated herein by reference. Compounds include steroid derivatives, such as cationic steroid antimicrobials (CSA) that exhibit one or more anti-HIV activities or functions. The skilled artisan will recognize the compounds within the generic formula set forth herein. Additional compounds of the invention having one or more anti-HIV activities or functions are described and can be characterized using the assays set forth herein and in the art.

Compounds of formula I, also referred to as cationic steroid antimicrobials (CSA), comprise: 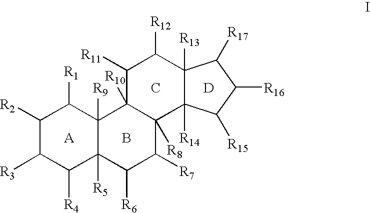
wherein:
fused rings A, B, C, and D are independently saturated or fully or partially unsaturated; and
each of R₁ through R₄, R₆, R₇, R₁₁, R₁₂, R₁₅, R₁₆, and R₁₇ is independently selected from the group consisting of hydrogen, hydroxyl, a substituted or unsubstituted (C1-C10) alkyl, (C1-C10) hydroxyalkyl, (C1-C10) alkyloxy-(C1-C10) alkyl, (C1-C10) alkylcarboxy-(C1-C10) alkyl, (C1-C10) alkylamino-(C1-C10) alkyl, (C1-C10) alkylamino-(C1-C10) alkylamino, (C1-C10) alkylamino-(C1-C10) alkylamino-(C1-C10) alkylamino, a substituted or unsubstituted (C1-C10) aminoalkyl, a substituted or unsubstituted aryl, a substituted or unsubstituted arylamino-(C1-C10) alkyl, (C1-C10) haloalkyl, C2-C6 alkenyl, C2-C6 alkynyl, oxo, a linking group attached to a second steroid, a substituted or unsubstituted (C1-C10) aminoalkyloxy, a substituted or unsubstituted (C1-C10) aminoalkyloxy-(C1-C10) alkyl, a substituted or unsubstituted (C1-C10) aminoalkylcarboxy, a substituted or unsubstituted (C1-C10) aminoalkylaminocarbonyl, a substituted or unsubstituted (C1-C10) aminoalkylcarboxamido, H₂N—HC(Q5)-C(O)—O—, H₂N—HC(Q5)-C(O)—N(H)—, (C1-C10) azidoalkyloxy, (C1-C10) cyanoalkyloxy, P.G.-HN—HC(Q5)-C(O)—O—, (C1-C10) guanidinoalkyloxy, (C1-C10) quaternaryammoniumalkylcarboxy, and (C1-C10) guanidinoalkyl carboxy, where Q5 is a side chain of any amino acid (including the side chain of glycine, i.e., H), P.G. is an amino protecting group, and
R₅, R₈, R₉, R₁₀, R₁₃, and R₁₄ is each independently: deleted when one of fused rings A, B, C, or D is unsaturated so as to complete the valency of the carbon atom at that site, or
selected from the group consisting of hydrogen, hydroxyl, a substituted or unsubstituted (C1-C10) alkyl, (C1-C10) hydroxyalkyl, (C1-C10) alkyloxy-(C1-C10) alkyl, a substituted or unsubstituted (C1-C10) aminoalkyl, a substituted or unsubstituted aryl, C1-C10 haloalkyl, C2-C6 alkenyl, C2-C6 alkynyl, oxo, a linking group attached to a second steroid, a substituted or unsubstituted (C1-C10) aminoalkyloxy, a substituted or unsubstituted (C1-C10) aminoalkylcarboxy, a substituted or unsubstituted (C1-C10) aminoalkylaminocarbonyl, H₂N—HC(Q5)-C(O)—O—, H2N—HC(Q5)-C(O)—N(H)—, (C1-C10) azidoalkyloxy, (C1-C10) cyanoalkyloxy, P.G.-HN—HC(Q5)-C(O) —O—, (C1-C10) guanidinoalkyloxy, and (C1-C10) guanidinoalkylcarboxy, where Q5 is a side chain of any amino acid, P.G. is an amino protecting group, and
provided that at least two of R₁ through R₁₄ are independently selected from the group consisting of a substituted or unsubstituted (C1-C10) aminoalkyloxy, (C1-C10) alkylcarboxy-(C1-C10) alkyl, (C1-C10) alkylamino-(C1-C10) alkylamino, (C1-C10) alkylamino-(C1-C10) alkylamino-(C1-C10) alkylamino, a substituted or unsubstituted (C1-C10) aminoalkylcarboxy, a substituted or unsubstituted arylamino-(C1-C10) alkyl, a substituted or unsubstituted (C1-C10) aminoalkyloxy-(C1-C10) alkyl, a substituted or unsubstituted (C1-C10) aminoalkylaminocarbonyl, (C1-C10) quaternaryammonium alkylcarboxy, H2N—HC(Q5)-C(O)—O—, H₂N—HC(Q5)-C(O)—N(H)—, (C1-C10) azidoalkyloxy, (C1-C10) cyanoalkyloxy, P.G.-HN—HC(Q5)-C(O)—O—, (C1-C10) guanidinoalkyloxy, and (C1-C10) guanidinoalkylcarboxy; or a pharmaceutically acceptable salt thereof.

A “ring” as used herein can be heterocyclic or carbocyclic. The term “saturated” used herein refers to the fused ring of formula I having each atom in the fused ring either hydrogenated or substituted such that the valency of each atom is filled. The term “unsaturated” used herein refers to the fused ring of formula I where the valency of each atom of the fused ring may not be filled with hydrogen or other substituents. For example, adjacent carbon atoms in the fused ring can be doubly bound to each other. Unsaturation can also include deleting at least one of the following pairs and completing the valency of the ring carbon atoms at these deleted positions with a double bond; such as R₅ and R₉; R₈ and R₁₀; and R₁₃ and R₁₄.

The term “unsubstituted” used herein refers to a moiety having each atom hydrogenated such that the valency of each atom is filled.

The term “halo” used herein refers to a halogen atom such as fluorine, chlorine, bromine, or iodine.

Examples of amino acid side chains include but are not limited to H (glycine), methyl (alanine), —CH₂—(C═O)—NH₂ (asparagine), —CH₂—SH (cysteine), and —CH(OH)CH₃ (threonine).

An alkyl group is a branched or unbranched hydrocarbon that may be substituted or unsubstituted. Examples of branched alkyl groups include isopropyl, sec-butyl, isobutyl, tert-butyl, sec-pentyl, isopentyl, tert-pentyl, isohexyl. Substituted alkyl groups may have one, two, three or more substituents, which may be the same or different, each replacing a hydrogen atom. Substituents are halogen (e.g., F, Cl, Br, and I), hydroxyl, protected hydroxyl, amino, protected amino, carboxy, protected carboxy, cyano, methylsulfonylamino, alkoxy, acyloxy, nitro, and lower haloalkyl.

The term “substituted” used herein refers to moieties having one, two, three or more substituents, which may be the same or different, each replacing a hydrogen atom. Examples of substituents include but are not limited to halogen (e.g., F, Cl, Br, and I), hydroxyl, protected hydroxyl, amino, protected amino, carboxy, protected carboxy, cyano, methylsulfonylamino, alkoxy, alkyl, aryl, aralkyl, acyloxy, nitro, and lower haloalkyl.

An aryl group is a C6-20 aromatic ring, wherein the ring is made of carbon atoms (e.g., C6-C14, C6-10 aryl groups). Examples of haloalkyl include fluoromethyl, dichloromethyl, trifluoromethyl, 1,1-difluoroethyl, and 2,2-dibromoethyl.

An aralkyl group is a group containing 6-20 carbon atoms that has at least one aryl ring and at least one alkyl or alkylene chain connected to that ring. An example of an aralkyl group is a benzyl group.

A linking group is any divalent moiety used to link a compound of formula to another steroid, e.g., a second compound of formula I. An example of a linking group is (C1-C10) alkyloxy-(C1-C10) alkyl.

Amino-protecting groups are known to those skilled in the art. In general, the species of protecting group is not critical, provided that it is stable to the conditions of any subsequent reaction(s) on other positions of the compound and can be removed at the appropriate point without adversely affecting the remainder of the molecule. In addition, a protecting group may be substituted for another after substantive synthestic transformations are complete. Clearly, where a compound differs from a compound disclosed herein only in that one or more protecting groups of the disclosed compound has been substituted with a different protecting group, that compound is within the invention. Further examples and conditions are found in T. W. Greene, Protective Groups in Organic Chemistry, (1st ed., 1981, 2nd ed., 1991).

The invention also includes compounds comprising a ring system of at least 4 fused rings, where each of the rings has from 5−7 atoms. The ring system has two faces, and contains 3 chains attached to the same face. Each of the chains contains a nitrogen-containing group that is separated from the ring system by at least one atom; the nitrogen-containing group is an amino group, e.g., a primary amino group, or a guanidino group. The compound can also contain a hydrophobic group, such as a substituted (C3-10) aminoalkyl group, a (C1-C10) alkyloxy (C3-10) alkyl group, or a (C1-C10) alkylamino (C3-10)alkyl group, attached to the steroid backbone.

For example, the compound may have the formula V, where each of the three chains containing nitrogen-containing groups is independently selected from R₁ through R₄, R₆, R₇, R₁₁, R₁₂, R₁₅, R₁₆, R₁₇, and R₁₈, defined below. 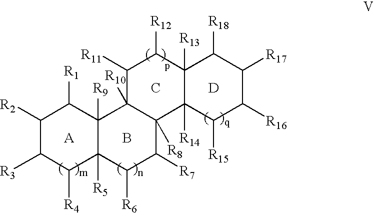
where:
each of fused rings A, B, C, and D is independently saturated, or is fully or partially unsaturated, provided that at least two of A, B, C, and D are saturated, wherein rings A, B, C, and D form a ring system;
each of m, n, p, and q is independently 0 or 1;
each of R₁ through R₄, R₆, R₇, R₁₁, R₁₂, R₁₅, R₁₆, R₁₇, and R₁₈ is independently selected from the group consisting of hydrogen, hydroxyl, a substituted or unsubstituted (C1-C10) alkyl, (C1-C10) hydroxyalkyl, (C1-C10) alkyloxy-(C1-C10) alkyl, (C1-C10)alkylcarboxy-(C1-C10 alkyl, (C1-C10) alkylamino-(C1-C10) alkyl, (C1-C10) alkylamino-(C1-C10) alkylamino, (C1-C10 alkylamino-(C1-C10) alkylamino-(C1-C10) alkylamino, a substituted or unsubstituted (C1-C10) aminoalkyl, a substituted or unsubstituted aryl, a substituted or unsubstituted aryl amino-(C1-C10) alkyl, (C1-C10) haloalkyl, C2-C6 alkenyl, C2-C6 alkynyl, oxo, a linking group attached to a second steroid, a substituted or unsubstituted (C1-C10) aminoalkyloxy, a substituted or unsubstituted (C1-C10) aminoalkyloxy-(C1-C10) alkyl, a substituted or unsubstituted (C1-C10) aminoalkylcarboxy, a substituted or unsubstituted (C1-C10)
aminoalkylaminocarbonyl, a substituted or unsubstituted (C1-C10) aminoalkylcarboxamido, H₂N—HC(Q5)-C(O)—O—, H₂N—HC(Q5)-C(O)—N(H)—, (C1-C10) azidoalkyloxy, (C1-C10) cyanoalkyloxy, P.G.-HN—HC(Q5)-C(O)—O—, (C1-C10) guanidinoalkyl oxy, (C1-C10) quaternaryammoniumalkylcarboxy, and (C1-C10) guanidinoalkyl carboxy, where Q5 is a side chain of any amino acid (including a side chain of glycine, i.e., H). P.G. is an amino protecting group: and
each of R₅, R₈, R₉, R₁₀, R₁₃, and R₁₄ is independently: deleted when one of fused rings A, B, C, or D is unsaturated so as to complete the valency of the carbon atom at that site, or selected from the group consisting of hydrogen, hydroxyl, a substituted or unsubstituted (C1-C10) alkyl, (C1-C10) hydroxyalkyl, (C1-C10) alkyloxy-(C1-C10) alkyl, a substituted or unsubstituted (C1-C10) aminoalkyl, a substituted or unsubstituted aryl, C1-C10 haloalkyl, C2-C6 alkenyl, C2-C6 alkynyl, oxo, a linking group attached to a second steroid, a substituted or unsubstituted (C1-C10) aminoalkyloxy, a substituted or unsubstituted (C1-C10) aminoalkylcarboxy, a substituted or unsubstituted (C1-C10) aminoalkylaminocarbonyl, H2N—HC(Q5)-C(O)—O—, H₂N—HC(Q5)-C(O)—N(H)—, (C1-C10) azidoalkyloxy, (C1-C10) cyanoalkyloxy, P.G.-HN—HC(Q5)-C(O) —O—, (C1-C10) guanidinoalkyloxy, and (C1-C10) guanidinoalkylcarboxy, where Q5 is a side chain of any amino acid, P.G. is an amino protecting group,
provided that at least three of R₁ through R₄, R₆, R₇, R₁₁, R₁₂, R₁₅, R₁₆, R₁₇, and R₁₈ are disposed on the same face of the ring system and are independently selected from the group consisting of a substituted or unsubstituted (C1-C10) aminoalkyl, a substituted or unsubstituted (C1-C10) aminoalkyloxy, (C1-C10) alkylcarboxy-(C1-C10) alkyl, (C1-C10) alkylamino-(C1-C10) alkyl amino, (C1-C10) alkylamino-(C1-C10) alkylamino-(C1-C10) alkylamino, a substituted or unsubstituted (C1-C10) aminoalkylcarboxy, a substituted or unsubstituted arylamino-(C1-C10) alkyl, a substituted or unsubstituted (C1-C10) aminoalkyloxy-(C1-C10) aminoalkylaminocarbonyl, a substituted or unsubstituted (C1-C10) aminoalkylaminocarbonyl, a substituted or unsubstituted (C1-C5) aminoalkylcarboxamido, a (C1-C10) quaternaryammoniumalkylcarboxy, H2N—HC(Q5)-C(O)—O—, H₂N—HC(Q5)-C(O)—N(H)—, (C1-C10) azidoalkyloxy, (C1-C10) cyanoalkyloxy, P.G.-HN—HC(Q5)-C(O)—O—, (C1-C10) guanidinoalkyloxy, and a (C1-C10) guanidinoalkylcarboxy; or a pharmaceutically acceptable salt thereof. In various aspects, at least two, or at least, three, of m, n, p, and q are 1.

Compounds set forth herein preserve certain stereochemical and electronic characteristics found in steroids. The term “same configuration” as used herein refers to substituents on the fused steroid having the same stereochemical orientation. For example substituents R₃, R₇ and R₁₂ are all β-substituted or α-substituted.

Compounds of the invention include but are not limited to compounds having amine or guanidine groups covalently attached to a steroid backbone or scaffold at any carbon position, e.g., cholic acid. In various embodiments, a group is covalently attached at any one, or more, of positions C3, C7 and C12 of the steroid backbone or scaffold. In additional embodiments, a group is absent from any one, or more, of positions C3, C7 and C12 of the steroid backbone or scaffold.

Compounds of the invention that include such groups can include a tether, the tether having variable chain length or size. As used herein, the terms “tether” or “tethered,” when used in reference to a compound of the invention, refers to the chain of atoms between the steroid backbone or scaffold and a terminal amino or guanidine group. In various embodiments, a tether is covalently attached at any one, or more, of positions C3, C7 and C12. In additional embodiments, a tether is lacking at any one, or more, of positions C3, C7 and C12. A tether length may include the heteroatom (O or N) covalently attached to the steroid backbone.

Other ring systems can also be used, e.g., 5-member fused rings. Compounds with backbones having a combination of 5- and 6-membered rings are also included in the invention. Amine or guanidine groups can be separated from the backbone by at least one, two, three, four or more atoms. The backbone can be used to orient the amine or guanidine groups on one face, or plane, of the steroid. For example, a scheme showing a compound having primary amino groups on one face, or plane, of a backbone is shown below:

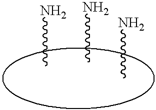

Methods of synthesizing compounds of formula I are provided, wherein for example, at least two of R₁ through R₁₄ are independently selected from the group consisting of a substituted or unsubstituted (C1-C10) aminoalkyloxy. In one embodiment, a method includes the step of contacting a compound of formula IV, 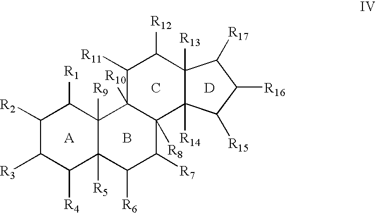
where at least two of R₁ through R₁₄ are hydroxyl, and the remaining moieties on the fused rings A, B, C, and D are defined for formula I, with an electrophile to produce an alkyl ether compound of formula IV, wherein at least two of R₁ through R₁₄ are (C1-C10)alkyloxy. The alkyl ether compounds are converted into an amino precursor compound wherein at least two of R₁ through R₁₄ are independently selected from the group consisting of (C1-C10) azidoalkyloxy and (C1-C10) cyanoalkyloxy and the amino precursor compound is reduced to form a compound of formula I.

The electrophiles used in a method include but are not limited to 2-(2-bromoethyl)-1,3-dioxolane, 2-iodoacetamide, 2-chloroacetamide, N-(2-bromoethyl)phthalimide, N-(3-bromopropyl)phthalimide, and allybromide. An exemplary electrophile is alkylbromide.

The invention also includes methods of producing a compound of formula I where at least two of R₁ through R₁₄ are (C1-C10) guanidoalkyloxy. In one embodiment, a method includes contacting a compound of formula IV, where at least two of R₁ through R₁₄ are hydroxyl, with an electrophile to produce an alkyl ether compound of formula IV, where at least two of R₁ through R₁₄ are (C1-C10)alkyloxy. The allyl ether compound is converted into an amino precursor compound where at least two of R₁ through R₁₄ are independently selected from the group consisting of (C1-C10) azidoalkyloxy and (C1-C10) cyanoalkyloxy. The amino precursor compound is reduced to produce an aminoalkyl ether compound wherein at least two of R₁ through R₁₄ are (C1-C10) aminoalkyloxy. The aminoalkyl ether compound is contacted with a guanidino producing electrophile to form a compound of formula I.

The term “guanidino producing electrophile” used herein refers to an electrophile used to produce a guanidino compound of formula I. An example of an guanidino producing electrophile is HSO₃—C(NH)—NH₂.

The invention also includes methods of producing a compound of formula I where at least two of R₁ through R₁₄ are H2N—HC(Q5)-C(O)—O— and Q5 is the side chain of any amino acid. In one embodiment, a method includes the step of contacting a compound of formula IV, where at least two of R₁ through R₁₄ are hydroxyl, with a protected amino acid to produce a protected amino acid compound of formula IV where at least two of at least two of R₁ through R₁₄ are P.G.-HN—HC(Q5)-C(O)—O— and Q5 is the side chain of any amino acid and P.G. is an amino protecting group. The protecting group of the protected amino acid compound is removed to form a compound of formula I.

Exemplary non-limiting synthesis schemes for preparing compounds of the invention include the following:

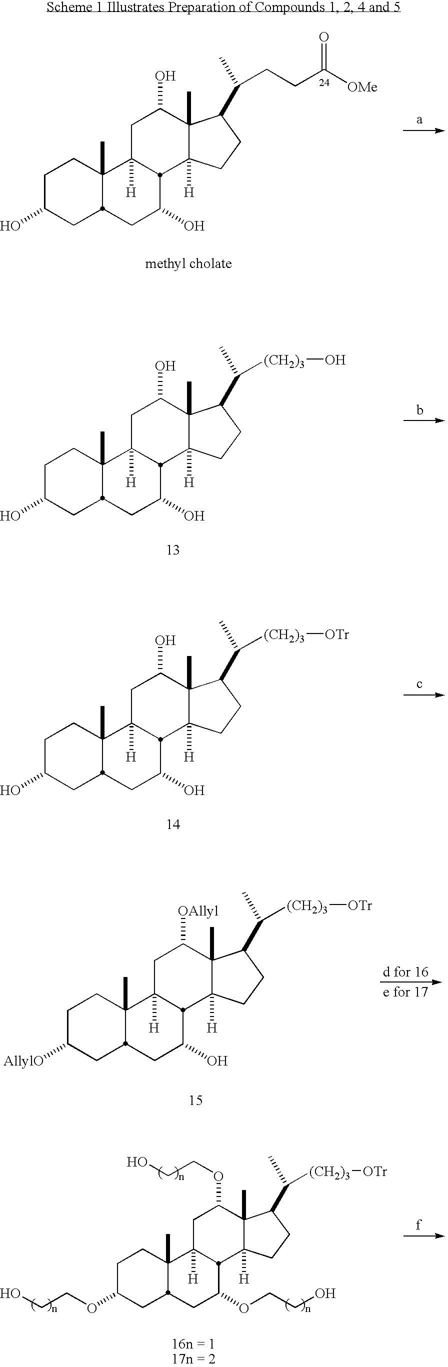

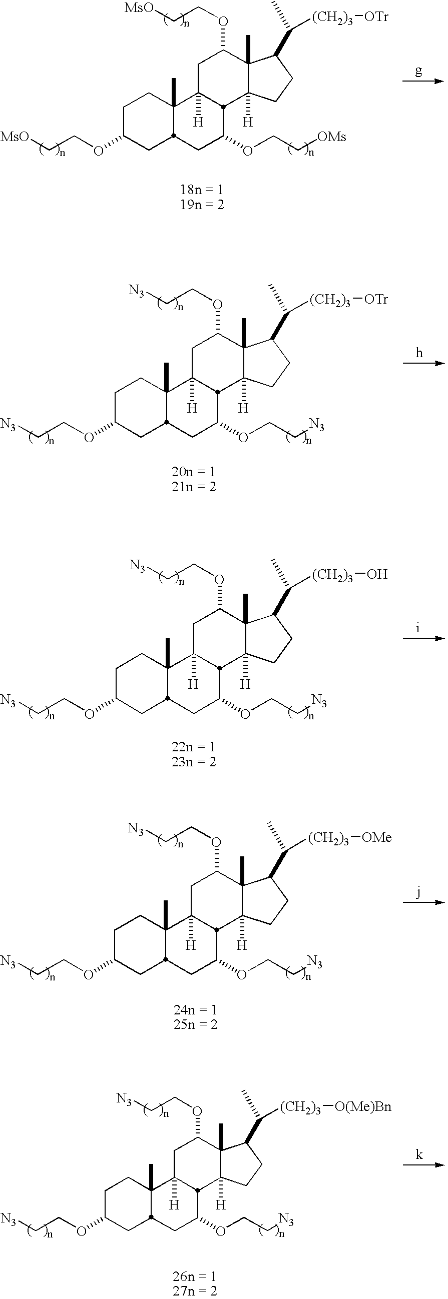

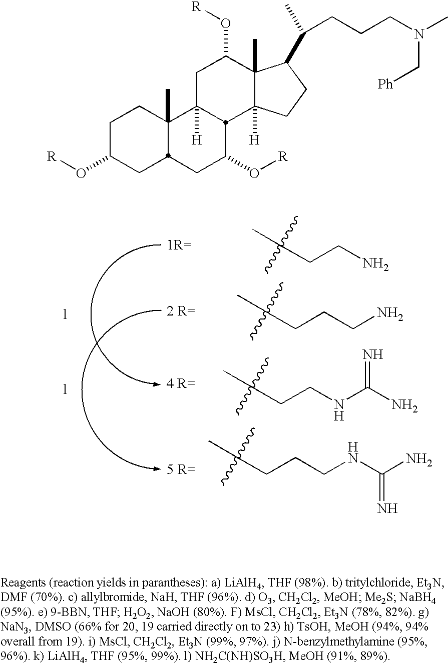

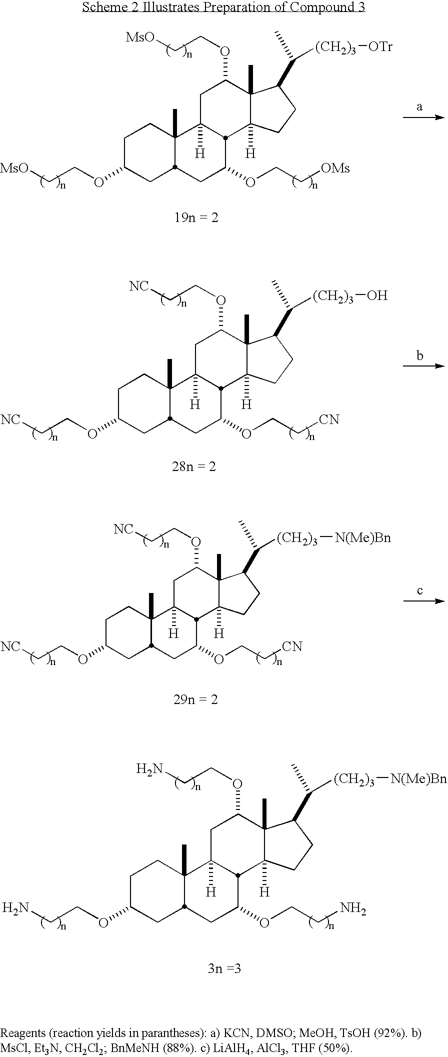

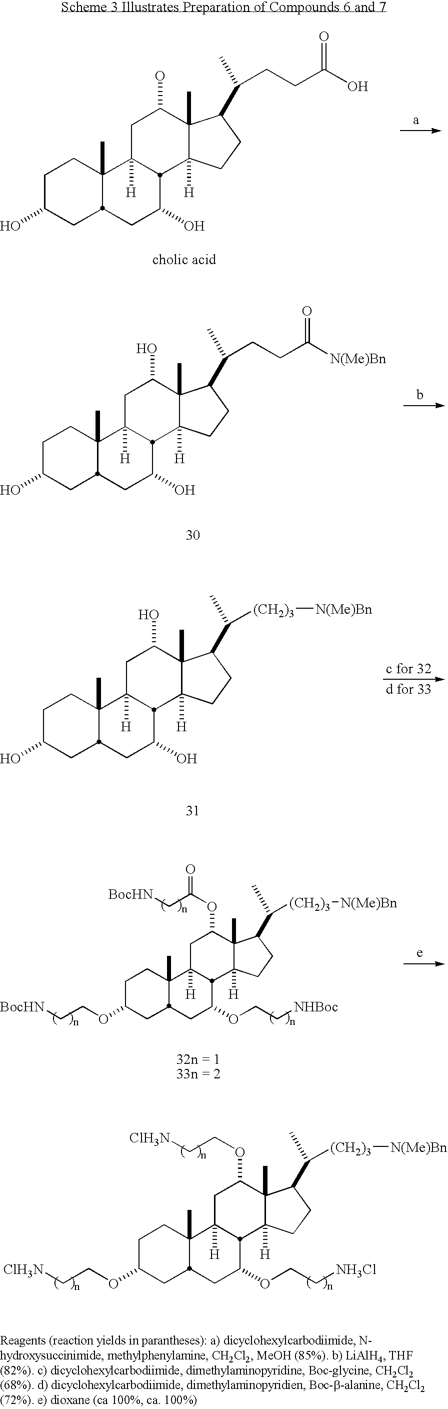

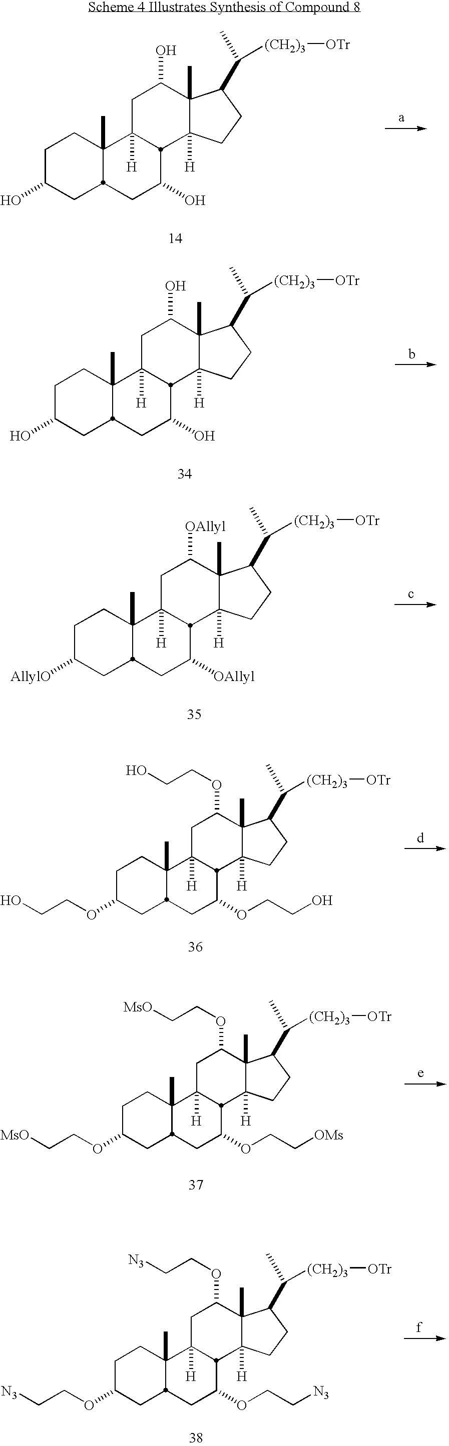

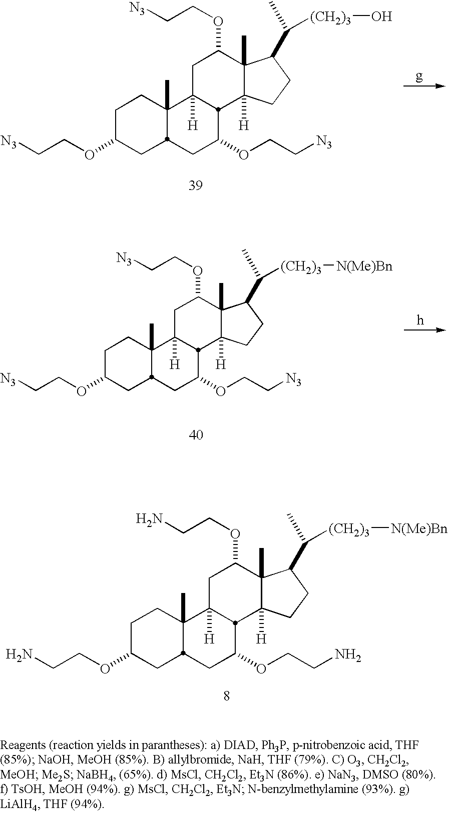

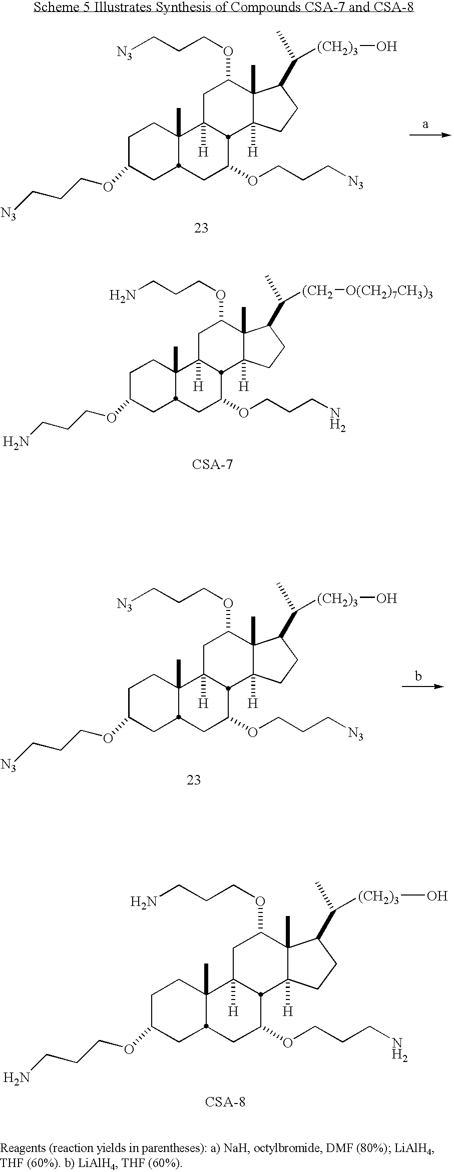

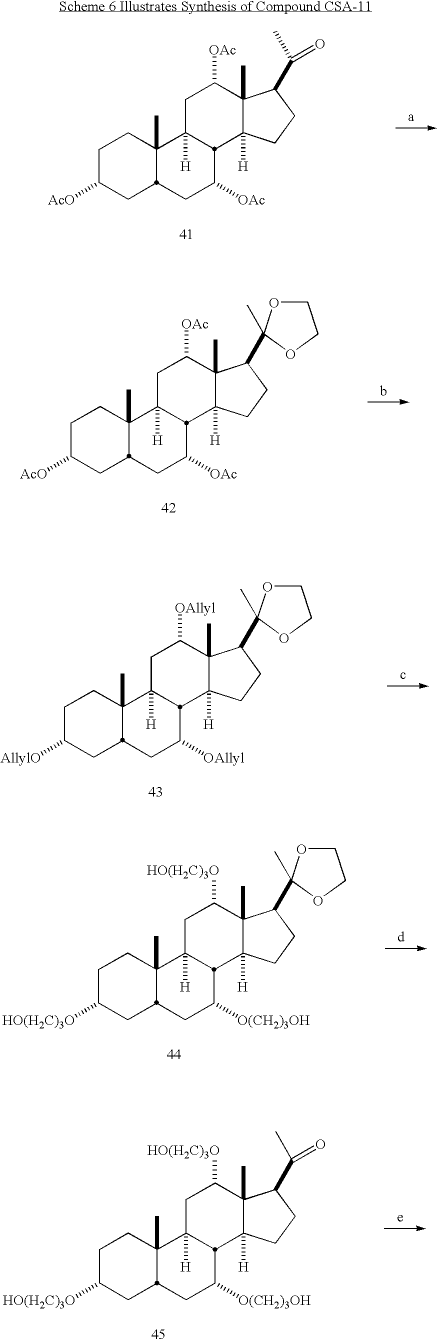

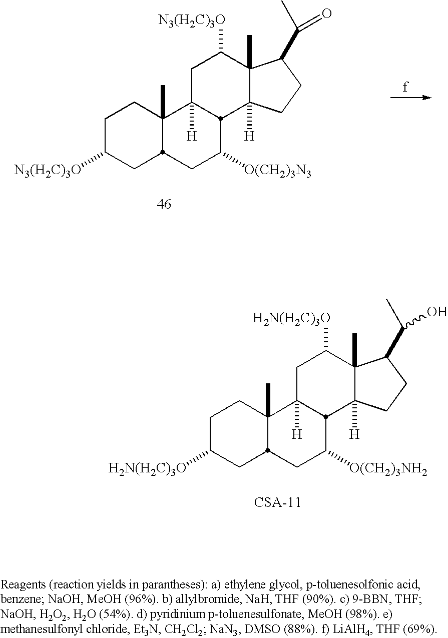

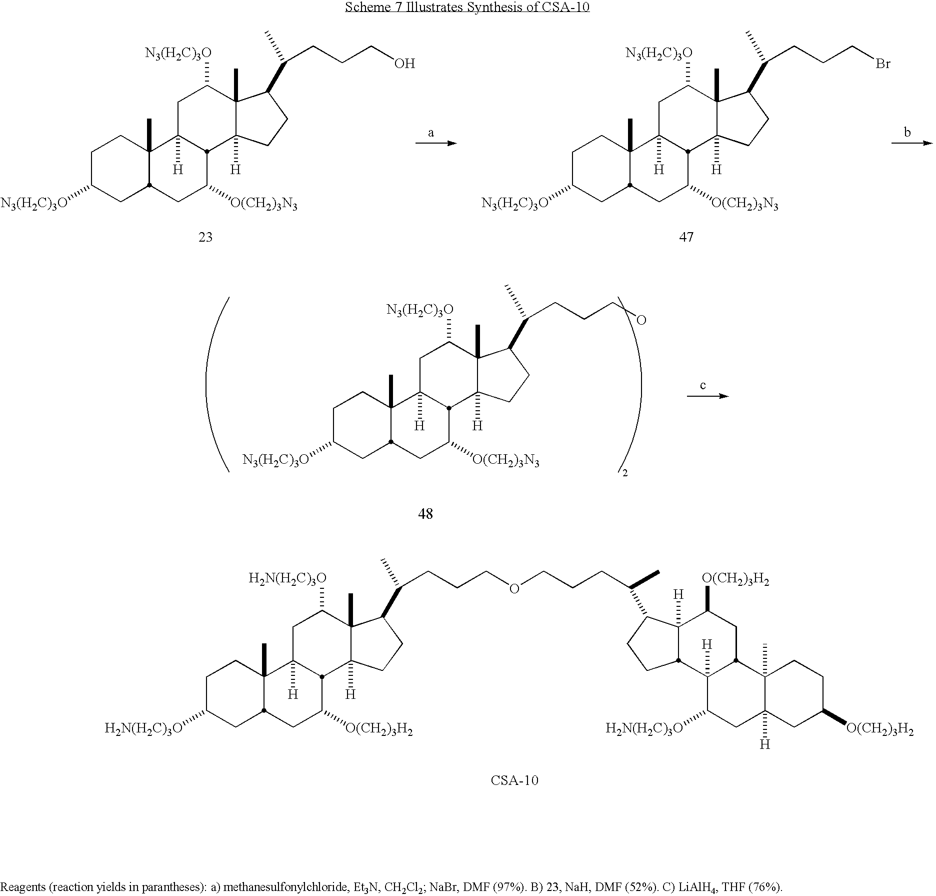

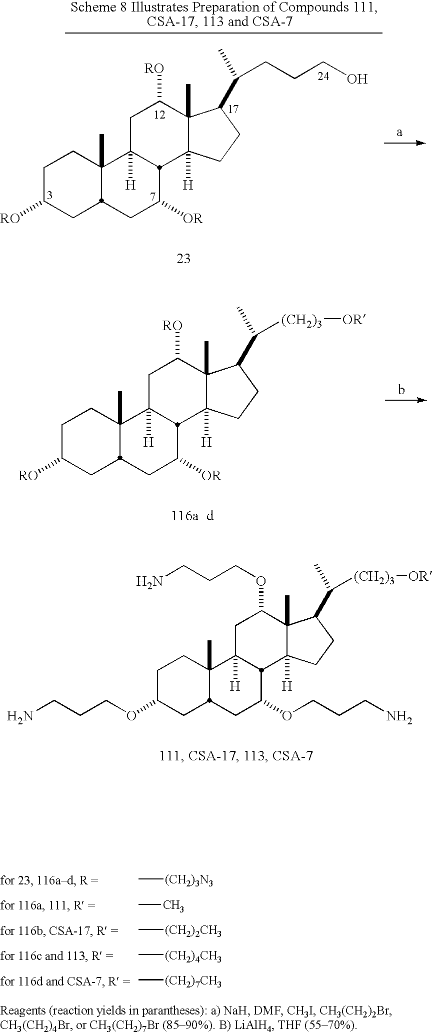

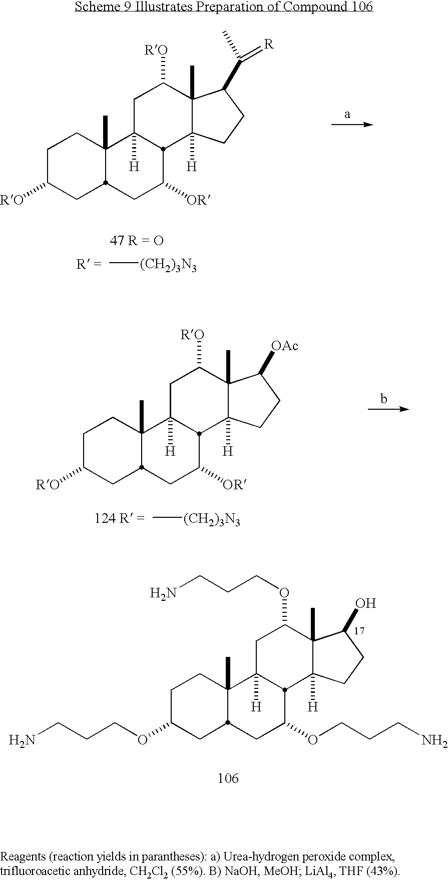

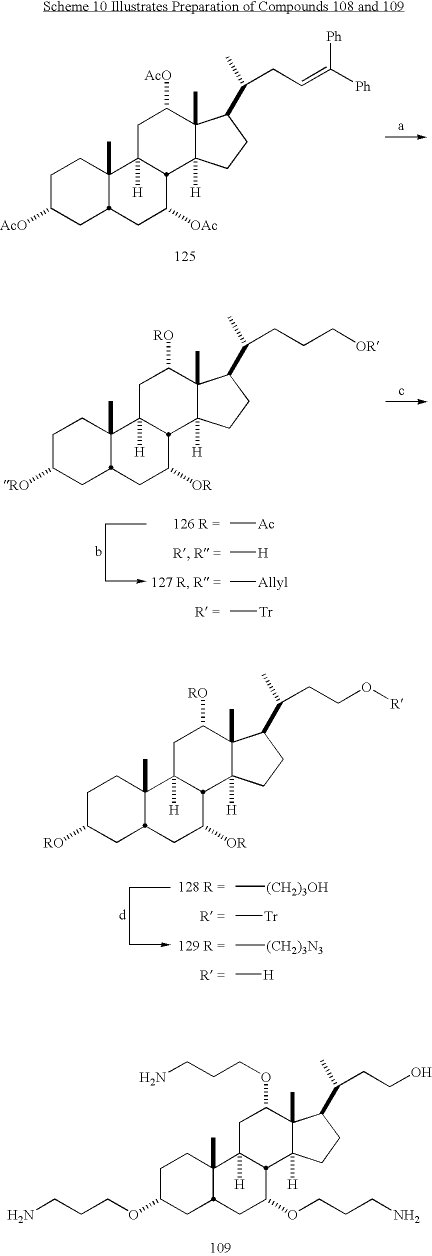

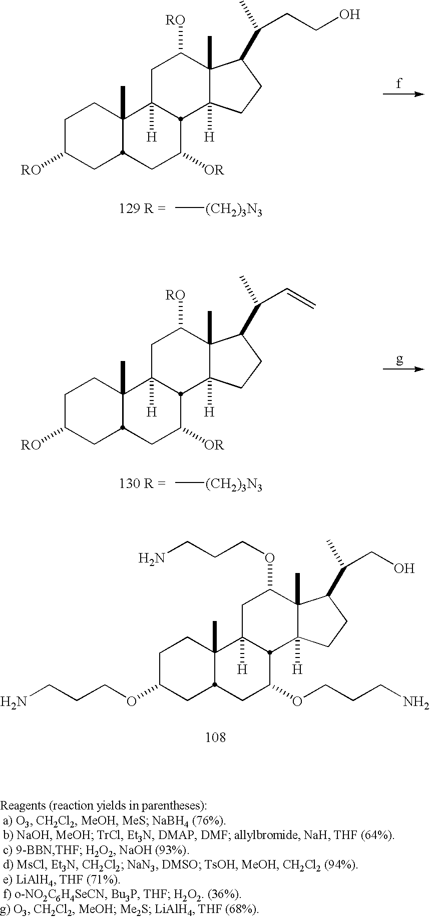

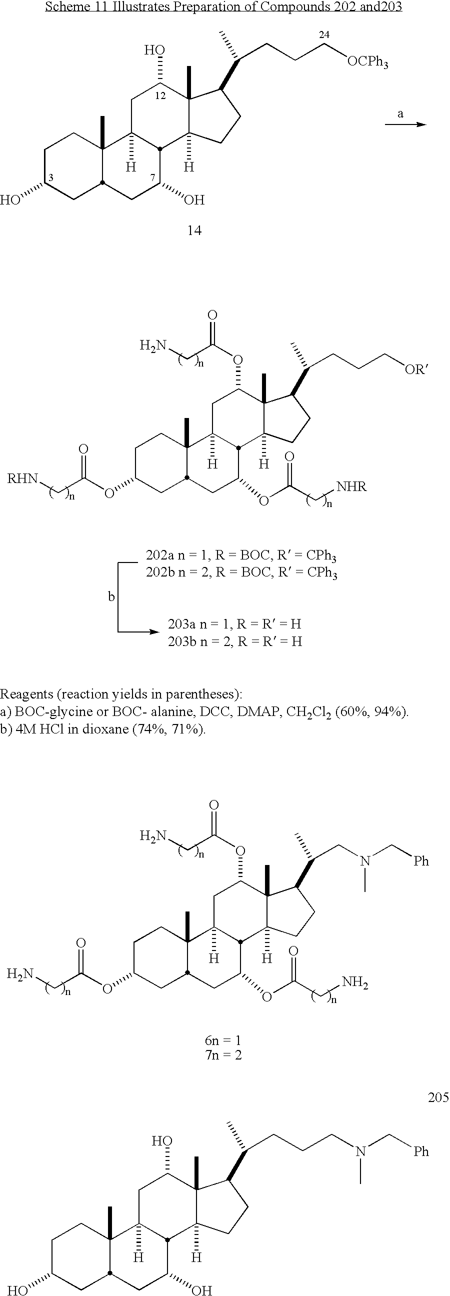

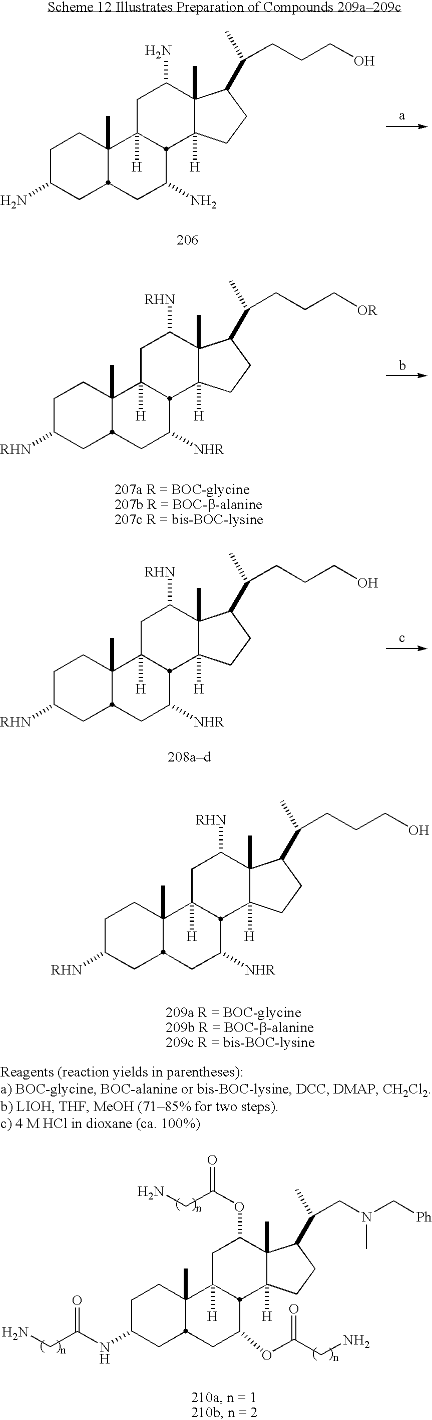

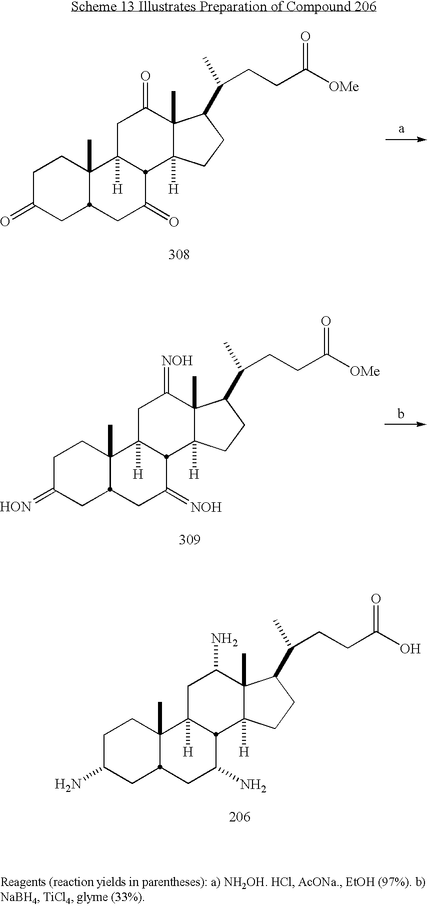

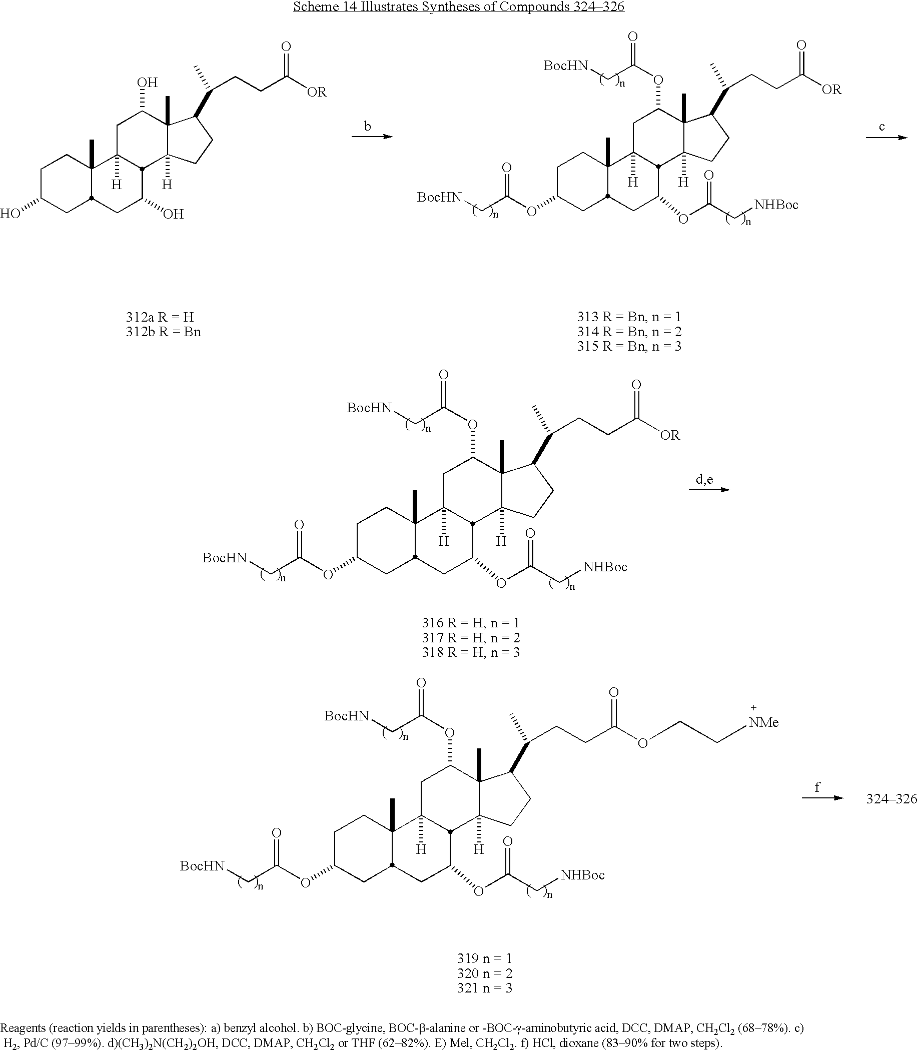

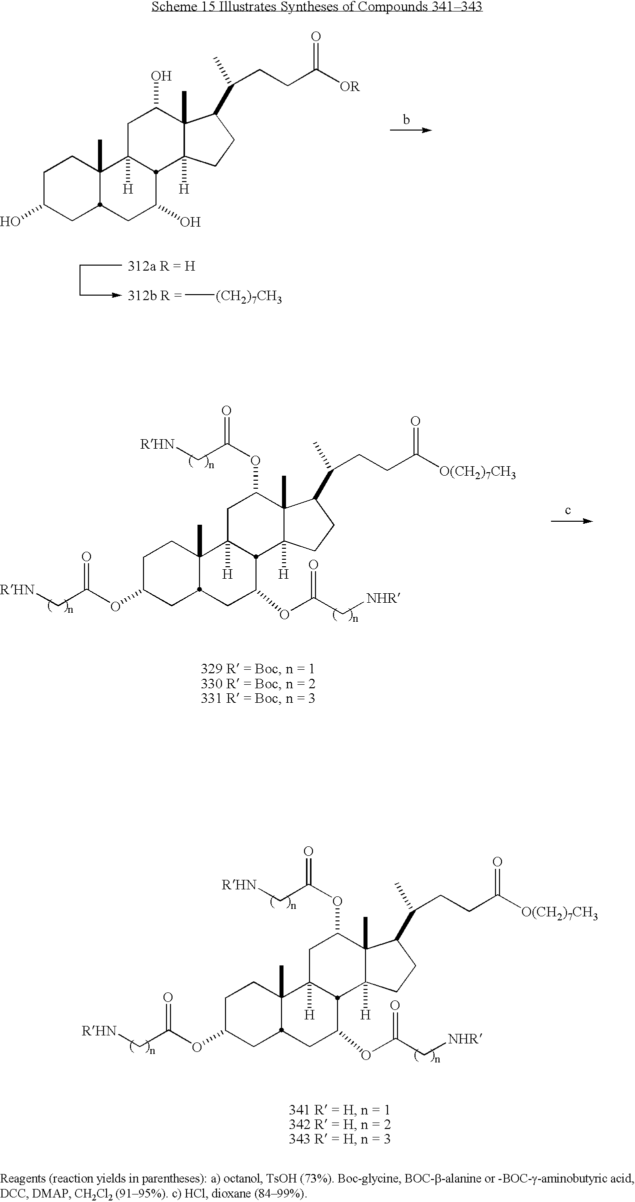

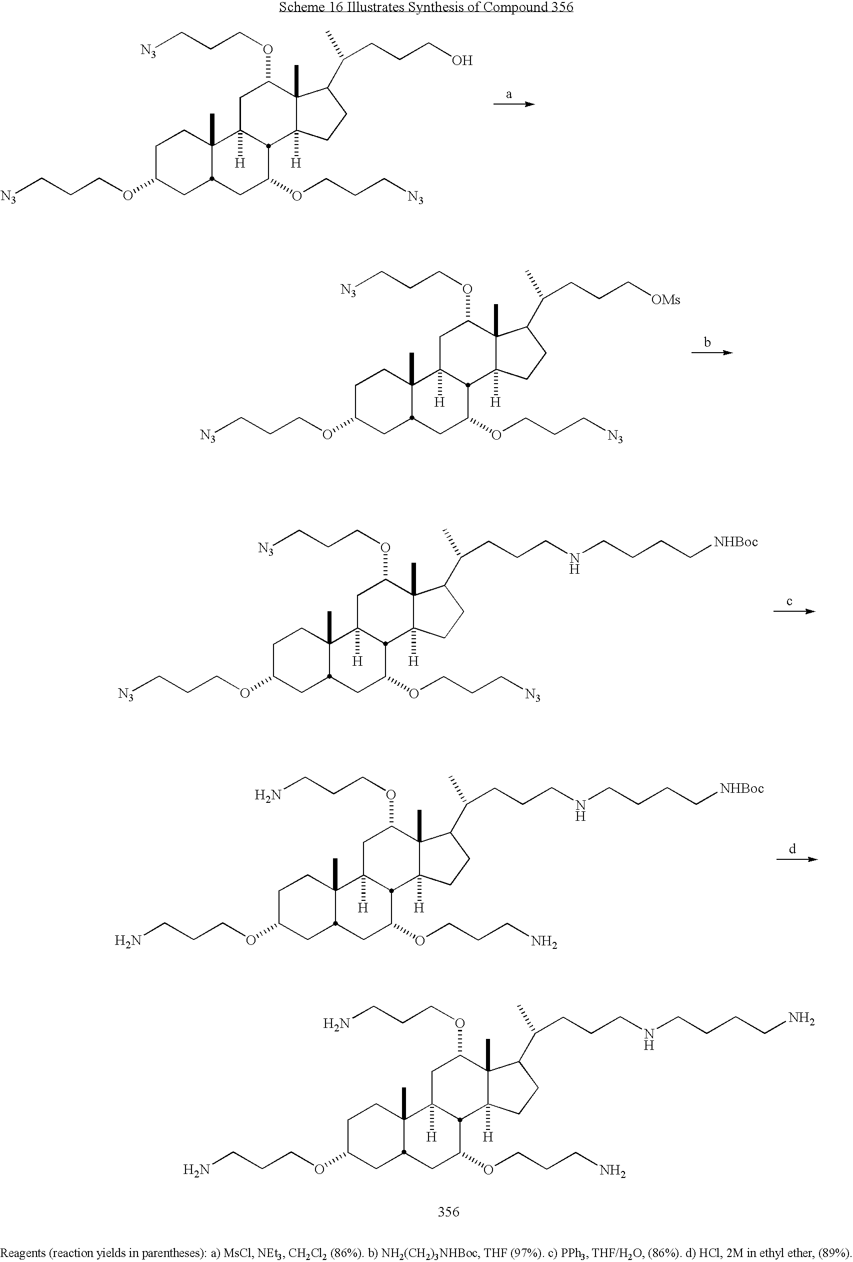

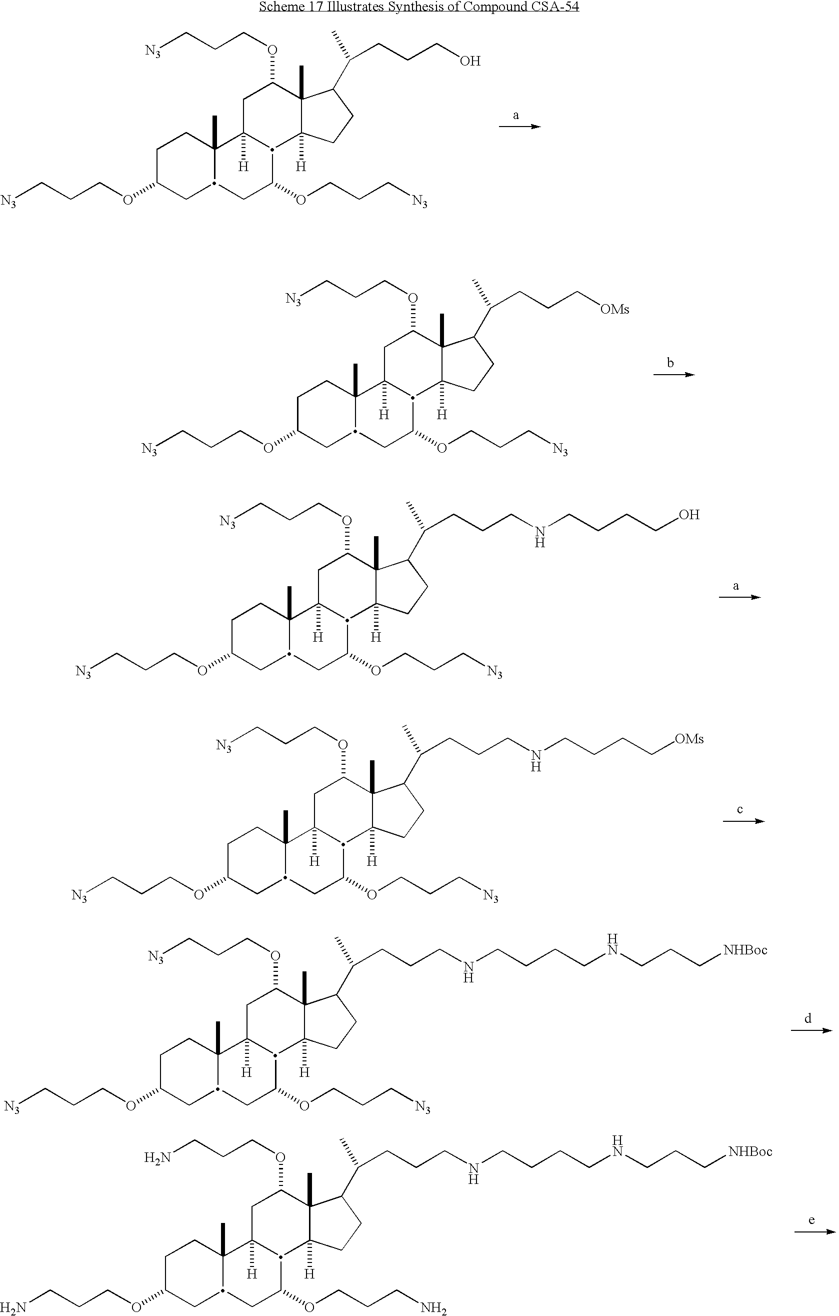

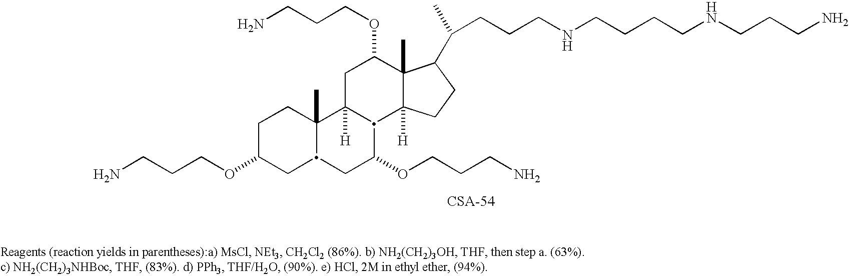

Compounds of the invention and precursors to the compounds according to the invention are available commercially, e.g., from Sigm-Aldrich Co., St. Louis; MO; and Research Plus, Inc., Manasquan, N.J. Other compounds according to the invention can be synthesized according to methods disclosed herein, in U.S. Pat. Nos. 6,350,738; 6,486,148; and 6,767,904, and in the art.

Methods for identifying a candidate agent for treating a subject for an HIV infection or pathogenesis, for decreasing susceptibility of a subject to an HIV infection or pathogenesis and for decreasing, inhibiting, ameliorating or preventing onset, severity, duration, progression, frequency or probability of one or more symptoms associated with HIV infection or pathogenesis, are provided. In one embodiment, a method includes providing a test agent, such as a cationic steroid antimicrobial (CSA); contacting the test agent with HIV and ascertaining whether the test agent inhibits HIV infection or pathogenesis. A test agent identified as inhibiting HIV infection or pathogenesis is a candidate agent for treating a subject for HIV infection or pathogenesis. A test agent identified as inhibiting HIV infection or pathogenesis is also a candidate agent for decreasing susceptibility of a subject to an HIV infection or pathogenesis. A test agent identified as inhibiting HIV infection or pathogenesis is further a candidate agent for decreasing, inhibiting, ameliorating or preventing onset, severity, duration, progression, frequency or probability of one or more symptoms associated with HIV infection or pathogenesis. A test agent identified as inhibiting HIV infection or pathogenesis is additionally a candidate agent for decreasing or preventing an adverse side effect caused by HIV or an HIV treatment. In various aspects, the subject is a mammal (e.g., a primate). For example, a mammal can comprise an animal model for HIV infection or pathogenesis (e.g., SIV infected primate).

Unless otherwise defined, all technical and scientific terms used herein have the same meaning as commonly understood by one of ordinary skill in the art to which this invention belongs. Although methods and materials similar or equivalent to those described herein can be used in the practice or study of the present invention, suitable methods and materials are described herein.

All of the features disclosed herein may be combined in any combination. Each feature disclosed in the specification may be replaced by an alternative feature serving a same, equivalent, or similar purpose. Thus, unless expressly stated otherwise, disclosed features (e.g., compound structures) are an example of a genus of equivalent or similar features.

All applications, publications, patents and other references, GenBank citations and ATCC citations cited herein are incorporated by reference in their entirety. In case of conflict, the specification, including definitions, will control.

As used herein, the singular forms “a”, “and,” and “the” include plural referents unless the context clearly indicates otherwise. Thus, for example, reference to “a compound” includes a plurality of compounds and reference to “an anti-HIV effect, activity or function” can include reference to one or more effects, activities or functions, and so forth.

As used herein, all numerical values or numerical ranges include integers within such ranges and fractions of the values or the integers within ranges unless the context clearly indicates otherwise. Thus, to illustrate, reference to a range of 90−100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. Reference to a range of 0−72 hrs, includes 1, 2, 3, 4, 5, 6, 7 hrs, etc., as well as 1, 2, 3, 4, 5, 6, 7 minutes, etc., and so forth. Reference to a range of 0−72 hrs, includes 1, 2, 3, 4, 5, 6, 7 hrs, etc., as well as 1, 2, 3, 4, 5, 6, 7 minutes, etc., and so forth. Reference to a range of doses, such as 0.1-1 ug/kg, 1-10 ug/kg, 10-25 ug/kg, 25-50 ug/kg, 50-100 ug/kg, 100-500 ug/kg, 500-1,000 ug/kg, 1-5 mg/kg, 5-10 mg/kg, 10-20 mg/kg, 20−50 mg/kg, 50-100 mg/kg, 100-250 mg/kg, 250-500 mg/kg, includes 0.11-0.9 ug/kg, 2-9 ug/kg, 11.5-24.5 ug/kg, 26-49 ug/kg, 55-90 ug/kg, 125-400 ug/kg, 750-800 ug/kg, 1-4.9 mg/kg, 6-9 mg/kg, 11.5-19.5 mg/kg, 21-49 mg/kg, 55-90 mg/kg, 125-200 mg/kg, 275-450.1 mg/kg, etc.

The invention is generally disclosed herein using affirmative language to describe the numerous embodiments. The invention also includes embodiments in which subject matter is excluded, in full or in part, such as substances or materials, method steps and conditions, protocols, or procedures. Thus, even though the invention is generally not expressed herein in terms of what the invention does not include aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. For example, salts, esters, ethers and amides of invention compounds disclosed herein are within the scope of this invention. Accordingly, the following examples are intended to illustrate but not limit the scope of invention described in the claims.

### EXAMPLES

CSA compounds and intermediates were characterized using the following instruments: ¹H and ¹³C NMR spectra were recorded on a Varian Gemini 2000 (200 MHz), Varian Unity 300 (300 MHz), or Varian VXR 500 (500 MHz) spectrometer and are referenced to TMS, residual CHCl₃ (¹H) or CDCl₃ (¹³C), or residual CHD₂OD (¹H), or CD₃OD (¹³C). IR spectra were recorded on a Perkin Elmer 1600 FTIR instrument. Mass spectrometric data were obtained on a JOEL SX 102A spectrometer. THF solvent was dried over Na/benzophenone and CH₂Cl₂ was dried over CaH₂ prior to use. Other reagents and solvents were obtained commercially and were used as received.

### Example 1

This example includes a description of one or more exemplary synthestic procedures for obtaining Compounds 1-5, 13-20 and 22-27.

Compound 13: To a 1 L round-bottom flask were added methyl cholate (30.67 g, 72.7 mmol) in dry THF (600 mL) and LiAlH₄ (4.13 g, 109 mmol). After reflux for 48 hours, saturated aqueous Na₂SO₄ (100 mL) was introduced slowly, and the resulted precipitate was filtered out and washed with hot THF and MeOH. Recrystallization from MeOH gave colorless crystals of 13 (28.0 g, 98% yield). m.p. 236.5-238° C.; IR (KBr) 3375, 2934, 1373, 1081 cm⁻¹; ¹H NMR (CDCl₃/MeOH-d₄, 200 MHz) δ 3.98 (bs, 1H), 3.83 (bs, 1H), 3.60−3.46 (m, 2H), 3.38 (bs, 5H), 2.30−2.10 (m, 2H), 2.05−1.05 (series of multiplets, 22H), 1.03 (bs, 3H), 0.92 (s, 3H), 0.71 (s, 3H); ¹³C NMR (CDCl₃/MeOH-d₄, 50 MHz) δ 73.89, 72.44, 68.99, 63.51, 48.05, 47.12, 42.49, 40.37, 39.99, 36.62, 36.12, 35.58, 35.40, 32.77, 30.69, 30.04, 29.02, 28.43, 27.27, 23.96, 23.08, 18.00, 13.02; HRFAB-MS (thioglycerol+Na⁺ matrix) m/e: ([M+Na]⁺) 417.2992 (55.3%); calcd. 417.2981.

Compound 14: To a round-bottom flask were added 13 (28.2 g, 71.7 mmol) in DMF (300 ml), Et₃ N (20 mL, 143.4 mmol), trityl chloride (25.98 g, 93.2 mmol) and DMAP (0.13 g, 1.07 mmol). The mixture was stirred at 50° C. under N₂ for 30 hours followed by the introduction of water (1000 mL) and extraction with EtOAc (5×200 mL). The combined extracts were washed with water and brine and then dried over MgSO₄. After removal of solvent in vacuo, the residue was purified using SiO₂ chromatography (CH₂Cl₂, Et₂O and MeOH as eluents) to give 14 as a pale yellow solid (31.9 g, 70% yield). m.p. 187° C. (decomposition); IR (KBr) 3405, 2935, 1448, 1075 cm⁻¹; ¹H NMR (CDCl₃, 200 MHz) δ 7.46−7.42 (m, 6H), 7.32−7.17 (m, 9H), 3.97 (bs, 1H), 3.83 (bs, 1H), 3.50−3.38 (m, 1H), 3.01 (bs, 1H), 2.94 (dd, J=14.2, 12.2 Hz, 2H), 2.64 (bs, 1H), 2.51 (bs, 1H), 2.36−2.10 (m, 2H), 2.00−1.05 (series of multiplets, 22H), 0.96 (d, J=5.8 Hz, 3H), 0.87 (s, 3H), 0.64 (s, 3H); ¹³C NMR (CDCl₃, 50 MHz) δ 144.77, 128.93, 127.91, 127.01, 86.43, 73.35, 72.06, 68.66, 64.28, 47.47, 46.53, 41.74, 41.62, 39.64, 35.57, 35.46, 34.91, 34.82, 32.40, 30.55, 28.21, 27.69, 26.80, 26.45, 23.36, 22.59, 17.83, 12.61; HRFAB-MS (thioglycerol+Na⁺ matrix) m/e: ([M+Na]⁺) 659.4069 (100%); calcd. 659.4076.

Compound 15: To a round-bottom flask were added 14 (20.0 g, 31.4 mmol) in dry THF (600 mL) and NaH (60% in mineral oil, 6.3 g, 157.2 mmol). The mixture was refluxed for 30 min under N₂ followed by addition of allyl bromide (27 mL, 314 mmol). After 60 hours of reflux, additional NaH (3 eq.) and allyl bromide (4 eq.) were added. Following another 50 hours of reflux, water (20 mL) was introduced slowly followed by addition of 1% HCl until the aqueous layer became neutral. The mixture was then extracted with ether (3×100 mL) and the combined extracts were washed with water (100 mL) and brine (2×100 mL). The ether solution was dried over anhydrous Na₂SO₄, and after removal of solvent, the residue was purified using SiO₂ chromatography (hexanes and EtOAc/hexanes 1:8 as eluents) to give 15 (22.76 g, 96% yield) as a pale yellow glass. IR (neat) 2930, 1448, 1087 cm⁻¹; ¹H NMR (CDCl₃, 200 MHz) δ 7.48−7.30 (m, 6H), 7.32−7.14 (m, 9H), 6.04−5.80 (m, 3H), 5.36−5.04 (series of multiplets, 6H), 4.14−3.94 (m, 4H), 3.74 (td, J=13.8, 5.8 Hz, 2H), 3.53 (bs, 1H), 3.20−2.94 (m, 3H), 3.31 (bs, 1H), 2.38−1.90 (m, 4H), 1.90−0.96 (series of multiplets, 20H), 0.90 (d, J=5.4 Hz, 3H), 0.89 (s, 3H), 0.64 (s, 3H); ¹³C NMR (CDCl₃, 50 MHz) δ 144.83, 136.27, 136.08, 128.94, 127.90, 126.98, 116.46, 115.70, 86.42, 80.94, 79.29, 74.98, 69.52, 69.39, 68.86, 64.39, 46.51, 46.42, 42.67, 42.14, 39.92, 35.63, 35.51, 35.13, 32.45, 28.98, 28.09, 27.66, 27.57, 26.72, 23.32, 23.11, 17.92, 12.69; HRFAB-MS (thioglycerol+Na⁺ matrix) m/e: ([M+Na]⁺) 779.5013 (86.1%); calcd. 779.5015.

Compound 16: To a three-necked round bottom flask was added 15 (3.34 g, 4.4 mmol) in CH₂Cl₂ (200 mL) and methanol (100 mL). Through the cold solution (−78° C.) ozone was bubbled through until a blue color persisted. Excess ozone was removed with oxygen flow. The mixture was left in a dry ice-acetone bath for an hour. Methyl sulfide (2.4 mL) was added and 15 minutes later, the mixture was treated with NaBH₄ (1.21 g, 32 mmol) in 5% aqueous NaOH solution (10 mL)/methanol (10 mL) and allowed to warm to room temperature. The mixture was washed with brine (3×50 mL), and the combined brine wash was extracted with CH₂Cl₂ (2×50 mL). The organic solution was dried over MgSO₄. After SiO₂ chromatography (MeOH (5%) in CH₂ Cl₂), 3.30 g (95% yield) of 16 was isolated as an oil. IR (neat) 3358, 2934, 1448, 1070 cm⁻¹; ¹H NMR (CDCl₃, 200 MHz) δ 7.50−7.42 (m, 6H), 7.32−7.17 (m, 9H), 3.80−2.96 (series of multiplets, 20H), 2.25−0.96 (series of multiplets, 24H), 0.89 (bs, 6H), 0.65 (s, 3H); ¹³C NMR (CDCl₃, 50 MHz) δ 144.73, 128.88, 127.87, 126.96, 86.38, 81.05, 79.75, 76.59, 70.33, 69.66, 69.30, 64.20, 62.25, 62.16, 62.03, 46.77, 46.36, 42.63, 41.77, 39.60, 35.43, 35.23, 35.05, 34.89, 32.42, 28.91, 27.93, 27.56, 27.15, 26.68, 23.35, 22.98, 22.85, 18.15, 12.60; HRFAB-MS (thioglycerol+Na⁺ matrix) m/e: ([M+Na]⁺) 791.4860 (100%), calcd. 791.4863.

Compound 17: To a round-bottom flask was added 16 (1.17 g, 1.55 mmol) in dry THF (30 mL) under N₂ in ice-bath followed by 9-BBN/THF solution (0.5 M, 10.2 mL, 5.51 mmol). The mixture was stirred at room temperature for 12 hours. Aqueous NaOH (20%) (2 mL) and hydrogen peroxide (30%) (2 mL) were added in sequence. The mixture was refluxed for 1 hour followed by the addition of brine (60 mL) and extraction with EtOAc (4×30 mL). The combined extracts were dried over anhydrous Na₂ SO₄. The product (1.01 g, 80% yield) was obtained as a colorless oil after SiO₂ chromatography (5% MeOH in CH₂ Cl₂). IR (neat) 3396, 2936, 1448, 1365, 1089 cm⁻¹; ¹H NMR(CDCl₃, 200 MHz) δ 7.50−7.42 (m, 6H), 7.34−7.16 (m, 9H), 3.90−3.56 (m, 13H), 3.50 (bs, 1H), 3.40−2.96 (series of multiplets, 6H), 2.30−0.94 (series of multiplets, 30H), 0.90 (s, 3H), 0.88 (d, J=5.4 Hz, 3H), 0.64 (s, 3H); ¹³C NMR(CDCl₃, 50 MHz) δ 144.73, 128.88, 127.85, 126.94, 86.36, 80.52, 78.90, 76.36, 66.82, 66.18, 65.77, 64.22, 61.53, 61.41, 61.34, 46.89, 46.04, 42.60, 41.59, 39.60, 35.37, 35.27, 34.88, 32.75, 32.44, 32.31, 28.82, 27.65, 27.48, 27.13, 26.77, 23.35, 22.74, 22.38, 18.08, 12.48; HRFAB-MS (thioglycerol+Na⁺ matrix) m/e: ([M+Na]⁺) 833.5331 (100%), calcd. 833.5332.

Compound 18: To a round-bottom flask were added 16 (3.30 g, 4.29 mmol) in CH₂Cl₂ (150 mL) and NEt₃ (2.09 mL, 15.01 mmol). The mixture was put in ice-bath under N₂ followed by addition of mesyl chloride (1.10 mL, 14.16 mmol). After 30 minutes, water (30 mL) and brine (200 mL) were added. The CH₂Cl₂ layer was washed with brine (2×50 mL) and dried over anhydrous Na₂SO₄. The combined aqueous mixture was extracted with EtOAc (3×100 mL). The combined extracts were washed with brine and dried over anhydrous Na₂SO₄. The desired product (3.35 g, 78% yield) was isolated as a pale yellow oil after SiO₂ chromatography (EtOAc/hexanes 1:1). IR (neat) 2937, 1448, 1352, 1174, 1120, 924 cm⁻¹; ¹H NMR (CDCl₃, 200 MHz) δ 7.52−7.40 (m, 6H), 7.34−7.20, (m, 9H), 4.42−4.24 (m, 6H), 3.90−3.64 (m, 4H), 3.60−3.30 (m, 4H), 3.24−3.00 (m, 3H), 3.10 (s, 6H), 3.05 (s, 3H), 2.20−1.96 (m, 3H) 1.96−1.60 (m, 8H), 1.60−0.94 (series of multiplets, 13H), 0.91 (bs, 6H), 0.65 (s, 3H); ¹³C NMR(CDCl₃, 50 MHz) δ 114.68, 128.85, 127.85, 126.96, 86.37, 81.37, 79.58, 76.58, 69.95, 69.43, 69.34, 66.52, 66.31, 65.59, 64.11, 46.80, 46.20, 42.65, 41.48, 39.35, 37.82, 37.48, 35.36, 34.92, 34.73, 32.37, 28.66, 28.01, 27.44, 27.03, 26.72, 23.17, 22.91, 22.72, 18.13, 12.50; HRFAB-MS (thioglycerol+Na⁺ matrix) m/e: ([M+Na]⁺) 1205.4176 (81.5%), calcd. 1205.4189.

Compound 19: To a round-bottom flask were added 17 (1.01 g, 1.25 mmol) in CH₂Cl₂ (50 mL) and NEt₃ (0.608 mL, 4.36 mmol). The mixture was put in ice-bath under N₂ followed by addition of mesyl chloride (0.318 mL, 4.11 mmol). After 30 minutes, water (10 mL) and then brine (80 mL) were added. The CH₂ Cl₂ layer was washed with brine (2×20 mL) and dried over anhydrous Na₂SO₄. The combined aqueous mixture was extracted with EtOAc (3×40 mL). The combined extracts were washed with brine and dried over anhydrous Na₂ SO₄. The desired product (1.07 g, 82%) was isolated as a pale yellowish oil after SiO₂ chromatography (EtOAc/hexanes 1:1). IR (neat) 2938, 1356, 1176, 1112 cm⁻¹; ¹H NMR (CDCl₃, 300 MHz) δ 7.46−7.43, (m, 6H), 7.32−7.22 (m, 9H), 4.40−4.31 (m, 6H), 3.72−3.64 (m, 2H), 3.55 (dd, J=6.3, 5.8 Hz, 2H), 3.51 (bs, 1H), 3.32−3.14 (m, 3H), 3.14−2.92 (m, 3H), 3.01 (s, 3H), 3.01 (s, 3H), 3.00 (s, 3H), 2.10−1.92 (m, 10H), 1.92−1.58 (m, 8H), 1.56−0.92 (series of multiplets, 12H), 0.90 (s, 3H), 0.89 (d, J=5.4 Hz, 3H), 0.64 (s, 3H); ¹³C NMR(CDCl₃, 75 MHz) δ 144.67, 128.85, 127.85, 126.96, 86.42, 81.06, 79.83, 76.81, 68.12, 68.06, 68.02, 64.26, 64.06, 63.42, 46.76, 46.38, 42.73, 41.87, 39.73, 37.44, 37.32, 37.29, 35.52, 35.48, 35.32, 35.06, 32.53, 30.55, 30.28, 30.02, 29.15, 27.96, 27.69, 27.61, 26.75, 23.52, 23.02, 18.17, 12.64; HRFAB-MS (thioglycerol+Na⁺ matrix) m/e: ([M+Na]⁺) 1067.4672 (100%), calcd. 1067.4659.

Compound 20: To a round-bottom flask were added 18 (1.50 g, 1.50 mmol) in dry DMSO (20 mL) and NaN₃ (0.976 g, 15 mmol). The mixture was heated to 80° C. and stirred under N₂ overnight then diluted with water (100 mL). The resulted aqueous mixture was extracted with EtOAc (3×50 mL), and the combined extracts washed with brine and dried over anhydrous Na₂ SO₄. The desired product (0.83 g, 66% yield) was isolated as a clear glass after SiO₂ chromatography (EtOAc/hexanes 1:5). IR (neat) 2935, 2106, 1448, 1302, 1114 cm⁻¹; ¹H NMR (CDCl₃, 200 MHz) δ 7.50−7.42 (m, 6H), 7.36−7.20 (m, 9H), 3.84−3.70 (m, 2H), 3.65 (t, J=4.9 Hz, 2H), 3.55 (bs, 1H), 3.44−3.08 (m, 10H), 3.02 (t, J=6.4 Hz, 2H), 2.38−0.96 (series of multiplets, 24H), 0.92 (d, J=5.6 Hz, 3H), 0.91 (s, 3H), 0.65 (s, 3H); ¹³C NMR (CDCl₃, 50 MHz) δ 114.84, 128.97, 127.92, 126.99, 86.42, 81.24, 80.12, 76.59, 67.84, 67.29, 66.66, 64.36, 51.67, 51.44, 51.18, 46.53, 46.23, 42.21, 41.93, 39.73, 35.66, 35.36, 35.06, 34.78, 32.40, 28.95, 27.76, 27.39, 26.87, 23.45, 22.98, 22.92, 17.98, 12.53; HRFAB-MS (thioglycerol+Na⁺ matrix) m/e: ([M+Na]⁺) 866.5040 (100%), calcd. 866.5057.

Compound 22: To a round-bottom flask were added 20 (830 mg, 0.984 mmol) in MeOH (30 mL) and CH₂ Cl₂ (30 mL) and p-toluenesulfonic acid (9.35 mg, 0.0492 mmol). The solution was stirred at room temperature for 2.5 hours then saturated aqueous NaHCO₃ (10 mL) was introduced. Brine (30 mL) was added, and the mixture was extracted with EtOAc (4×20 mL). The combined extracts were dried over anhydrous Na₂ SO₄. The desired product (0.564 g, 95% yield) was isolated as a pale yellowish oil after SiO₂ chromatography (EtOAc/hexanes 1:2). IR (neat) 3410, 2934, 2106, 1301, 1112 cm¹; ¹H NMR (CDCl₃, 200 MHz) δ 3.80−3.54 (m, 7H), 3.44−3.20 (m, 10H), 2.35−0.96 (series of multiplets, 24H), 0.95 (d, J=6.4 Hz, 3H), 0.92 (s, 3H), 0.68 (s, 3H); ¹³C NMR (CDCl₃, 50 MHz) δ 81.10, 80.01, 76.60, 67.75, 67.16, 66.56, 63.63, 51.57, 51.34, 51.06, 46.29, 46.12, 42.12, 41.81, 39.60, 35.55, 35.23, 34.94, 34.66, 31.75, 29.48, 28.81, 27.72, 27.66, 27.29, 23.32, 22.86, 22.80, 17.85, 12.39; HRFAB-MS (thioglycerol+Na⁺ matrix) m/e: ([M+Na]⁺) 624.3965 (100%), calcd. 624.3962.

Compound 23: To a round-bottom flask were added 19 (1.07 g, 1.025 mmol) and NaN₃ (0.666 g, 10.25 mmol) followed the introduction of dry DMSO (15 mL). The mixture was heated up to 80° C. under N₂ overnight. After the addition of H₂ O (100 mL), the mixture was extracted with EtOAc (4×40 mL) and the combined extracts were washed with brine (2×50 mL) and dried over anhydrous Na₂ SO₄. After removal of solvent, the residue was dissolved in MeOH (15 mL) and CH₂ Cl₂ (15 mL) followed by the addition of catalytic amount of p-toluenesulfonic acid (9.75 mg, 0.051 mmol). The solution was stirred at room temperature for 2.5 hours before the addition of saturated NaHCO₃ solution (15 mL). After the addition of brine (60 mL), the mixture was extracted with EtOAc (5×30 mL). The combined extracts were washed with brine (50 mL) and dried over anhydrous Na₂SO₄. The desired product (0.617 g, 94% yield for two steps) was obtained as a yellowish oil after SiO₂ chromatography (EtOAc/hexanes 1:2). IR (neat) 3426, 2928, 2094, 1456, 1263, 1107 cm⁻¹; ¹H NMR (CDCl₃, 300 MHz) δ 3.68−3.56 (m, 3H), 3.56−3.34 (series of multiplets, 10H), 3.28−3.00 (series of multiplets, 4H), 2.20−2.00 (m, 3H), 1.98−1.55 (series of multiplets, 15H), 1.55−0.96 (series of multiplets, 13H), 0.92 (d, J=6.6 Hz, 3H), 0.89 (s, 3H), 0.66 (s, 3H); ¹³C NMR (CDCl₃, 75 MHz) δ 80.63, 79.79, 76.04, 64.99, 64.45, 64.30, 63.72, 49.01, 48.94, 48.74, 46.49, 46.39, 42.70, 41.98, 39.80, 35.65, 35.42, 35.28, 35.08, 31.99, 29.78, 29.75, 29.70, 29.49, 29.06, 27.87, 27.79, 27.65, 23.53, 23.04, 22.85, 18.05, 12.59; HRFAB-MS (thioglycerol+Na matrix) m/e: ([M+Na]⁺) 666.4415 (100%), calcd. 666.4431.

Compound 24: To a round-bottom flask were added 22 (0.564 g, 0.938 mmol) in CH₂Cl₂ (30 mL) and NEt₃ (0.20 mL, 1.40 mmol). The mixture was put in ice-bath under N₂ followed by addition of mesyl chloride (0.087 mL, 1.13 mmol). After 30 minutes, water (20 mL) and brine (100 mL) were added. The CH₂ Cl₂ layer was washed with brine (2×20 mL) and dried over anhydrous Na₂ SO₄. The combined aqueous mixture was extracted with EtOAc (3×30 mL). The combined extracts were washed with brine and dried over anhydrous Na₂ SO₄. The desired product (0.634 g, 99% yield) was isolated as a pale yellowish oil after SiO₂ chromatography (EtOAc/hexanes 1:2). IR (neat) 2935, 2106, 1356, 1175, 1113 cm¹; ¹H NMR (CDCl₃, 300 MHz) δ 4.20 (t, J=6.8 Hz, 2H), 3.80−3.75 (m, 1H), 3.70−3.64 (m, 3H), 3.55 (bs, 1H), 3.44−3.01 (m, 10H), 3.00 (s, 3H), 2.32−2.17 (m, 3H), 2.06−2.03 (m, 1H), 1.90−0.88 (series of multiplets, 20H), 0.95 (d, J=6.6 Hz, 3H), 0.91 (s, 3H), 0.68 (s, 3H); ¹³C NMR (CDCl₃, 75 MHz) δ 80.90, 79.86, 76.43, 70.78, 67.64, 66.99, 66.48, 51.50, 51.26, 50.97, 46.05, 45.96, 42.08, 41.71, 39.51, 37.33, 35.15, 34.86, 34.60, 31.34, 28.73, 27.62, 27.59, 27.51, 25.68, 23.22, 22.80, 22.70, 17.62, 12.33; HRFAB-MS (thioglycerol+Na⁺ matrix) m/e: ([M+Na]⁺) 702.3741 (100%), calcd. 702.3737.

Compound 25: To a round-bottom flask were added 23 (0.617 g, 0.96 mmol) in CH₂ Cl₂ (30 mL) and NEt₃ (0.20 mL, 1.44 mmol). The mixture was put in ice-bath under N₂ followed by addition of mesyl chloride (0.089 mL, 1.15 mmol). After 30 minutes, water (20 mL) and brine (120 mL) were added. The CH₂ Cl₂ layer was washed with brine (2×20 mL) and dried over anhydrous Na₂ SO₄. The combined aqueous mixture was extracted with EtOAc (3×30 mL). The combined extracts were washed with brine and dried over anhydrous Na₂ SO₄. The desired product (0.676 g, 97% yield) was isolated as a pale yellowish oil after removal of solvent. IR (neat) 2934, 2094, 1454, 1360, 1174, 1112 cm⁻¹; ¹H NMR (CDCl₃, 300 MHz) δ 4.17 (t, J=6.6 Hz, 2H), 3.65−3.28 (series of multiplets, 11H), 3.64−3.00 (series of multiplets, 4H), 2.97 (s, 3H), 2.18−1.96 (series of multiplets, 16H), 1.54−0.94 (series of multiplets, 11H), 0.89 (d, J=6.6 Hz, 3H), 0.86 (s, 3H), 0.63 (s, 3H); ¹³C NMR (CDCl₃, 75 MHz) δ 80.47, 79.67, 75.92, 70.84, 64.90, 64.37, 64.17, 48.90, 48.86, 48.66, 46.32, 46.26, 42.63, 41.87, 39.70, 37.39, 35.34, 35.28, 35.20, 34.99, 31.61, 29.68, 29.60, 28.96, 27.78, 27.68, 27.57, 25.79, 23.41, 22.95, 22.74, 17.82, 12.50; HRFAB-MS (thioglycerol matrix) m/e: ([M+H]⁺) 722.4385 (22.1%), calcd. 722.4387.

Compound 26: To a 50 mL round-bottom flask was added 24 (0.634 g, 0.936 mmol) and N-benzylmethylamine (2 mL). The mixture was heated under N₂ at 80° C. overnight. Excess N-benzylmethylamine was removed under vacuum, and the residue was subjected to SiO₂ chromatography (EtOAc/hexanes 1:2). The desired product (0.6236 g, 95% yield) was isolated as a pale yellow oil. IR (neat) 2935, 2106, 1452, 1302, 1116 cm⁻¹; ¹H NMR (CDCl₃, 200 MHz) δ 7.32−7.24 (m, 5H), 3.80−3.76 (m, 1H), 3.70−3.60 (m, 3H), 3.54 (bs, 1H), 3.47 (s, 2H), 3.42−3.10 (m, 10H), 2.38−2.05 (m, 5H), 2.17 (s, 3H), 2.02−0.88 (series of multiplets, 21H), 0.93 (d, J=7.0 Hz, 3H), 0.91 (s, 3H), 0.66 (s, 3H); ¹³C NMR (CDCl₃, 50 MHz) δ 139.60, 129.34, 128.38, 127.02, 81.22, 80.10, 76.71, 67.85, 67.29, 66.65, 62.45, 58.38, 51.65, 51.44, 51.16, 46.50, 46.21, 42.40, 42.20, 41.93, 39.72, 35.80, 35.34, 35.05, 34.76, 33.65, 28.93, 27082, 27.75, 27.38, 24.10, 23.45, 22.98, 22.91, 18.05, 12.50; HRFAB-MS (thioglycerol+Na⁺ matrix) m/e: ([M−H]⁺) 703.4748 (90.2%), calcd. 703.4772; ([M+H]⁺) 705.4911 (100%), calcd. 705.4928; ([M+Na]⁺) 727.4767 (1.5%), calcd. 727.4748.

Compound 27: To a 50 mL round-bottom flask was added 25 (0.676 g, 0.937 mmol) and N-benzylmethylamine (2 mL). The mixture was heated under N₂ at 80° C. overnight. Excess N-benzylmethylamine was removed under vacuum and the residue was subjected to SiO₂ chromatography (EtOAc/hexanes 1:2). The desired product (0.672 g, 96% yield) was isolated as a pale yellow oil. IR (neat) 2934, 2096, 1452, 1283, 107 cm⁻¹; ¹H NMR (CDCl₃, 300 MHz) δ 7.34−7.20 (m, 5H), 3.68−3.37 (series of multiplets, 13H), 3.28−3.04 (m, 4H), 2.33 (t, J=7.0 Hz, 2H), 2.18 (s, 3H), 2.20−2.00 (m, 3H), 1.96−1.56 (series of multiplets, 14H), 1.54−1.12 (m, 10H), 1.10−0.96 (m, 3H), 0.91 (d, J=8.7 Hz, 3H), 0.89 (s, 3H), 0.65 (s, 3H); ¹³C NMR (CDCl₃, 75 MHz) δ 139.48, 129.23, 128.30, 26.96, 80.66, 79.81, 76.08, 65.00, 64.46, 64.34, 62.50, 58.37, 49.02, 48.95, 48.75, 46.65, 46.40, 42.69, 42.43, 42.00, 39.83, 35.86, 35.45, 35.30, 35.10, 33.83, 29.81, 29.78, 29.72, 29.09, 27.88, 27.81, 27.66, 24.19, 23.57, 23.06, 22.87, 18.15, 12.62; HRFAB-MS (thioglycerol matrix) m/e: ([M+H]⁺) 747.5406 (77.2%), calcd. 747.5398.

Compound 1: To a round-bottom flask were added 26 (0.684 g, 0.971 mmol) in dry THF (30 mL) and LiAlH₄ (113.7 mg, 3.0 mmol) under N₂. The mixture was stirred at room temperature for 12 hours, and then Na₂SO₄.10 H₂O powder (10 g) was added slowly. After the grey color disappeared, the mixture was filtered through Celite and washed with dry THF. The product (0.581 g, 95% yield) was obtained as a colorless glass. IR (neat) 3372, 2937, 1558, 1455, 1362, 1102 cm¹; ¹H NMR (CDCl₃, 300 MHz) δ 7.34−7.20 (m, 5H), 3.68−3.48 (m, 5H), 3.47 (s, 2H), 3.29 (bs, 1H), 3.22−3.00 (m, 3H), 2.96−2.80 (m, 6H), 2.32 (t, J=6.8, 5.4 Hz, 2H), 2.17 (s, 3H), 2.20−2.00 (m, 3H), 1.96−0.96 (series of multiplets, 27H), 0.93 (d, J=6.8 Hz, 3H), 0.90, (s, 3H), 0.67 (s, 3H); ¹³C NMR (CDCl₃, 75 MHz) δ 139.50, 129.22, 128.31, 126.96, 80.76, 79.85, 76.10, 70.90, 70.33, 70.24, 62.48, 58.27, 46.55, 46.45, 42.72, 42.58, 42.33, 41.99, 39.77, 35.78, 35.37, 35.01, 33.73, 29.07, 27.95, 27.71, 24.06, 23.46, 22.99, 18.14, 12.55; HRFAB-MS (thioglycerol matrix) m/e: ([M+H]⁺) 627.5211 (100%), calcd. 627.5213.

HCl salt of compound 1: Compound 1 was dissolved in a minimum amount of CH₂ Cl₂ and excess HCl in ether was added. Solvent and excess HCl were removed in vacuo and a noncrystalline white powder was obtained. ¹H NMR (methanol-d4/15% (CDCl₃, 300 MHz) δ 7.61−7.57 (m, 2H), 7.50−7.48 (m, 3H), 4.84 (bs, 10H), 4.45 (bs, 1H), 4.30 (bs, 1H), 3.96−3.82 (m, 2H), 3.78−3.69 (m, 2H), 3.66 (bs, 1H), 3.59−3.32 (series of multiplets, 4H), 3.28−3.02 (m, 8H), 2.81 (s, 3H), 2.36−2.15 (m, 4H), 2.02−1.68 (m, 8H), 1.64−0.90 (series of multiplets, 12H), 1.01 (d, J=6.35 Hz, 3H), 0.96 (s, 3H), 0.73 (s, 3H); ¹³C NMR (methanol-d4/15% (CDCl₃, 75 MHz) δ 132.31, 131.20, 130.92, 130.40, 83.13, 81.09, 78.48, 65.54, 64.98, 64.11, 60.87, 57.66, 47.51, 46.91, 43.52, 43.00, 41.38, 41.19, 41.16, 40.75, 40.30, 36.37, 36.08, 36.00, 35.96, 33.77, 29.68, 29.34, 28.65, 28.37, 24.42, 24.25, 23.33, 21.51, 18.80, 13.04.

Compound 2: To a round-bottom flask were added 27 (0.82 g, 1.10 mmol) in dry THF (150 mL) and LiAlH₄ (125 mg, 3.30 mmol) under N₂. The mixture was stirred at room temperature for 12 hours and Na₂ SO₄.10 H₂ O powder (10 g) was added slowly. After the grey color disappeared, the mixture was filtered through a cotton plug and washed with dry THF. THF was removed in vacuo and the residue dissolved in CH₂ Cl₂ (50 mL). After filtration, the desired product was obtained as a colorless glass (0.73 g, 99% yield). IR (neat) 3362, 2936, 2862, 2786, 1576, 1466, 1363, 1103 cm⁻¹; ¹H NMR (CDCl₃, 300 MHz) δ 7.32−7.23 (m, 5H), 3.67−3.63 (m, 1H), 3.60−3.57 (m, 1H), 3.53 (t, J=6.4 Hz, 2H), 3.47 (s, 2H), 3.46 (bs, 1H), 3.24−3.17 (m, 2H), 3.12−2.99 (m, 2H), 2.83−2.74 (series of multiplets, 6H), 2.30 (t, J=7.3 Hz, 2H), 2.15 (s, 3H), 2.20−2.00 (m, 3H), 1.95−1.51 (series of multiplets, 20H), 1.51−1.08, (series of multiplets, 10H), 1.06−0.80 (m, 3H), 0.87 (d, J=8.1 Hz, 3H), 0.86 (s, 3H), 0.61 (s, 3H); ¹³C NMR (CDCl₃, 75 MHz). 139.35, 129.16, 128.22, 126.88, 80.44, 79.29, 75.96, 66.70, 66.52, 66.12, 62.45, 58.26, 46.76, 46.27, 42.69, 42.41, 42.02, 40.68, 40.10, 40.02, 39.82, 35.84, 35.47, 35.30, 35.06, 34.15, 34.09, 34.03, 33.80, 28.96, 27.93, 27.75, 27.71, 24.32, 23.53, 23.03, 22.75, 18.17, 12.58; HRFAB-MS (thioglycerol+Na⁺ matrix) m/e: ([M+Na]⁺) 691.5504 (38.5%), calcd. 691.5502.

HCl salt of compound 2: Compound 2 was dissolved in a minimum amount of CH₂ Cl₂ and excess HCl in ether was added. Removal of the solvent and excess HCl gave a noncrystalline white powder. ¹H NMR (methanol-d₄/15% (CDCl₃, 300 MHz) δ 7.60−7.59 (m, 2H), 7.50−7.47 (m, 3H), 4.82 (bs, 10H), 4.43 (bs, 1H), 4.32 (bs, 1H), 3.85−3.79 (m, 1H), 3.75−3.68 (m, 1H), 3.64 (t, J=5.74 Hz, 2H), 3.57 (bs, 1H), 3.36−3.28 (m, 2H), 3.25−3.00 (series of multiplets, 10H), 2.82 (s, 3H), 2.14−1.68 (series of multiplets, 19H), 1.65−1.15 (series of multiplets, 11H), 0.98 (d, J=6.6 Hz, 3H), 0.95 (s, 3H), 0.72 (s, 3H); ¹³C NMR (methanol-d4/15% (CDCl₃, 75 MHz) δ 132.21, 131.10, 130.58, 130.28, 81.96, 80.72, 77.60, 66.84, 66.58, 66.12, 61.03, 57.60, 44.16, 42.77, 40.62, 39.57, 39.43, 36.28, 36.03, 35.96, 35.78, 33.65, 29.48, 29.27, 29.11, 29.01, 28.61, 28.56, 28.35, 24.25, 23.56, 23.30, 21.17, 18.64, 12.90.

Compound 4: A suspension of 1 (79.1 mg, 0.126 mmol) and aminoiminomethanesulfonic acid (50.15 mg, 0.404 mmol) in methanol and chloroform was stirred at room temperature for 24 hours, and the suspension became clear. An ether solution of HCl (1 M, 1 mL) was added followed by the removal of solvent with N₂ flow. The residue was dissolved in H₂O (5 mL) followed by the addition of 20% aqueous NaOH (0.5 mL). The resulting cloudy mixture was extracted with CH₂Cl₂ (4×5 mL). The combined extracts were dried over anhydrous Na₂SO₄. Removal of solvent gave the desired product (90 mg, 95%) as white powder. m.p. 111-112° C. IR (neat) 3316, 2937, 1667, 1650, 1556, 1454, 1348, 1102 cm⁻¹; ¹H NMR (5% methanol-d4/CDCl₃, 300 MHz) δ 7.26−7.22 (m, 5H), 4.37 (bs, 3H), 3.71−3.51 (series of multiplets, 5H), 3.44 (s, 2H), 3.39−3.10 (series of multiplets, 10H), 2.27 (t, J=6.83 Hz, 2H), 2.13 (s, 3H), 2.02−0.94 (series of multiplets, 33H), 0.85 (d, J=5.62 Hz, 3H), 0.84 (s, 3H), 0.61 (s, 3H); ¹³C NMR (5% methanol-d4/CDCl₃, 75 MHz) δ 158.54, 158.48, 158.43, 138.27, 129.47, 128.32, 127.19, 81.89, 80.30, 77.34, 69.02, 68.46, 67.21, 62.36, 58.00, 47.36, 46.18, 43.26, 43.00, 42.73, 42.18, 41.48, 39.32, 35.55, 34.97, 34.89, 34.67, 33.63, 28.93, 28.28, 27.53, 27.16, 23.96, 23.28, 23.16, 22.77, 18.36, 12.58; HRFAB-MS (thioglycerol+Na⁺ matrix) m/e: ([M+H]⁺) 753.5858 (100%), calcd. 753.5867.

HCl salt of compound 4: Compound 4 was dissolved in minimum amount of CH₂Cl₂ and MeOH followed by addition of excess HCl in ether. The solvent was removed by N₂ flow, and the residue was subjected to high vacuum overnight. The desired product was obtained as noncrystalline white powder. ¹H NMR (methanol-d4/20% (CDCl₃, 300 MHz) δ 7.58 (bs, 2H), 7.50−7.48 (m, 3H), 4.76 (bs, 13H), 4.45 (d, J=12.9 Hz, 1H), 4.27 (dd, 1H, J=12.9, 5.4 Hz), 3.82−3.00 (series of multiplets, 17H), 2.81−2.80 (m, 3H), 2.20−1.02 (series of multiplets, 27H), 0.98 (d, J=6.59 Hz, 3H), 0.95 (s, 3H), 0.72 (s, 3H); ¹³C NMR (methanol-d4/20% CDCl₃, 75 MHz) δ 158.88, 158.72, 132.00, 131.96, 130.98, 130.15, 82.51, 81.07, 78.05, 68.50, 68.02, 67.94, 67.10, 60.87, 60.53, 57.38, 47.16, 46.91, 43.91, 43.11, 43.01, 42.91, 42.55, 40.28, 39.88, 39.95, 35.90, 35.73, 35.64, 33.53, 29.18, 28.35, 27.99, 24.02, 23.30, 21.35, 18.52, 18.44, 13.06.

Compound 5: A suspension of 2 (113 mg, 0.169 mmol) and aminoiminomethanesulfonic acid (67.1 mg, 0.541 mmol) in methanol and chloroform was stirred at room temperature for 24 hours. HCl in ether (1 M, 1 mL) was added followed by the removal of solvent with N₂ flow. The residue was subject to high vacuum overnight and dissolved in H₂O (5 mL) followed by the addition of 20% NaOH solution (1.0 mL). The resulting mixture was extracted with CH₂ Cl₂ (5×5 mL). The combined extracts were dried over anhydrous Na₂ SO₄. Removal of solvent gave desired the product (90 mg, 95% yield) as a white solid. m.p. 102-104° C. IR (neat) 3332, 3155, 2939, 2863, 1667, 1651, 1558, 1456, 1350, 1100 cm⁻¹; ¹H NMR (5% methanol-d4/CDCl₃, 300 MHz) δ 7.35−7.24 (m, 5H), 3.75−3.64 (m, 1H), 3.57 (bs, 5H), 3.50 (s, 2H), 3.53−3.46 (m, 1H), 3.40−3.10 (series of multiplets, 14H), 2.34 (t, J=7.31 Hz, 2H), 2.19 (s, 3H), 2.13−0.96 (series of multiplets, 36H), 0.91 (bs, 6H), 0.66 (s, 3H); ¹³C NMR (5% methanol-d4/CDCl₃, 75 MHz) δ 157.49, 157.31, 157.23, 138.20, 129.52, 128.34, 127.23, 81.17, 79.19, 76.42, 65.63, 65.03, 64.70, 62.36, 58.02, 47.23, 46.24, 42.89, 42.18, 41.45, 39.45, 39.40, 39.30, 38.71, 35.61, 35.55, 35.02, 34.82, 33.69, 29.87, 29.59, 29.42, 28.84, 27.96, 27.56, 23.95, 23.40, 22.82, 22.64, 18.28, 12.54; HRFAB-MS (thioglycerol+Na⁺ matrix) m/e: ([M+H]⁺) 795.6356 (84.3%), calcd. 795.6337.

HCl salt of compound 5: Compound 5 was dissolved in minimum amount of CH₂ Cl₂ and MeOH followed by the addition of excess HCl in ether. The solvent and excess HCl were removed by N₂ flow and the residue was subject to high vacuum overnight. The desired product was obtained as noncrystalline white powder. ¹H NMR (methanol-d4/10% CDCl₃, 300 MHz) δ 7.62−7.54 (m, 2H), 7.48−7.44 (m, 3H), 4.84 (bs, 16H), 4.46 (d, J=12.7 Hz, 1H), 4.26 (dd, J=12.7, 3.42 Hz, 1H), 3.78−3.56 (series of multiplets, 5H), 3.38−3.05 (series of multiplets, 13H), 2.80 (d, 3H), 2.19−2.04 (m, 3H), 2.02−1.04 (series of multiplets, 30H), 0.98 (d, J=6.35 Hz, 3H), 0.95 (s, 3H), 0.72 (s, 3H); ¹³C NMR (methanol-d4/10% CDCl₃, 75 MHz) δ 158.75, 158.67, 132.32, 131.24, 130.83, 130.43, 82.49, 81.02, 77.60, 66.47, 65.93, 61.19, 60.85, 57.69, 47.79, 47.60, 44.29, 43.07, 40.86, 40.42, 40.19, 40.09, 39.76, 36.68, 36.50, 36.15, 35.94, 33.91, 30.75, 30.46, 29.74, 29.33, 28.71, 24.41, 24.03, 23.38, 22.21, 22.16, 18.59, 18.52, 13.09.

Compound CSA-26 was synthesized according to Scheme 1 and Example 1 using 7-deoxycholic acid in place of cholic acid and methyl cholate.

### Example 2

This example includes a description of one or more exemplary synthestic procedures for obtaining Compounds 3, 28 and 29.

Compound 28: A suspension of 19 (0.641 g, 0.614 mmol) and KCN (0.40 g, 6.14 mmol) in anhydrous DMSO (5 mL) was stirred under N₂ at 80° C. overnight followed by the addition of H₂O (50 mL). The aqueous mixture was extracted with EtOAc (4×20 mL). The combined extracts were washed with brine once, dried over anhydrous Na₂ SO₄ and concentrated in vacuo. The residue was dissolved in CH₂ Cl₂ (3 mL) and MeOH (3 mL) and catalytic amount of p-toluenesulfonic acid (5.84 mg, 0.03 mmol) was added. The solution was stirred at room temperature for 3 hours before the introduction of saturated NaHCO₃ solution (10 mL). After the addition of brine (60 mL), the mixture was extracted with EtOAc (4×30 mL). The combined extracts were washed with brine once and dried over anhydrous Na₂ SO₄ and concentrated. The residue afforded the desired product (0.342 g, 92% yield) as pale yellowish oil after column chromatography (silica gel, EtOAc/hexanes 2:1). IR (neat) 3479, 2936, 2864, 2249, 1456, 1445, 1366, 1348, 1108 cm⁻¹; ¹H NMR (CDCl₃, 300 MHz) δ 3.76−3.53 (m, 7H), 3.32−3.06 (series of multiplets, 4H), 2.57−2.46 (m, 6H), 2.13−1.00 (series of multiplets, 31H), 0.93 (d, J=6.35 Hz, 3H), 0.90 (s, 3H), 0.67 (s, 3H); ¹³C NMR (CDCl₃, 75 MHz) δ 119.91, 119.89, 80.75, 79.65, 76.29, 65.83, 65.37, 65.19, 63.63, 46.57, 46.44, 42.77, 41.79, 39.71, 35.63, 35.26, 35.02, 32.00, 29.46, 29.03, 27.96, 27.74, 26.64, 26.42, 26.12, 23.56, 22.98, 22.95, 18.24, 14.65, 14.54, 14.30, 12.60; HRFAB-MS (thioglycerol+Na⁺ matrix) m/e: ([M+Na]⁺) 618.4247 (67.8%), calcd. 618.4247.

Compound 29: To a solution of 28 (0.34 g, 0.57 mmol) in dry CH₂ Cl₂ (15 mL) under N₂ at 0° C. was added NEt₃ (119.5 μL, 0.857 mmol) followed by the addition of mesyl chloride (53.1.mu.L, 0.686 mmol). The mixture was allowed to stir at 0° C. for 30 minutes before the addition of H₂O (6 mL). After the introduction of brine (60 mL), the aqueous mixture was extracted with EtOAc (4×20 mL). The combined extracts were washed with brine once, dried over anhydrous Na₂ SO₄ and concentrated. To the residue was added N-benzylmethyl amine (0.5 mL) and the mixture was stirred under N₂ at 80° C. overnight. Excess N-benzylmethylamine was removed in vacuo and the residue was subject to column chromatography (silica gel, EtOAc/hexanes 2:1 followed by EtOAc) to afford product (0.35 g, 88% yield) as a pale yellow oil. IR (neat) 2940, 2863, 2785, 2249, 1469, 1453, 1366, 1348, 1108 cm¹; ¹H NMR (CDCl₃, 300 MHz) δ 7.34−7.21 (m, 5H), 3.76−3.69 (m, 1H), 3.64−3.50 (m, 4H), 3.48 (s, 2H), 3.31−3.05 (series of multiplets, 4H), 2.52−2.46 (m, 6H), 2.33 (t, J=7.32H, 2 Hz), 2.18 (s, 3H), 2.13−0.95 (series of multiplets, 30H), 0.91 (d, J=6.80H, 3 Hz), 0.90 (s, 3H), 0.66 (s, 3H); ¹³C NMR (CDCl₃, 75 MHz) δ 139.37, 129.17, 128.26, 126.93, 119.96, 119.91, 80.73, 79.59, 76.26, 65.79, 65.35, 65.13, 62.47, 58.25, 46.74, 46.40, 42.72, 42.38, 41.76, 39.68, 35.78, 35.22, 34.98, 33.79, 28.99, 27.92, 27.71, 26.63, 26.38, 26.09, 24.21, 23.54, 22.96, 22.90, 18.28, 14.62, 14.51, 14.26, 12.58; HRFAB-MS (thioglycerol+Na⁺ matrix) m/e: ([M+H]⁺) 699.5226 (100%), calcd. 699.5213.

Compound 3: A solution of 29 (0.074 g, 0.106 mmol) in anhydrous THF (10 mL) was added dropwise to a mixture of AlCl₃ (0.1414 g, 1.06 mmol) and LiAlH₄ (0.041 g, 1.06 mmol) in dry THF (10 mL). The suspension was stirred for 24 hours followed by the addition of 20% NaOH aqueous solution (2 mL) at ice-bath temperature. Anhydrous Na₂ SO₄ was added to the aqueous slurry. The solution was filtered and the precipitate washed twice with THF. After removal of solvent, the residue was subject to column chromatography (silica gel, MeOH/CH₂ Cl₂ 1:1 followed by MeOH/CH₂ Cl₂/NH₃.H₂O 4:4:1) to afford the desired product (0.038 g, 50% yield) as a clear oil. IR (neat) 3362, 2935, 2863, 2782, 1651, 1574, 1568, 1557, 1471, 1455, 1103 cm⁻¹; ¹H NMR (CDCl₃, 300 MHz) δ 7.32−7.22 (m, 5H), 3.60−3.02 (series of broad multiplets, 18H), 2.90−2.70 (m, 5H), 2.33 (t, J=7.20 Hz, 2H), 2.24−2.04 (m, 3H), 2.18 (s, 3H), 1.96−0.96 (series of multiplets, 30H), 0.90 (d, J=7.57 Hz, 3H), 0.89 (s, 3H), 0.64 (s, 3H); ¹³C NMR (CDCl₃, 75 MHz) δ 139.44, 129.24, 128.31, 126.97, 80.63, 79.65, 75.97, 68.44, 68.00, 67.96, 62.54, 58.40, 46.77, 46.30, 42.73, 42.43, 42.07, 41.92, 41.74, 41.72, 39.81, 35.82, 35.48, 35.07, 33.84, 31.04, 30.30, 30.10, 29.03, 28.11, 27.82, 27.81, 27.74, 27.67, 27.64, 24.31, 23.50, 23.04, 22.93, 18.22, 12.63; HRFAB-MS (thioglycerol+Na⁺ matrix) m/e: ([M+H]⁺) 711.6139 (100%), calcd. 711.6152; ([M+Na]⁺) 733.5974 (46.1%), calcd. 733.5972.

### Example 3

This example includes a description of one or more exemplary synthestic procedures for obtaining Compounds 6, 7 and 30−33.

Compound 30: Cholic acid (3.0 g, 7.3 mmol) was dissolved in CH₂Cl₂ (50 mL) and methanol (5 mL). Dicyclohexylcarbodiimide (DCC) (1.8 g, 8.8 mmol) was added followed by N-hydroxysuccinimide (about 100 mg) and benzylmethylamine (1.1 g, 8.8 mmol). The mixture was stirred for 2 hours, then filtered. The filtrate was concentrated and chromatographed (SiO₂, 3% MeOH in CH₂Cl₂) to give 3.0 g of a white solid (81% yield). m.p. 184-186° C.; IR (neat) 3325, 2984, 1678 cm⁻¹; ¹H NMR (CDCl₃, 200 MHz) δ 7.21 (m, 5H), 4.51 (m, 2H), 3.87 (m, 1H), 3.74 (m, 2H), 3.36 (m, 2H), 2.84 (s, 3H), 2.48−0.92 (series of multiplets, 28H), 0.80 (s, 3H), 0.58 (d, J=6.5 Hz, 3H); ¹³C NMR (CDCl₃, 50 MHz) δ 174.30, 173.94, 137.36, 136.63, 128.81, 128.46, 127.85, 127.50, 127.18, 126.28, 72.96, 71.76, 68.35, 53.39, 50.65, 48.77, 46.91, 46.33, 41.44, 39.36, 39.18, 35.76, 35.27, 34.76, 33.87, 31.54, 34.19, 31.07, 30.45, 28.11, 27.63, 26.14, 25.59, 24.92, 23.26, 17.51, 12.41; FAB-MS (thioglycerol+Na⁺ matrix) m/e: ([M+H]⁺) 512 (100%), calcd. 512.

Compound 31:Compound 30 (2.4 g, 4.7 mmol) was added to a suspension of LiAlH₄ (0.18 g, 4.7 mmol) in THF (50 mL). The mixture was refluxed for 24 hours, then cooled to 0° C. An aqueous solution of Na₂SO₄ was carefully added until the grey color of the mixture dissipated. The salts were filtered out, and the filtrate was concentrated in vacuo to yield 2.1 g of a white solid (88%). The product proved to be of sufficient purity for further reactions. m.p. 70-73° C.; IR (neat) 3380, 2983, 1502 cm⁻¹; ¹H NMR (CDCl₃, 300 MHz) δ 7.23 (m, 5H), 3.98 (bs, 2H), 3.81 (m, 3H), 3.43 (m, 3H), 2.74 (m, 2H), 2.33 (m, 3H), 2.25 (s, 3H), 2.10−0.90 (series of multiplets, 24H), 0.98 (s, 3H), 0.78 (s, 3H); ¹³C NMR (CDCl₃, 75 MHz) δ 135.72, 129.63, 128.21, 128.13, 125.28, 72.91, 71.63, 62.05, 60.80, 56.79, 47.00, 46.23, 41.44, 40.81, 39.41, 35.42, 35.24, 34.63, 34.02, 33.22, 31.73, 30.17, 29.33, 29.16, 28.02, 27.49, 26.17, 25.55, 23.10, 22.48, 22.33, 17.54, 12.65; FAB-MS (thioglycerol matrix) m/e: ([M+H]⁺) 498 (100%), calcd. 498.

Compound 32: Compound 31 (0.36 g, 0.72 mmol) was dissolved in CH₂Cl₂ (15 mL) and Bocglycine (0.51 g, 2.89 mmol), DCC (0.67 g, 3.24 mmol) and dimethylaminopyridine (DMAP) (about 100 mg) were added. The mixture was stirred under N₂ for 4 hours then filtered. After concentration and chromatography (SiO₂, 5% MeOH in CH₂Cl₂), the product was obtained as a 0.47 g of a clear glass (68%). ¹H NMR (CDCl₃, 300 MHz) δ 7.30 (m, 5H), 5.19 (bs, 1H), 5.09 (bs, 3H), 5.01 (bs, 1H), 4.75 (m, 1H), 4.06−3.89 (m, 6H), 2.33 (m, 2H), 2.19 (s, 3H) 2.05−1.01 (series of multiplets, 26H), 1.47 (s, 9H), 1.45 (s, 18H), 0.92 (s, 3H), 0.80 (d, J=6.4 Hz, 3H), 0.72 (s, 3H). ¹³C NMR (CDCl₃, 75 MHz) δ 170.01, 169.86, 169.69, 155.72, 155.55, 139.90, 129.05, 128.17, 126.88, 79.86, 76.53, 75.09, 72.09, 62, 35, 57.88, 47.78, 45.23, 43.12, 42.79, 42.16, 40.81, 37.94, 35.51, 34.69, 34.57, 34.36, 33.30, 31.31, 29.66, 28.80, 28.34, 27.22, 26.76, 25.61, 24.02, 22.83, 22.47, 17.93, 12.19; FAB-MS (thioglycerol matrix) m/e: ([M+H]⁺) 970 (100%), calcd. 970.

Compound 33: Compound 31 (0.39 g, 0.79 mmol) was dissolved in CH₂Cl₂ (15 mL) and Boc-β-alanine (0.60 g, 3.17 mmol), DCC (0.73 g, 3.56 mmol) and dimethylaminopyridine (DMAP) (about 100 mg) were added. The mixture was stirred under N₂ for 6 hours then filtered. After concentration and chromatography (SiO₂, 5% MeOH in CH₂Cl₂), the product was obtained as a 0.58 g of a clear glass (72%). IR (neat) 3400, 2980, 1705, 1510 cm⁻¹; ¹H NMR (CDCl₃, 300 MHz) δ 7.27 (m, 5H), 5.12 (bs, 4H), 4.93 (bs, 1H), 4.71 (m, 1H), 3.40 (m, 12H), 2.59−2.48 (m, 6H), 2.28 (m, 2H), 2.17 (s, 3H) 2.05−1.01 (series of multiplets, 26H), 1.40 (s, 27H), 0.90 (s, 3H), 0.77 (d, J=6.1 Hz, 3H), 0.70 (s, 3H). ¹³C NMR (CDCl₃, 75 MHz) δ 171.85, 171.50, 171.44, 155.73, 138.62, 129.02, 128.09, 126.87, 79.18, 75.53, 74.00, 70.91, 62.20, 57.67, 47.84, 44.99, 43.28, 41.98, 40.73, 37.67, 36.12, 34.94, 34.65, 34.47, 34.20, 33.29, 31.23, 29.57, 28.74, 28.31, 28.02, 27.86, 27.12, 26.73, 25.46, 24.86, 23.95, 22.77, 22.39, 17.91, 12.14; HRFAB-MS (thioglycerol+Na⁺ matrix) m/e: ([M+H]⁺) 1011.6619 (100%), calcd. 1011.6634.

Compound 6: Compound 32 (0.15 g, 0.15 mmol) was stirred with excess 4 N HCl in dioxane for 40 minutes. The dioxane and HCl were removed in vacuo leaving 0.12 g of a clear glass (about 100%). ¹H NMR (CD₃OD, 300 MHz) δ 7.62 (bs, 2H), 7.48 (bs, 3H), 5.30 (bs, 1H), 5.11 (bs, 1H), 4.72 (bs (1H), 4.46 (m, 1H), 4.32 (m, 1H) 4.05−3.91 (m, 4H), 3.10 (m, 2H), 2.81 (s, 3H), 2.15−1.13 (series of multiplets, 25H), 1.00 (s, 3H), 0.91 (bs, 3H), 0.82 (s, 3H). ¹³C NMR (CD₃OD, 125 MHz) δ 166.86, 166.50, 131.09, 130.18, 129.17, 128.55, 76.60, 75.43, 72.61, 72.04, 70.40, 66.22, 60.07, 58.00, 57.90, 54.89, 54.76, 46.44, 44.64, 43.39, 42.22, 38.56, 36.78, 34.14, 33.92, 33.84, 31.82, 30.54, 29.67, 28.79, 27.96, 26.79, 26.00, 24.99, 23.14, 22.05, 21.82, 19.91, 17.27, 11.60; HRFAB-MS (thioglycerol+Na⁺ matrix) m/e: ([M-4 Cl-3 H]⁺) 669.4576 (100%), calcd. 669.4591.

Compound 7: Compound 33 (0.20 g, 0.20 mmol) was stirred with excess 4 N HCl in dioxane for 40 minutes. The dioxane and HCl were removed in vacuo leaving 0.12 g of a clear glass (about 100%). ¹H NMR (CD₃OD, 500 MHz) δ 7.58 (bs, 2H), 7.49 (bs, 3H), 5.21 (bs, 1H), 5.02 (bs, 1H), 4.64 (m, 1H), 4.44 (m, 1H), 4.28 (m, 1H), 3.30−2.84 (m, 14H), 2.80 (s, 3H), 2.11−1.09 (series of multiplets, 25H), 0.99 (s, 3H), 0.89 (d, J=4.1 Hz, 3H), 0.80 (s, 3H); ¹³C NMR (CD₃ OD, 125 MHz) δ 171.92, 171.56, 171.49, 132.44, 131.32, 131.02, 130.51, 78.13, 76.61, 61.45, 57.94, 46.67, 44.80, 42.36, 40.85, 39.33, 37.03, 36.89, 36.12, 36.09, 35.79, 35.63, 33.81, 33.10, 32.92, 32.43, 30.28, 28.43, 28.04, 26.65, 24.02, 22.86, 21.98, 18.70, 12.68; HRFAB-MS (thioglycerol+Na⁺ matrix) m/e: ([M-4 Cl-3H]⁺) 711.5069 (43%), calcd. 711.5061.

### Example 4

This example includes a description of one or more exemplary synthestic procedures for obtaining Compounds 8, CSA-7, CSA-8 and 34−40.

Compound 34: Diisopropyl azodicarboxylate (DIAD) (1.20 mL, 6.08 mmol) was added to triphenylphosphine (1.60 g, 6.08 mmol) in THF (100 mL) at 0° C. and was stirred for half an hour during which time the yellow solution became a paste. Compound 14 (2.58 g, 4.06 mmol) and p-nitrobenzoic acid (0.81 g, 4.87 mmol) were dissolved in THF (50 mL) and added to the paste. The resulted mixture was stirred at ambient temperature overnight. Water (100 mL) was added and the mixture was made slightly basic by adding NaHCO₃ solution followed by extraction with EtOAc (3×50 mL). The combined extracts were washed with brine once and dried over anhydrous Na₂ SO₄. The desired product (2.72 g, 85% yield) was obtained as white powder after SiO₂ chromatography (Et₂ O/hexanes 1:2). m.p. 207-209° C.; IR (KBr) 3434, 3056, 2940, 2868, 1722, 1608, 1529, 1489, 1448, 1345 cm⁻¹; ¹H NMR (CDCl₃, 300 MHz) δ 8.30−8.26 (m, 2H), 8.21−8.16 (m, 2H), 7.46−7.42 (m, 6H), 7.31−7.18 (m, 9H) 5.33 (bs, 1H), 4.02 (bs, 1H), 3.90 (bs, 1H), 3.09−2.97 (m, 2H), 2.68 (td, J=14.95, 2.56 Hz, 1H), 2.29−2.19 (m, 1H), 2.07−1.06 (series of multiplets, 24H), 1.01 (s, 3H), 0.98 (d, J=6.6 Hz, 3H), 0.70 (s, 3H); ¹³C NMR (CDCl₃, 75 MHz) δ 164.21, 150.56, 144.70, 136.79, 130.77, 128.88, 127.86, 126.98, 123.70, 86.47, 73.24, 73.00, 68.70, 64.22, 47.79, 46.79, 42.15, 39.76, 37.47, 35.52, 35.34, 34.23, 33.79, 32.46, 31.12, 28.74, 27.71, 26.85, 26.30, 25.16, 23.41, 17.98, 12.77; HRFAB-MS (thioglycerol+Na⁺ matrix) m/e: ([M+Na]⁺) 808.4203 (53.8%), calcd. 808.4189. Nitrobenzoate (2.75 g, 3.5 mmol) was dissolved in CH₂Cl₂ (40 mL) and MeOH (20 mL) and 20% aqueous NaOH (5 mL) were added. The mixture was heated up to 60° C. for 24 hours. Water (100 mL) was introduced and extracted with EtOAc. The combined extracts were washed with brine and dried over anhydrous Na₂ SO₄. The desired product (1.89 g, 85% yield) was obtained as white solid after SiO₂ chromatography (3% MeOH in CH₂ Cl₂ as eluent). m.p. 105-106° C.; IR (KBr) 3429, 3057, 2936, 1596, 1489, 1447, 1376, 1265, 1034, 704 cm⁻¹; ¹H NMR (CDCl₃, 300 MHz) δ 7.46−7.42 (m, 6H), 7.32−7.19 (m, 9H), 4.06 (bs, 1H), 3.99 (bs, 1H), 3.86 (bd, J=2.44 Hz, 1H), 3.09−2.97 (m, 2H), 2.47 (td, J=14.03, 2.44 Hz, 1H), 2.20−2.1 (m, 1H), 2.04−1.04 (series of multiplets, 25H), 0.97 (d, J=6.59 Hz, 3H), 0.94 (s, 3H), 0.68 (s, 3H); ¹³C NMR (CDCl₃, 75 MHz) δ 144.70, 128.88, 127.86, 126.97, 86.45, 73.31, 68.84, 67.10, 64.23, 47.71, 46.74, 42.10, 39.70, 36.73, 36.73, 36.15, 35.53, 35.45, 34.45, 32.46, 29.93, 28.67, 27.86, 27.71, 26.87, 26.04, 23.43, 23.16, 17.94, 12.75; HRFAB-MS (thioglycerol+Na⁺ matrix) m/e: ([M+Na]⁺) 659.4064 (100%), calcd. 659.4076.

Compound 35: To a round-bottom flask were added 34 (2.0 g, 3.14 mmol), NaH (60% in mineral oil, 3.8 g, 31.4 mmol) and THF (150 mL). The suspension was refluxed for 2 hours followed by the addition of allyl bromide (2.72 mL, 31.4 mL). After refluxing for 28 hours, another 10 eq. of NaH and allyl bromide were added. After 72 hours, another 10 eq. of NaH and allyl bromide were added. After 115 hours, TLC showed almost no starting material or intermediates. Water (100 mL) was added to the suspension carefully, followed by extraction with EtOAc (5×50 mL). The combined extracts were washed with brine and dried over anhydrous Na₂SO₄. The desired product (1.81 g, 79% yield) was obtained as a yellowish glass after SiO₂ chromatography (5% EtOAc/hexanes). IR (neat) 3060, 3020, 2938, 2865, 1645, 1596, 1490, 1448, 1376, 1076, 705 cm⁻¹; ¹H NMR (CDCl₃, 300 MHz) δ 7.46−7.42 (m, 6H), 7.31−7.18 (m, 9H), 6.06−5.85 (m, 3H), 5.35−5.20 (m, 3H), 5.15−5.06 (m, 3H), 4.10−4.00 (m, 2H), 3.93−3.90 (m, 2H), 3.85−3.79 (ddt, J=13.01, 4.88, 1.59 Hz, 1H), 3.73−3.66 (ddt, J=13.01, 5.38, 1.46 Hz, 1H), 3.58 (bs, 1H), 3.54 (bs, 1H), 3.32 (d, J=2.93 Hz, 1H), 3.07−2.96 (m, 2H), 2.36 (td, J=13.67, 2.68 Hz, 1 H), 2.24−2.10 (m, 2H), 2.03−1.94 (m, 1H), 1.87−0.86 (series of multiplets, 20H), 0.91 (s, 3H), 0.90 (d, J=6.83 Hz, 3H), 0.64 (s, 3H); ¹³C NMR (CDCl₃, 75 MHz) δ 144.77, 136.29, 136.21, 136.13, 128.90, 127.86, 126.94, 116.13, 115.51, 115.42, 86.44, 81.11, 75.65, 73.92, 69.40, 68.81, 64.43, 46.68, 46.54, 42.93, 39.93, 36.98, 35.66, 35.14, 35.14, 32.83, 32.54, 30.48, 28.51, 27.72, 27.64, 26.82, 24.79, 23.65, 23.43, 23.40, 18.07, 12.80; HRFAB-MS (thioglycerol+Na⁺ matrix) m/e: ([M+H]⁺) 757.5185 (12.9%), calcd. 757.5196.

Compound 36: Ozone was bubbled through a solution of 35 (0.551 g, 0.729 mmol) in CH₂ Cl₂ (40 mL) and MeOH (20 mL) at −78° C. until the solution turned a deep blue. Excess ozone was blown off with oxygen. Methylsulfide (1 mL) was added followed by the addition of NaBH₄ (0.22 g, 5.80 mmol) in 5% NaOH solution and methanol. The resulted mixture was stirred overnight at room temperature and washed with brine. The brine was then extracted with EtOAc (3×20 mL). The combined extracts were dried over Na₂ SO₄. The desired product (0.36 g, 65% yield) was obtained as a colorless glass after SiO₂ chromatography (5% MeOH/CH₂ Cl₂). IR (neat) 3396, 3056, 2927, 1596, 1492, 1462, 1448, 1379, 1347, 1264, 1071 cm⁻¹; ¹H NMR (CDCl₃, 300 MHz) δ 7.46−7.42 (m, 6H), 7.32−7.18 (m, 9H), 3.77−3.57 (series of multiplets, 10H), 3.48−3.44 (m, 2H), 3.36−3.30 (m, 2H), 3.26−3.20 (m, 1H), 3.04−2.99 (m, 2H), 2.37−0.95 (series of multiplets, 27H), 0.92 (s, 3H), 0.91 (d, J=6.59 Hz, 3H), 0.67 (s, 3H); ¹³C NMR (CDCl₃, 75 MHz) δ 144.69, 128.87, 127.84, 126.94, 86.44, 81.05, 76.86, 74.65, 69.91, 69.22, 68.77, 64.24, 62.44, 62.42, 62.26, 46.92, 46.54, 42.87, 39.73, 36.86, 35.52, 35.13, 32.82, 32.54, 30.36, 28.71, 27.61, 27.44, 26.79, 24.82, 23.51, 23.38, 23.31, 18.28, 12.74; HRFAB-MS (thioglycerol+Na⁺ matrix) m/e: ([M+Na]⁺) 791.4844 (96.4%), calcd. 791.4863.

Compound 37: NEt₃ (0.23 mL, 1.66 mmol) was added to a solution of 36 (0.364 g, 0.47 mmol) in dry CH₂ Cl₂ (30 mL) at 0° C. under N₂ followed by the introduction of mesyl chloride (0.12 mL, 1.56 mmol). The mixture was stirred for 10 minutes and H₂ O (10 mL) added to quench the reaction, followed by extraction with EtOAc (3×30 mL). The combined extracts were washed with brine and dried over anhydrous Na₂ SO₄. SiO₂ chromatography (EtOAc/hexanes 1:1) gave the desired product (0.411 g, 86% yield) as white glass. IR (neat) 3058, 3029, 2939, 2868, 1491, 1461, 1448, 1349, 1175, 1109, 1019 cm⁻¹; ¹H NMR (CDCl₃, 300 MHz) δ 7.46−7.42 (m, 6H), 7.31−7.19 (m, 9H), 4.35−4.26 (m, 6H), 3.84−3.74 (m, 2H), 3.64−3.56 (m, 4H), 3.49−3.34 (m, 3H), 3.06 (s, 3H), 3.04 (s, 3H), 3.02 (s, 3H), 3.09−2.95 (m, 2H), 2.28 (bt, J=14.89 Hz, 1H), 2.09−0.86 (series of multiplets, 21H), 0.92 (s, 3H), 0.90 (d, J=6.78 Hz, 3H), 0.66 (s, 3H); ¹³C NMR (CDCl₃, 75 MHz) δ 144.66, 128.86, 127.86, 126.97, 86.46, 81.28, 77.18, 75.00, 70.14, 69.89, 69.13, 66.49, 65.85, 65.72, 64.22, 47.06, 46.35, 42.77, 39.58, 37.81, 37.64, 37.55, 36.75, 35.48, 35.02, 32.59, 32.52, 30.27, 28.43, 27.56, 27.52, 26.92, 24.62, 23.34, 23.25, 23.10, 18.24, 12.64; HRFAB-MS (thioglycerol+Na⁺ matrix) m/e: ([M+Na]⁺) 1025.4207 (100%), calcd. 1025.4189.

Compound 38: The suspension of 37 (0.227 g, 0.227 mmol) and NaN₃ (0.147 g, 2.27 mmol) in dry DMSO (5 mL) was stirred at 80° C. overnight, diluted with H₂O (50 mL) and extracted with EtOAc (3×20 mL). The extracts were washed with brine once and dried over anhydrous Na₂ SO₄. SiO₂ chromatography (EtOAc/hexanes 1:8) afforded the desired product (0.153 g, 80% yield) as a yellow oil. IR (neat) 2929, 2866, 2105, 1490, 1466, 1448, 1107, 705 cm⁻¹; ¹H NMR (CDCl₃, 300 MHz) δ 7.46−7.42 (m, 6H), 7.32−7.19 (m, 9H), 3.80−3.74 (m, 1H), 3.70−3.55 (series of multiplets, 5H), 3.41−3.19 (series of multiplets, 9H), 3.04−2.98 (m, 2H), 2.41 (td, J=13.1, 2.44 Hz, 1H), 2.29−2.14 (m, 2H), 2.04−0.86 (series of multiplets, 20H), 0.93 (s, 3H), 0.91 (d, J=6.60 Hz, 3H), 0.66 (s, 3H); ¹³C NMR (CDCl₃, 75 MHz) δ 144.78, 128.93, 127.87, 126.96, 86.46, 81.30, 77.16, 75.21, 67.99, 67.44, 67.03, 64.41, 51.64, 51.57, 51, 33, 46.71, 46.30, 42.35, 39.75, 36.72, 35.64, 35.20, 32.52, 32.42, 30.17, 28.63, 27.80, 27.22, 26.90, 24.80, 23.55, 23.30, 23.24, 18.23, 12.65; HRFAB-MS (thioglycerol+Na⁺ matrix) m/e: ([M+Na]⁺) 866.5049 (96.9%), calcd. 866.5057.

Compound 39: p-Toluenesulfonic acid (1.72 mg) was added into the solution of 38 (0.153 g, 0.18 mmol) in CH₂ Cl₂ (5 mL) and MeOH (5 mL), and the mixture was stirred for 2.5 hours. Saturated NaHCO₃ solution (5 mL) was introduced followed by the introduction of brine (30 mL). The aqueous mixture was extracted with EtOAc and the combined extracts washed with brine and dried over Na₂ SO₄. The desired product (0.10 g, 92% yield) was obtained as a pale yellowish oil after SiO₂ chromatography (EtOAc/hexanes 1:3). IR (neat) 3426, 2926, 2104, 1467, 1441, 1347, 1107 cm¹; ¹H NMR (CDCl₃, 300 MHz) δ 3.81−3.74 (m, 1H), 3.71−3.54 (m, 7H), 3.41−3.19 (m, 9H), 2.41 (td, J=13.61, 2.32 Hz, 1H), 2.30−2.14 (m, 2H), 2.07−1.98 (m, 1H), 1.94−0.95 (series of multiplets, 21H), 0.95 (d, J=6.35 Hz, 3H), 0.93 (s, 3H), 0.69 (s, 3H); ¹³C NMR (CDCl₃, 75 MHz) δ 81.22, 77.08, 75.13, 67.94, 67.36, 66.97, 63.76, 51.59, 51.51, 51.26, 46.51, 46.24, 42.31, 39.68, 36.64, 35.58, 35.12, 32.34, 31.92, 30.11, 29.55, 28.54, 27.82, 27.16, 24.75, 23.47, 23.23, 23.18, 18.15, 12.56; HRFAB-MS (thioglycerol+Na⁺ matrix) m/e: ([M+Na]⁺) 624.3966 (54.9%), calcd. 624.3962.

Compound 40: To a solution of 39 (0.10 g, 0.166 mmol) in CH₂Cl₂ (8 mL) at 0° C. was added NEt₃ (34.8 μL, 0.25 mmol) under N₂ followed by the introduction of mesyl chloride (15.5.mu.L, 0.199 mmol). The mixture was stirred 15 minutes. Addition of H₂ O (3 mL) and brine (20 mL) was followed by extraction with EtOAc (4×10 mL). The combined extracts were washed with brine once and dried over Na₂ SO₄. After removal of solvent, the residue was mixed with N-benzylmethylamine (0.5 mL) and heated to 80° C. under N₂ overnight. Excess N-benzyl methylamine was removed in vacuo and the residue was subjected to SiO₂ chromatography (EtOAc/hexanes 1:4) to give the product (0.109 g, 93% yield) as a yellow oil. IR (neat) 2936, 2784, 2103, 1467, 1442, 1346, 1302, 1106, 1027 cm⁻¹; ¹H NMR (CDCl₃, 300 MHz) δ 7.32−7.23 (m, 5H), 3.81−3.74 (m, 1H), 3.71−3.55 (m, 5H), 3.47 (s, 2H), 3.41−3.19 (m, 9H), 2.46−2.11 (m, 5H), 2.18 (s, 3H), 2.03−0.85 (series of multiplets, 20H), 0.93 (s, 3H), 0.93 (d, J=6.35 Hz, 3H), 0.67 (s, 3 H); ¹³C NMR(CDCl₃, 75 MHz) δ 139.54, 129.26, 128.32, 126.97, 81.26, 77.12, 75.17, 67.98, 67.42, 67.00, 62.50, 58.41, 51.61, 51.54, 51.29, 46.66, 46.28, 42.46, 42.32, 39.72, 36.68, 35.76, 35.16, 33.75, 32.38, 30.15, 28.59, 27.85, 27.19, 24.77, 24.15, 23.53, 23.28, 23.22, 18.28, 12.60; HRFAB-MS (thioglycerol+Na⁺ matrix) m/e: ([M+H]⁺) 705.4929 (100%), calcd. 705.4928.

Compound 8: A suspension of 40 (0.109 g, 0.155 mmol) and LiAlH₄ (23.5 mg, 0.62 mmol) in THF (20 mL) was stirred under N₂ overnight. Na₂ SO₄. 10H₂ O was carefully added and stirred until no grey color persisted. Anhydrous Na₂SO₄ was added and the white precipitate was filtered out and rinsed with dry THF. After removal of solvent, the residue was dissolved in minimum CH₂Cl₂ and filtered. The desired product (0.091 g, 94% yield) was obtained as a colorless oil after the solvent was removed. IR (neat) 3371, 3290, 3027, 2938, 2862, 2785, 1586, 1493, 1453, 1377, 1347, 1098 cm⁻¹; ¹H NMR (CDCl₃, 300 MHz) δ 7.31−7.21 (m, 5H), 3.65−3.53 (m, 4H), 3.47 (s, 2H), 3.42−3.34 (m, 2H), 3.30 (bs, 1H), 3.26−3.20 (m, 1H), 3.14−3.09 (m, 1H), 2.89−2.81 (m, 6H), 2.39−2.27 (m, 3H), 2.17 (s, 3H), 2.15−0.88 (series of multiplets, 29H), 0.93 (d, J=6.59 Hz, 3H), 0.92 (s, 3H), 0.67 (s, 3H); ¹³C NMR (CDCl₃, 75 MHz) δ 139.34, 129.16, 28.24, 126.90, 80.75, 76.44, 74.29, 70.58, 69.88, 69.75, 62.47, 58.27, 46.66, 46.47, 42.75, 42.63, 42.51, 42.35, 39.77, 36.87, 35.73, 35.04, 33.77, 32.90, 30.38, 28.71, 27.70, 27.32, 24.89, 24.09, 23.53, 23.36, 23.25, 18.24, 12.62; HRFAB-MS (thioglycerol+Na⁺ matrix) m/e: ([M+H]⁺) 627.5199 (23.3%), calcd. 627.5213.

Compound CSA-7: To a solution of 23 (0.18 g, 0.28 mmol) in dry DMF (4 mL) were added NaH (0.224 g, 60% in mineral oil, 5.60 mmol) and 1-bromo octane (0.48 mL, 2.80 mmol). The suspension was stirred under N₂ at 65° C. overnight followed by the introduction of H₂ O (60 mL) and extraction with ether (4×20 mL). The combined extracts were washed with brine and dried over Na₂ SO₄. SiO₂ chromatography (hexanes and 5% EtOAc in hexanes) afforded the desired product (0.169 g, 80% yield) as a pale yellowish oil. IR (neat) 2927, 2865, 2099, 1478, 1462, 1451, 1350, 1264, 1105 cm¹; ¹H NMR (CDCl₃, 300 MHz) δ 3.69−3.35 (series of multiplets, 15H), 3.26−3.02 (series of multiplets, 4H), 2.19−2.02 (m, 3H), 1.97−1.16 (series of multiplets, 37H), 1.12−0.99 (m, 2H), 0.92−0.86 (m, 9H), 0.65 (s, 3H); ¹³C NMR (CDCl₃, 75 MHz) δ 80.69, 79.84, 76.13, 71.57, 71.15, 65.07, 64.49, 64.39, 49.08, 48.99, 48.80, 46.68, 46.45, 42.72, 42.05, 39.88, 35.74, 35.49, 35.36, 35.14, 32.42, 32.03, 30.01, 29.85, 29.81, 29.76, 29.67, 29.48, 29.14, 27.92, 27.80, 27.70, 26.58, 26.42, 23.59, 23.09, 22.92, 22.86, 18.11, 14.31, 12.65; HRFAB-MS (thioglycerol+Na⁺ matrix) m/e: ([M+Na]⁺) 778.5685 (22.1%), calcd. 778.5683. The triazide (0.169 g, 0.224 mmol) and LiAlH₄ (0.025 g, 0.67 mmol) were suspended in anhydrous THF (10 mL) and stirred under N₂ at room temperature overnight followed by careful introduction of Na₂ SO₄ hydrate. After the grey color disappeared, anhydrous Na₂ SO₄ was added and stirred. The white precipitate was removed by filtration and washed with THF. After removal of solvent, the residue was dissolved in 1 M hydrochloric acid and the aqueous solution was extracted with ether (5 mL) once. The aqueous solution was then made basic by adding 20% aqueous NaOH solution followed by extraction with Et₂ O (4×5 mL). The combined extracts were washed, dried and concentrated. The residue was then subject to SiO₂ chromatography (MeOH/CH₂Cl₂ (1:1) followed by MeOH/CH₂Cl₂/NH₃. H₂ O (4:4:1)) to afford the desired product (0.091 g, 60% yield) as a colorless oil. IR (neat) 3361, 2927, 2855, 1576, 1465, 1351, 1105 cm⁻¹; ¹H NMR (CD₃OD, 300 MHz) δ 4.86 (bs, 6H), 3.77−3.72 (m, 1H), 3.70−3.61 (m, 1H), 3.57−3.53 (m, 3H), 3.43−3.38 (m, 4H), 3.34−3.27 (m, 2H), 3.18−3.10 (m, 2H), 2.84−2.71 (m, 6H), 2.22−2.07 (m, 3H), 2.00−1.02 (series of multiplets, 39H), 0.97−0.88 (m, 9H), 0.71 (s, 3H); ¹³C NMR (CD₃ OD, 75 MHz) δ 82.20, 81.00, 77.62, 72.52, 72.06, 68.00, 67.92, 67.39, 48.20, 47.53, 44.26, 43.40, 41.42, 41.15, 40.84, 40.35, 36.88, 36.73, 36.42, 36.11, 34.24, 34.05, 33.94, 33.67, 33.17, 30.95, 30.72, 30.62, 29.81, 29.35, 28.87, 28.79, 27.51, 24.57, 23.90, 23.83, 23.44, 18.76, 14.62, 13.07; HRFAB-MS (thioglycerol matrix) m/e: ([M+H]⁺) 678.6133 (100%), calcd. 678.6149.

Compound CSA-8: A suspension of 23 (0.126 g, 0.196 mmol) and LiAlH₄ (0.037 g, 0.98 mmol) in THF (40 mL) was stirred at room temperature under N₂ overnight followed by careful addition of Na₂SO₄. 10H₂O. After the grey color in the suspension disappeared, anhydrous Na₂SO₄ was added and stirred until organic layer became clear. The white precipitate was removed by filtration and washed with twice THF. The THF was removed in vacuo, and the residue was subject to SiO₂ chromatography (MeOH/CH₂Cl₂/NH₃/H₂O (4:4:1)) to afford the desired product (0.066 g, 60% yield) as a colorless oil. IR (neat) 3365, 2933, 2865, 1651, 1471, 1455, 1339, 1103 cm⁻¹; ¹H NMR (CDCl₃/30% CD₃OD, 300 MHz) δ 4.43 (bs, 7H), 3.74−3.68 (m, 1H), 3.66−3.60 (m, 1H), 3.57−3.50 (m, 5H), 3.34−3.25 (M, 2H), 3.17−3.06 (M, 2H), 2.84−2.74 (M, 6H), 2.19−2.01 (M, 3H), 1.97−0.96 (series of multiplets, 27H), 0.94 (d, J=7.2 Hz, 3H), 0.92 (s, 3H), 0.69 (s, 3H); ¹³C NMR (CDCl₃, 75 MHz) δ 80.44, 79.27, 75.77, 66.59, 66.53, 65.86, 62.51, 46.21, 45.84, 42.55, 41.53, 40.09, 39.43, 39.31, 39.02, 35.16, 34.93, 34.86, 34.57, 32.93, 32.71, 31.57, 28.66, 28.33, 27.64, 27.22, 23.04, 22.40, 22.29, 17.60, 11.98; HRFAB-MS (thioglycerol+Na⁺ matrix) m/e: ([M+H]⁺) 566.4889 (8.9%), calcd. 566.4897.

### Example 5

This example includes a description of one or more exemplary synthestic procedures for obtaining Compounds CSA-11 and 43-47.

Compound 43: Precursor compound 41 was prepared following the method reported by D. H. R. Barton, J. Wozniak, S. Z. Zard, Tetrahedron, 1989, vol. 45, 3741−3754. A mixture of 41 (1.00 g, 2.10 mmol), ethylene glycol (3.52 mL, 63 mmol) and p-TsOH (20 mg, 0.105 mmol) was refluxed in benzene under N₂ for 16 hours. Water formed during the reaction was removed by a Dean-Stark moisture trap. The cooled mixture was washed with NaHCO₃ solution (50 mL) and extracted with Et₂O (50 mL, 2×30 mL). The combined extracts were washed with brine and dried over anhydrous Na₂ SO₄. Removal of the solvent gave the product (1.09 g, 100%) as a white glass. IR (neat) 2939, 2876, 1735, 1447, 1377, 1247, 1074, 1057, 1039 cm⁻¹; ¹H NMR (CDCl₃, 300 MHz) δ 5.10 (t, J=2.70 Hz, 1H), 4.92 (d, J=2.69 Hz, 1H), 4.63−4.52 (m, 1H), 3.98−3.80 (m, 4H), 2.32 (t, J=9.51 Hz, 1H), 2.13 (s, 3H), 2.08 (s, 3H), 2.05 (s, 3H), 2.00−1.40 (series of multiplets, 15H), 1.34−0.98 (m, 3H), 1.20 (s, 3H), 0.92 (s, 3H), 0.82 (s, 3H); ¹³C NMR (CDCl₃, 75 MHz) δ 170.69, 170.63, 170.47, 111.38, 75.07, 74.23, 70.85, 64.95, 63.43, 49.85, 44.73, 43.39, 41.11, 37.37, 34.84, 34.80, 34.52, 31.42, 29.18, 27.02, 25.41, 24.16, 22.72, 22.57, 22.44, 21.73, 21.63, 13.40; HRFAB-MS (thioglycerol+Na⁺ matrix) m/e: ([M+H]⁺) 521.3106 (38.6%), calcd. 521.3114. The triacetate (1.09 g, 2.10 mmol) was dissolved in MeOH (50 mL). NaOH (0.84 g, 21 mmol) was added to the solution. The suspension was then refluxed under N₂ for 24 hours. MeOH was then removed in vacuo and the residue was dissolved in Et₂ O (100 mL) and washed with H₂ O, brine, and then dried over anhydrous Na₂ SO₄. The desired product (0.80 g, 96% yield) was obtained as white solid after removal of solvent. m.p. 199-200° C. IR (neat) 3396, 2932, 1462, 1446, 1371, 1265, 1078, 1055 cm⁻¹; ¹H NMR (10% CD₃ OD in CDCl₃, 300 MHz) δ 4.08−3.83 (series of multiplets, 9H), 3.44−3.34 (m, 1H), 2.41 (t, J=9.28 Hz, 1H), 2.22−2.10 (m, 2H), 1.96−1.50 (series of multiplets, 12H), 1.45−0.96 (series of multiplets, 4H), 1.32 (s, 3H), 0.89 (s, 3H), 0.78 (s, 3H); ¹³C NMR (10% CD₃OD in CDCl₃, 75 MHz) δ 112.11, 72.35, 71.57, 68.09, 64.54, 63.24, 49.36, 45.90, 41.48, 41.45, 39.18, 38.79, 35.29, 34.71, 34.45, 29.90, 27.26, 26.60, 23.65, 22.54, 22.44, 22.35, 13.46; HRFAB-MS (thioglycerol+Na⁺ matrix) m/e: ([M+Na]⁺) 417.2622 (87.3%), calcd. 417.2617.

Compound 44: To a round-bottom flask were added 43 (0.80 g, 2.03 mmol) and dry THF (100 mL) followed by the addition of NaH (60% in mineral oil, 0.81 g, 20.3 mmol). The suspension was refluxed under N₂ for 30 minutes before the addition of allyl bromide (1.75 mL, 20.3 mmol). After 48 hours of reflux, another 10 eq. of NaH and allyl bromide were added. After another 48 hours, TLC showed no intermediates left. Cold water (50 mL) was added to the cooled suspension. The resulted mixture was extracted with Et₂O (60 mL, 2×30 mL). The combined extracts were washed with brine and dried over anhydrous Na₂SO₄. SiO₂ column chromatography (6% EtOAc in hexanes) gave the desired product (0.94 g, 90% yield) as a pale yellow oil. IR (neat) 3076, 2933, 2866, 1645, 1446, 1423, 1408, 1368, 1289, 1252, 1226, 1206, 1130, 1080, 1057 cm⁻¹; ¹H NMR (CDCl₃, 300 MHz) δ 6.02−5.84 (m, 3H), 5.31−5.04 (m, 6H), 4.12−4.05 (m, 2H), 4.01−3.81 (m, 7H), 3.70 (dd, J=12.94, 5.62 Hz, 1H), 3.55 (t, J=2.56 Hz, 1H), 3.33 (d, J=2.93 Hz, 1H), 3.18−3.08 (m, 1H), 2.65 (t, J=10.01 Hz, 1H), 2.32−2.14 (m, 3H), 1.84−1.45 (series of multiplets, 10H), 1.41−1.22 (m, 3H), 1.27 (s, 3H), 1.14−0.92 (m, 2H), 0.89 (s, 3H), 0.75 (s, 3H); ¹³C NMR (CDCl₃, 75 MHz) δ 136.38, 136.07, 136.00, 116.31, 115.54, 115.38, 112.34, 80.07, 79.22, 75.05, 69.83, 69.34, 68.82, 65.14, 63.24, 48.80, 45.96, 42.47, 42.15, 39.40, 35.55, 35.16, 35.15, 29.04, 28.22, 27.52, 24.21, 23.38, 23.11, 22.95, 22.58, 13.79; HRFAB-MS (thioglycerol+Na⁺ matrix) m/e: ([M+Na]⁺) 537.3549 (100%), calcd. 537.3556.

Compound 45: To the solution of 44 (0.94 g, 1.83 mmol) in dry THF (50 mL) was added 9-BBN (0.5 M solution in THF, 14.7 mL, 7.34 mmol) and the mixture was stirred under N₂ at room temperature for 12 hours before the addition of 20% NaOH solution (4 mL) and 30% H₂ O₂ solution (4 mL). The resulted mixture was then refluxed for an hour followed by the addition of brine (100 mL) and extracted with EtOAc (4×30 mL). The combined extracts were dried over anhydrous Na₂SO₄. After the removal of solvent, the residue was purified by SiO₂ column chromatography (EtOAc followed by 10% MeOH in CH₂Cl₂) to give the product (0.559 g, 54% yield) as a colorless oil. IR (neat) 3410, 2933, 2872, 1471, 1446, 1367, 1252, 1086 cm⁻¹; ¹H NMR (CDCl₃, 300 MHz) δ 4.02−3.52 (series of multiplets, 17H), 3.41−3.35 (m, 1H), 3.29 (d, J=2.44 Hz, 1H), 3.22−3.15 (m, 3H), 2.58 (t, J=10.01 Hz, 1H), 2.27−1.95 (m, 3H), 1.83−1.48 (series of multiplets, 16H), 1.40−0.93 (series of multiplets, 5H), 1.27 (s, 3H), 0.90 (s, 3H), 0.75 (s, 3H); ¹³C NMR (CDCl₃, 75 MHz) δ 1112.41, 80.09, 79.09, 76.31, 66.70, 66.02, 65.93, 64.80, 63.26, 61.53, 61.25, 60.86, 48.59, 45.80, 42.51, 41.72, 39.10, 35.36, 35.02, 34.98, 32.87, 32.52, 32.40, 28.88, 27.94, 27.21, 24.33, 23.02, 22.84 (2 C's), 22.44, 13.69; HRFAB-MS (thioglycerol+Na⁺ matrix) m/e: ([M+Na]⁺) 591.3881 (100%), calcd. 591.3873.

Compound 46: To a solution of 45 (0.559 g, 0.98 mmol) in acetone (40 mL) and water (4 mL) was added PPTS (0.124 g, 0.49 mmol) and the solution was refluxed under N₂ for 16 hours. The solvent was removed under reduced pressure. Water (40 mL) was then added to the residue and the mixture was extracted with EtOAc (40 mL, 2×20 mL). The combined extracts were washed with brine, dried and evaporated to dryness. SiO₂ column chromatography (8% MeOH in CH₂Cl₂) of the residue afforded the desired product (0.509 g, 98% yield) as clear oil. IR (neat) 3382, 2941, 2876, 1699, 1449, 1366, 1099 cm⁻¹; ¹H NMR (CDCl₃, 300 MHz) δ 3.83−3.72 (m, 8H), 3.66 (t, J=5.62 Hz, 2H), 3.54 (bs, 2H), 3.43−3.28 (m, 4H), 3.24−3.12 (m, 2H), 2.26−2.00 (m, 4H), 2.08 (s, 3H), 1.98−1.50 (series of multiplets, 15H), 1.42−0.96 (series of multiplets, 6H), 0.90 (s, 3H), 0.62 (s, 3H); ¹³C NMR (CDCl₃, 75 MHz) δ 210.49, 78.87 (2 C's), 76.30, 66.86, 66.18, 65.69, 61.74, 61.43, 60.71, 55.31, 48.05, 43.02, 41.58, 39.53, 35.28, 35.09, 34.96, 32.77, 32.70, 32.31, 31.12, 28.72, 27.88, 27.14, 23.47, 22.75, 22.47, 22.34, 13.86; HRFAB-MS (thioglycerol+Na⁺ matrix) m/e: ([M+Na]⁺) 547.3624 (100%), calcd. 547.3611.

Compound 47: To a solution of 46 (0.18 g, 0.344 mmol) in dry CH₂Cl₂ (10 mL) at 0° C. was added Et₃ N (0.168 mL, 1.20 mmol) followed by the addition of mesyl chloride (0.088 mL, 1.13 mmol). After 10 minutes, H₂O (3 mL) and brine (30 mL) were added. The mixture was extracted with EtOAc (30 mL, 2×10 mL) and the extracts were washed with brine and dried over anhydrous Na₂ SO₄. After removal of solvent, the residue was dissolved in DMSO (5 mL) and NaN₃ (0.233 g, 3.44 mmol). The suspension was heated up to 50° C. under N₂ for 12 hours. H₂ O (50 mL) was added to the cool suspension and the mixture was extracted with EtOAc (30 mL, 2×10 mL) and the extracts were washed with brine and dried over anhydrous Na₂ SO₄—SiO₂ column chromatography (EtOAc/hexanes 1:5) afforded the product (0.191 g, 88% yield for two steps) as a pale yellow oil. IR (neat) 2933, 2872, 2096, 1702, 1451, 1363, 1263, 1102 cm⁻¹; ¹H NMR (CDCl₃, 300 MHz) δ 3.72−3.64 (m, 2H), 3.55−3.24 (series of multiplets, 11H), 3.18−3.02 (m, 2H), 2.22−2.02 (m, 4H), 2.08 (s, 3H), 1.95−1.46 (series of multiplets, 15H), 1.38−0.96 (series of multiplets, 6H), 0.89 (s, 3H), 0.62 (s, 3H); ¹³C NMR (CDCl₃, 75 MHz) δ 210.36, 79.69, 79.22, 75.98, 65.08, 64.80, 64.53, 55.31, 48.93, 48.86, 48.76, 48.06, 43.03, 41.91, 39.66, 35.44, 35.31, 35.12, 31.04, 29.77, 29.69, 29.67, 28.99, 28.10, 27.65, 23.60, 22.99, 22.95, 22.50, 14.00; HRFAB-MS (thioglycerol+Na⁺ matrix) m/e: ([M+Na]⁺) 622.3820 (100%), calcd. 622.3805.

Compound CSA-11: Compound 47 (0.191 g, 0.319 mmol) was dissolved in dry THF (20 mL) followed by the addition of LiAlH₄ (60.4 mg, 1.59 mmol). The grey suspension was stirred under N₂ at room temperature for 12 hours. Na₂SO₄. 10H₂O powder was carefully added. After the grey color in the suspension disappeared, anhydrous Na₂SO₄ was added and the precipitate was filtered out. After the removal of solvent, the residue was purified by column chromatography (silica gel, MeOH/CH₂Cl₂/28% NH₃.H₂ O 3:3:1). After most of the solvent was rotavapped off from the fractions collected, 5% HCl solution (2 mL) was added to dissolve the milky residue. The resulted clear solution was then extracted with Et₂O (2×10 mL). 20% NaOH solution was then added until the solution became strongly basic. CH₂Cl₂ (20 mL, 2×10 mL) was used to extract the basic solution. The combined extracts were dried over anhydrous Na₂SO₄ and removal of solvent gave the desired product (0.115 g, 69% yield) as a colorless oil. From ¹H NMR it appears that this compound was a mixture of two stereoisomers at C₂₀ with a ratio of approximately 9:1. The stereoisomers were not separated, but used as recovered. Spectra for the most abundant isomer: IR (neat) 3353, 2926, 2858, 1574, 1470, 1366, 1102 cm⁻¹; ¹H NMR (20% CDCl₃ in CD₃ OD, 300 MHz) δ 4.69 (bs, 7H), 3.76−3.69 (m, 1H), 3.63−3.53 (m, 5H), 3.50−3.40 (m, 1H), 3.29 (bs, 1H), 3.18−3.07 (m, 2H), 2.94−2.83 (m, 1H), 2.81−2.66 (m, 5H), 2.23−2.06 (m, 4H), 1.87−1.50 (series of multiplets, 15H), 1.39−0.96 (series of multiplets, 6H), 1.11 (d, J=6.10 Hz, 3H), 0.93 (s, 3H), 0.75 (s, 3H); ¹³C NMR (20% CDCl₃ in CD₃ OD, 75 MHz) δ 81.46, 80.67, 77.32, 70.68, 67.90, 67.66, 67.18, 50.32, 47.17, 43.30, 43.06, 40.74, 40.64, 40.38, 40.26, 36.31, 36.28, 35.93, 34.30, 34.02, 33.29, 29.63, 29.31, 28.43, 26.10, 24.67, 24.09, 23.96, 23.50, 13.30 for the major isomer; HRFAB-MS (thioglycerol+Na⁺ matrix) m/e: ([M+H]⁺) 524.4431 (64.2%), calcd. 524.4427.

### Example 6

This example includes a description of one or more exemplary synthestic procedures for obtaining Compounds CSA-10 and 48-49.

Compound 48: To a solution of 23 (0.15 g, 0.233 mmol) in dry CH₂ Cl₂ (15 mL) at 0° C. was added Et₃ N (48.8 μL, 0.35 mmol) followed by the addition of CH₃SO₂Cl (21.7 μL, 0.28 mmol). The mixture was stirred for 15 minutes before H₂O (3 mL) was added. Saturated NaCl solution (20 mL) was then added, and the mixture was extracted with EtOAc (40 mL, 2×20 mL). The combined extracts were washed with brine and dried over anhydrous Na₂SO₄. The solvent was rotovapped off and to the residue were added NaBr (0.12 g, 1.17 mmol) and DMF (10 mL). The suspension was heated up to 80° C. under N₂ for 2 hours. DMF was removed under vacuum and the residue was chromatographed on silica (EtOAc/hexanes 1:10) to give the desired product (0.191 g, 97% yield) as a pale yellow oil. ¹H NMR (CDCl₃, 300 MHz) δ 3.69−3.35 (series of multiplets, 13H), 3.28−3.02 (series of multiplets, 4H), 2.18−2.04 (m, 3H), 2.00−1.60 (series of multiplets, 16H), 1.58−0.96 (series of multiplets, 11H), 0.92 (d, J=6.34 Hz, 3H), 0.89 (s, 3H), 0.66 (s, 3H); ¹³C NMR (CDCl₃, 75 MHz) δ 80.62, 79.81, 76.08, 65.07, 64.50, 64.34, 49.03, 48.98, 48.79, 46.49, 46.46, 42.73, 42.02, 39.85, 35.47, 35.34, 35.12, 34.79, 34.72, 29.82, 29.80, 29.74, 29.11, 27.91, 27.78, 27.69, 23.55, 23.07, 22.88, 18.10, 12.62; HRFAB-MS (thioglycerol+Na⁺ matrix) m/e: ([M−H]⁺) 706.3609 (63.1%), calcd. 706.3591; 704.3616 (52.8%), calcd. 704.3611.

Compound 49: Compound 48 (0.191 g, 0.269 mmol) and 23 (0.295 g, 0.459 mmol) was dissolved in DMF (3 mL, distilled over BaO at 6 mm Hg before use) followed by the addition of NaH (0.054 g, 60% in mineral oil). The suspension was stirred under N₂ at room temperature for 24 hours. H₂ O (100 mL) was added to quench excess NaH and the mixture was then extracted with Et₂ O (40 mL, 3×20 mL) and the combined extracts were washed with brine and dried over anhydrous Na₂ SO₄. The desired product (0.177 g, 52% yield based on compound 23) was obtained as a pale yellow oil after SiO₂ chromatography (EtOAc/hexanes 1:6, then 1:2). IR (neat) 2940, 2862, 2095, 1472, 1456, 1362, 1263, 1113 cm¹; ¹H NMR(CDCl₃, 300 MHz) δ 3.68−3.35 (series of multiplets, 26H), 3.28−3.02 (series of multiplets, 8H), 2.20−2.04 (m, 6H), 1.96−1.60 (series of multiplets, 30H), 1.52−0.98 (series of multiplets, 12H), 0.91 (d, J=6.59 Hz, 6H), 0.89 (s, 6H), 0.65 (s, 6H); ¹³C NMR(CDCl₃, 75 MHz) δ 80.68, 79.83, 76.13, 71.71, 65.06, 64.48, 64.39, 49.08, 48.98, 48.80, 46.64, 46.44, 42.71, 42.04, 39.88, 35.73, 35.49, 35.36, 35.14, 32.41, 29.84, 29.81, 29.76, 29.14, 27.92, 27.78, 27.69, 26.58, 23.59, 23.08, 22.92, 18.12, 12.64.

Compound CSA-10: Compound 49 (0.219 g, 0.173 mmol) was dissolved in dry THF (10 mL) followed by the addition of LiAlH₄ (65 mg, 1.73 mmol). The grey suspension was stirred under N₂ at room temperature for 12 hours. Na₂SO₄. 10H₂O powder was carefully added. After the grey color in the suspension disappeared, anhydrous Na₂SO₄ was added and the precipitate was filtered out. After the removal of solvent, the residue was purified by column chromatography (silica gel, MeOH/CH₂Cl₂/28% NH₃.H₂O 2.5:2.5:1). After most of the solvent was rotavapped off from the fractions collected, 5% HCl solution (2 mL) was added to dissolve the milky residue. The resulted clear solution was then extracted with Et₂O (2×10 mL). 20% NaOH solution was then added until the solution became strongly basic. CH₂Cl₂ (20 mL, 2×10 mL) was used to extract the basic solution. The combined extracts were dried over anhydrous Na₂SO₄ and removal of solvent gave the desired product (0.147 g, 76% yield) as a white glass. IR (neat) 3364, 3287, 2934, 2861, 1596, 1464, 1363, 1105 cm⁻¹; ¹H NMR (20% CDCl₃ in CD₃OD, 500 MHz) δ 4.74 (bs, 12H), 3.75−3.70 (m, 2H), 3.65−3.61 (m, 2H), 3.57−3.52 (m, 6H), 3.40 (t, J=3.60 Hz, 4H), 3.30 (bs, 4H), 3.16−3.10 (m, 4H), 2.84−2.73 (m, 12H), 2.18−2.07 (m, 6H), 1.97−1.61 (series of multiplets, 30H), 1.58−0.98 (series of multiplets, 24H), 0.95 (d, J=6.84 Hz, 6H), 0.94 (s, 6H), 0.70 (s, 6H); ¹³C NMR (20% CDCl₃ in CD₃OD, 125 MHz) δ 81.70, 80.52, 77.09, 72.34, 67.75 (2 C's), 67.07, 47.80, 47.13, 43.76, 42.87, 41.20, 40.65, 40.58, 40.14, 36.43, 36.25, 36.08, 35.77, 34.15, 33.87 (2 C's), 33.18, 29.55, 28.92, 28.47, 28.42, 27.25, 24.27, 23.54, 23.41, 18.70, 13.07; HRFAB-MS (thioglycerol+Na⁺ matrix) m/e: ([M+H]⁺) 113.9625 (68.8%), calcd. 1113.9610.

### Example 7

This example includes a description of one or more exemplary synthestic procedures for obtaining Compounds 111-113 and 116a-d.

Compounds 116a-d: Representative procedure: preparation of 116b. NaH (0.06 g, 60% in mineral oil, 1.49 mmol) and propyl bromide (0.136 mL, 1.49 mmol) were added to a DMF solution of compound 23 (described in Li et al., J. Am. Chem. Soc. 1998, 120, 2961) (0.096 g, 0.149 mmol). The suspension was stirred under N₂ for 24 hr. H₂O (20 mL) was added, and the mixture was extracted with hexanes (3×10 mL). The combined extracts were dried over Na₂SO₄ and concentrated in vacuo. Silica gel chromatography (10% EtOAc in hexanes) afforded the desired product (92 mg, 90% yield) as a pale yellow oil. ¹H NMR (CDCl₃, 500 MHz) δ 3.68−3.64 (m, 1H), 3.61−3.57 (m, 1H), 3.52 (t, J=6.1 Hz, 2H), 3.49 (bs, 1H), 3.46−3.35 (m, 10H), 3.25 (d, J=2.4 Hz, 1H), 3.23−3.19 (m, 1H), 3.16−3.11 (m, 1H), 3.09−3.03 (m, 1H), 2.17−2.03 (m, 3H), 1.95−1.55 (m, 17H), 1.51−1.40 (m, 4H), 1.38−1.17 (m, 5H), 1.11−0.96 (m, 3H), 0.93−0.89 (m, 9H), 0.65 (s, 3H); ¹³C NMR (CDCl₃, 75 MHz) δ 80.64, 79.79, 76.08, 72.67, 71.59, 65.01, 64.44, 64.33, 49.04, 48.94, 48.75, 46.61, 46.40, 42.68, 42.00, 39.83, 35.72, 35.45, 35.30, 35.10, 32.38, 29.81, 29.77, 29.72, 29.09, 27.88, 27.76, 27.65, 26.52, 23.55, 23.12, 23.04, 22.87, 18.06, 12.60, 10.79; HRFAB-MS (thioglycerol+Na⁺ matrix) m/e: ([M+Na]⁺) 708.4910 (23.5%), calcd. 708.4920.

Compounds 111, CSA-17, and 113: Representative procedure: preparation of CSA-17. Compound 116b (0.092 g, 0.134 mmol) was dissolved in THF (10 mL) followed by the addition of LiAlH₄ (0.031 g, 0.81 mmol). The suspension was stirred under N₂ for 12 hr. Na₂SO₄.10H₂ O (about 1 g) was then carefully added. After the gray color in the suspension dissipated, anhydrous Na₂SO₄ was added, and the precipitate was removed by filtration. Concentration and silica gel chromatography (CH₂Cl₂/MeOH/28% NH₃.H₂O 12:6:1, then 10:5:1) yielded a glass which was dissolved in 1 M HCl (2 mL). The resulting clear solution was washed with Et₂O (2×10 mL). 20% NaOH solution was added to the aqueous phase until the solution became strongly basic. CH₂Cl₂ (3×10 mL) was used to extract the basic solution. The combined extracts were dried over anhydrous Na₂SO₄ and concentrated in vacuo to give the desired product (0.045 g, 55% yield) as a white glass. ¹H NMR (about 20% CDCl₃ in CD₃OD, 500 MHz) δ 4.73 (bs, 6H), 3.74−3.70 (m, 1H), 3.65−3.61 (m, 1H), 3.55 (t, J=6.3 Hz, 2H), 3.42−3.38 (m, 4H), 3.33−3.30 (m, 2H), 3.16−3.10 (m, 2H), 2.83−2.73 (m, 6H), 2.18−2.06 (m, 3H), 1.96−1.20 (series of multiplets, 26H), 1.12−0.98 (m, 3H), 0.95−0.92 (m, 9H), 0.70 (s, 3H); ¹³C NMR (about 20% CDCl₃ in CD₃OD, 75 MHz) δ 81.67, 80.49, 77.04, 73.44, 72.28, 67.77, 67.71, 67.06, 47.74, 47.08, 43.75, 42.82, 41.21, 40.60, 40.56, 40.12, 36.47, 36.19, 36.04, 35.74, 34.09, 33.82, 33.78, 33.16, 29.49, 28.87, 28.43, 27.18, 24.22, 23.66, 23.49, 23.40, 18.64, 13.04, 11.03; HRFAB-MS (thioglycerol+Na⁺ matrix) m/e: ([M+H]⁺) 608.5348 (100%), calcd. 608.5330. 111: ¹H NMR (about 20% CDCl₃ in CD₃OD, 500 MHz) δ 4.79 (bs, 6H), 3.74−3.71 (m, 1H), 3.66−3.62 (m, 1H), 3.55 (t, J=6.1 Hz, 2H), 3.52 (bs, 1H), 3.38−3.28 (series of multiplets, 4H), 3.33 (s, 3H), 3.16−3.10 (m, 2H), 2.83−2.72 (m, 6H), 2.19−2.07 (m, 3H), 1.97−1.62 (series of multiplets, 15H), 1.58−1.20 (series of multiplets, 9H), 1.13−0.98 (m, 3H), 0.95 (d, J=6.3 Hz, 3H), 0.93 (s, 3H), 0.70 (s, 3H); ¹³C NMR (about 20% CDCl₃ in CD₃OD, 75 MHz) δ 81.82, 80.65, 77.20, 74.43, 67.85, 67.18, 58.90, 47.80, 47.22, 43.91, 43.01, 41.31, 40.78, 40.69, 40.22, 36.63, 36.35, 36.18, 35.86, 34.27, 33.97, 33.26, 29.60, 29.03, 28.58, 28.53, 27.14, 24.33, 23.61, 23.45, 18.68, 13.06; HRFAB-MS (thioglycerol+Na⁺ matrix) m/e: ([M+Na]⁺) 602.4855 (100%), calcd. 602.4873. 113: ¹H NMR (about 50% CDCl₃ in CD₃OD, 500 MHz) δ 4.08 (bs, 6H), 3.71−3.67 (m, 1H), 3.62−3.58 (m, 1H), 3.53 (t, J=6.3 Hz, 2H), 3.49 (bs, 1H), 3.43−3.38 (m, 4H), 3.31−3.27 (m, 2H), 3.14−3.07 (m, 2H), 2.83−2.73 (m, 6H), 2.16−2.03 (m, 3H), 1.93−1.17 (series of multiplets, 30H), 1.10−0.96 (m, 3H), 0.93−0.89 (m, 9H), 0.67 (s, 3H); ¹³C NMR (about 50% CDCl₃ in CD₃OD, 75 MHz) δ 80.51, 79.35, 75.85, 71.29, 70.83, 66.73, 66.62, 65.96, 46.68, 45.98, 42.59, 41.63, 40.20, 39.53, 39.43, 39.21, 35.34, 35.04, 35.00, 34.71, 33.11, 32.90, 32.82, 32.00, 29.15, 28.49, 28.15, 27.75, 27.35, 26.22, 23.18, 22.60, 22.45, 22.34, 17.77, 13.75, 12.22; HRFAB-MS (thioglycerol+Na⁺ matrix) m/e: ([M+H]⁺) 636.5679 (100%), calcd. 636.5669.

### Example 8

This example includes a description of one or more exemplary synthestic procedures for obtaining Compounds 106 and 124.

Compound 124: Compound 47 (0.256 g, 0.489 mmol) was dissolved in CH₂Cl₂ (10 mL), and cooled to 0° C. followed by the addition of Na₂HPO₄ (0.69 g, 4.89 mmol) and urea-hydrogen peroxide complex (UHP) (0.069 g, 0.733 mmol). Trifluoroacetic anhydride (TFAA) (0.138 mL, 0.977 mmol) was then added dropwise. The suspension was stirred for 12 hr, and additional UHP (23 mg, 0.25 mmol) and TFAA (0.069 mL, 0.49 mmol) were added. After another 12 hr, H₂O (30 mL) was added, and the resulting mixture was extracted with EtOAc (3×20 mL). The combined extracts were washed with brine (50 mL), dried over anhydrous Na₂SO₄, and concentrated in vacuo. SiO₂ chromatography (EtOAc/hexanes 1:5) afforded the desired product (0.145 g, 55% yield) as a colorless oil. ¹H NMR (CDCl₃, 300 MHz) δ 5.21 (dd, J=9.3 and 7.3 Hz, 1H), 3.70−3.57 (m, 2H), 3.55 (t, J=6.0 Hz, 2H), 3.43−3.37 (m, 6H), 3.32−3.25 (m, 3H), 3.17−3.02 (m, 2H), 2.28−2.05 (m, 4H), 2.03 (s, 3H), 1.86−1.19 (series of multiplets, 19H), 0.97 (dd, J=14.5 and 3.3 Hz, 1H), 0.90 (s, 3H), 0.78 (s, 3H); ¹³C NMR (CDCl₃, 75 MHz) δ 171.08, 79.71, 78.03, 75.72, 75.53, 65.41, 65.04, 64.53, 48.79, 48.70, 46.49, 41.92, 39.44, 37.81, 35.45, 35.22, 35.10, 29.73, 29.63, 28.89, 28.33, 27.50, 27.34, 23.39, 22.97, 22.92, 21.28, 12.72; HRFAB-MS (thioglycerol+Na⁺ matrix) m/e: ([M−H]⁺) 614.3798 (24.5%), calcd. δ 14.3778.

Compound 106: Compound 124 (0.145 g, 0.236 mmol) was dissolved in CH₂Cl₂ (2 mL) and MeOH (1 mL). 20% NaOH solution (0.2 mL) was added. The mixture was stirred for 12 hr, and anhydrous Na₂SO₄ was used to remove water. After concentration in vacuo, the residue was purified by silica gel chromatography (EtOAc/hexanes 1:3) to afford the desired product (0.124 g, 92% yield) as a colorless oil. ¹H NMR (CDCl₃, 300 MHz) δ 4.29 (bs, 1H), 3.69−3.60 (m, 2H), 3.52 (t, J=6.0 Hz, 2H), 3.45−3.32 (m, 8H), 3.26 (d, J=2.7 Hz, 1H), 3.17−3.02 (m, 2H), 2.19−1.94 (m, 4H), 1.90−1.62 (series of multiplets, 13H), 1.57−1.20 (series of multiplets, 7H), 0.97 (dd, J=14.3 and 3.1 Hz, 1H), 0.90 (s, 3H), 0.73 (s, 3H); ¹³C NMR (CDCl₃, 75 MHz) δ 79.69, 78.03, 75.47, 73.38, 65.46, 65.00, 64.47, 48.87, 48.68, 46.83, 41.93, 39.71, 37.87, 35.43, 35.20, 35.09, 29.96, 29.69, 29.59, 29.53, 28.89, 28.44, 27.48, 23.72, 22.91, 22.71, 11.77. The alcohol (0.124 g, 0.216 mmol) was dissolved in dry THF (20 mL) followed by the addition of LiAlH₄ (33 mg, 0.866 mmol). The gray suspension was stirred under N₂ for 12 hr. Na₂SO₄.10H₂O (about 2 g) was carefully added. After the gray color in the suspension dissipated, anhydrous Na₂SO₄ was added and the precipitate was removed by filtration. After the removal of solvent, the residue was purified by column chromatography (SiO₂, MeOH/CH₂Cl₂/28% NH₃.H₂O 2.5:2.5:1). After concentration of the relevant fractions, 1 M HCl (2 mL) was added to dissolve the milky residue. The resulting clear solution was washed with Et₂O (2×10 mL). To the aqueous phase, 20% NaOH solution was added until the solution became strongly basic. CH₂Cl₂ (20 mL, 2×10 mL) was used to extract the basic solution. The combined extracts were dried over anhydrous Na₂SO₄ and removal of solvent gave the desired product (0.050 g, 47% yield) as a colorless oil. ¹H NMR (20% CDCl₃ in CD₃OD, 300 MHz) δ 4.77 (s, 7H), 4.25 (t, J=8.5 Hz, 1H), 3.75−3.68 (m, 1H), 3.66−3.58 (m, 1H), 3.55 (t, J=6.1 Hz, 2H), 3.48−3.41 (m, 1H), 3.34 (bs, 1H), 3.30 (d, J=3.6 Hz, 1H), 3.17−3.08 (m, 2H), 2.86−2.70 (m, 6H), 2.20−1.91 (m, 4H), 1.88−1.16 (series of multiplets, 19H), 1.00 (dd, J=14.2 and 3.0 Hz, 1H), 0.93 (s, 3H), 0.73 (s, 3H); ¹³C NMR (20% CDCl₃ in CD₃OD, 75 MHz) δ 80.62, 79.12, 76.74, 73.77, 68.50, 67.79, 67.17, 47.69, 43.04, 40.76, 40.64, 40.62, 40.22, 39.01, 36.32, 36.25, 35.94, 34.27, 33.97, 33.72, 30.13, 29.53, 28.43, 24.48, 23.58, 23.40, 12.38; HRFAB-MS (thioglycerol+Na⁺ matrix) m/e: ([M+H]⁺) 496.4108 (100%), calcd. 496.4114.

### Example 9

This example includes a description of one or more exemplary synthestic procedures for obtaining Compounds 109 and 126-129.

Compound 126: Compound 125 (2.30 g, 3.52 mmol) was dissolved in MeOH (50 mL) and CH₂Cl₂ (100 mL). A small amount of Et₃N was added, and the solution was cooled to −78° C. Ozone was bubbled through the solution until a blue color persisted. Me₂S (4 mL) was introduced followed by the addition of NaBH₄ (0.266 g, 0.703 mmol) in MeOH (10 mL). The resulting solution was allowed to warm and stir overnight. The solution was concentrated in vacuo, and brine (60 mL) was added. The mixture was extracted with EtOAc (40 ml, 2×30 mL), and the combined extracts were washed with brine and dried over anhydrous Na₂SO₄. Silica gel chromatography (EtOAc) afforded the product (1.24 g, 76% yield) as a white solid. m.p. 219-220 C.; ¹H NMR (CDCl₃, 300 MHz) δ 5.10 (t, J=2.8 Hz, 1H), 4.90 (d, J=2.7 Hz, 1H), 3.73−3.59 (m, 2H), 3.56−3.44 (m, 1H), 2.13 (s, 3H), 2.09 (s, 3H), 2.07−0.95 (series of multiplets, 23H), 0.91 (s, 3H), 0.83 (d, J=6.3 Hz, 3H), 0.74 (s, 3H); ¹³C NMR (CDCl₃, 75 MHz) δ 170.84, 170.82, 75.63, 71.77, 71.03, 60.73, 48.10, 45.26, 43.54, 41.16, 38.78, 37.89, 35.00, 34.43, 32.26, 31.50, 30.60, 29.07, 27.50, 25.70, 22.96, 22.71, 21.81, 21.63, 18.18, 12.35; HRFAB-MS (thioglycerol+Na⁺ matrix) m/e: ([M+H]⁺) 465.3197 (20%), calcd. 465.3216.

Compound 127: Compound 126 (1.24 g, 2.67 mmol) was dissolved in MeOH (30 mL), and NaOH (0.54 g, 13.4 mmol) was added. The suspension was refluxed under N₂ for 24 hr. The MeOH was removed in vacuo followed by the addition of H₂O (50 mL). The precipitate was filtered, washed with H₂O and then dried in vacuo to give a white solid (1.02 g). This solid was dissolved in DMF (40 mL) followed by the sequential addition of NEt₃ (1.12 mL, 8.02 mmol), DMAP (16.3 mg, 0.13 mmol) and trityl chloride (1.49 g, 5.34 mmol). The suspension was stirred under N₂ for 12 hr and then heated up to 50° C. for 24 hr. H₂O (100 mL) was added to the cooled suspension, and the mixture was extracted with EtOAc (3×50 mL). The combined extracts were washed with brine (100 mL), dried over anhydrous Na₂SO₄, and concentrated in vacuo. Silica gel chromatography (EtOAc) afforded the product (1.20 g, 72% yield) as a pale yellow glass. To this glass was added dry THF (80 mL) and NaH (60% in mineral oil, 0.77 g, 19.3 mmol). The suspension was refluxed under N₂ for half an hour before the introduction of allylbromide (1.67 mL, 19.3 mmol). After 48 hr at reflux, another 10 eq. of NaH and allylbromide were introduced. After another 48 hr, the reaction mixture was cooled and H₂O (100 mL) was slowly added. The resulting mixture was extracted with hexanes (3×50 mL), and the combined extracts were washed with brine (100 mL) and dried over anhydrous Na₂SO₄. Silica gel chromatography (5% EtOAc in hexanes) afforded the product (1.27 g, 64% yield for all three steps) as a clear glass. ¹H NMR (CDCl₃, 300 MHz) δ 7.46−7.43 (m, 6H), 7.29−7.16 (m, 9H), 5.98−5.81 (m, 3H), 5.29−5.18 (m, 3H), 5.14−5.03 (m, 3H), 4.11−3.97 (m, 4H), 3.75−3.67 (m, 2H), 3.49 (bs, 1H), 3.32−3.13 (d, J=2.4 Hz, 1H), 3.20−3.13 (m, 2H), 3.00 (m, 1H), 2.33−2.12 (m, 3H), 2.03−0.92 (series of multiplets, 19H), 0.88 (s, 3H), 0.78 (d, J=6.6 Hz, 3H), 0.65 (s, 3H); ¹³C NMR (CDCl₃, 75 MHz) δ 144.71, 136.08, 136.04, 135.94, 128.80, 127.76, 126.86, 116.30, 115.57, 86.53, 80.77, 79.20, 74.96, 69.42, 69.34, 68.81, 62.00, 46.87, 46.48, 42.67, 42.11, 39.90, 36.15, 35.50, 35.14, 35.10, 33.23, 28.99, 28.09, 27.75, 27.56, 23.36, 23.32, 23.12, 18.24, 12.66; HRFAB-MS (thioglycerol+Na⁺ matrix) m/e: ([M+Na]⁺) 765.4875 (100%), calcd. 765.4859.

Compound 128: To a THF (40 mL) solution of 127 (1.27 g, 1.71 mmol) was added 9-BBN (0.5 M solution in THF, 17.1 mL). The mixture was stirred for 12 hr before the addition of NaOH (20% solution, 10 mL) and H₂O₂ (30% solution, 10 mL). The resulted mixture was refluxed for 1 hr followed by the addition of brine (100 mL) and extraction with EtOAc (4×30 mL). The combined extracts were dried over anhydrous Na₂SO₄ and concentrated in vacuo. Silica gel chromatography (5% MeOH in CH₂Cl₂) afforded the product (1.26 g, 93% yield) as a clear glass. ¹H NMR (5% CD₃OD in CDCl₃, 300 MHz) □ 7.46−7.43 (m, 6H), 7.32−7.20 (m, 9H), 3.94 (s, 3H), 3.78−3.56 (m, 10H), 3.48 (bs, 1H), 3.32−3.26 (m, 2H), 3.24−3.12 (m, 3H), 3.00 (dd, J=8.2 and 6.1 Hz, 1H), 2.23−1.96 (m, 3H), 1.90−0.95 (series of multiplets, 25H), 0.90 (s, 3H), 0.77 (d, J=6.6 Hz, 3H), 0.66 (s, 3H); ¹³C NMR (5% CD₃OD in CDCl₃, 75 MHz) δ 144.52, 128.64, 127.64, 126.76, 86.43, 80.55, 79.31, 77.65, 77.23, 76.80, 76.06, 66.17, 66.01, 65.41, 61.93, 61.20, 60.73, 60.39, 47.29, 46.08, 42.65, 41.62, 39.49, 36.02, 35.10, 34.89, 34.77, 32.89, 32.71, 32.41, 32.26, 28.68, 27.70, 27.51, 27.19, 23.26, 22.66, 22.50, 18.23, 12.34; HRFAB-MS (thioglycerol+Na⁺ matrix) m/e: ([M+Na]⁺) 819.5169 (100%), calcd. 819.5099.

Compound 129: To a CH₂Cl₂ (50 mL) solution of compound 128 (1.26 g, 1.58 mmol) at 0° C. was added Et₃N (0.92 mL, 6.60 mmol) followed by mesyl chloride (0.47 mL, 6.05 mmol). After 15 minutes, H₂O (10 mL) was followed by brine (80 mL). The mixture was extracted with EtOAc (60 mL, 2×30 mL) and the combined extracts were dried over anhydrous Na₂SO₄. After removal of solvent in vacuo, the residue was dissolved in DMSO (10 mL) and NaN₃ (1.192 g, 18.3 mmol) was added. The suspension was heated to 60° C. under N₂ overnight. H₂O (100 mL) was added, and the mixture was extracted with EtOAc (3×40 mL). The combined extracts were washed with brine and dried over anhydrous Na₂SO₄. Removal of the solvent in vacuo afforded a pale yellow oil. The oil was dissolved in MeOH (10 mL) and CH₂Cl₂ (20 mL) and TsOH (17.4 mg, 0.092 mmol) was added. After 12 hr, saturated aqueous NaHCO₃ (20 mL) and brine (50 mL) were added and the mixture was extracted with EtOAc (3×40 mL). The combined extracts were washed with brine (50 mL) and dried over anhydrous Na₂SO₄. Silica gel chromatography (EtOAc/hexanes 1:3) afforded the desired product (0.934, 94%) as a pale yellow oil. ¹H NMR (CDCl₃, 500 MHz) δ 3.75−3.70 (m, 1H), 3.68−3.63 (m, 2H), 3.62−3.57 (m, 1H), 3.53 (t, J=6.1 Hz, 2H), 3.50 (bs, 1H), 3.46−3.38 (m, 6H), 3.26 (d, J=2.4 Hz, 1H), 3.24−3.20 (m, 1H), 3.16−3.12 (m, 1H), 3.10−3.04 (m, 1H), 2.17−2.04 (m, 3H), 1.96−1.63 (m, 14H), 1.53−1.45 (m, 3H), 1.35−1.20 (m, 7H), 1.08−1.00 (m, 1H), 0.97−0.88 (m, 1H), 0.94 (d, J=6.8 Hz, 3H), 0.89 (s, 3H), 0.67 (s, 3H); ¹³C NMR (CDCl₃, 75 MHz) δ 80.64, 79.81, 76.06, 65.05, 64.49, 64.34, 61.03, 49.02, 48.98, 48.78, 46.93, 46.53, 42.76, 42.01, 39.83, 39.14, 35.46, 35.33, 35.12, 32.97, 29.79, 29.73, 29.10, 27.90, 27.68, 23.56, 23.06, 22.88, 18.24, 12.60; HRFAB-MS (thioglycerol+Na⁺ matrix) m/e: ([M+Na]⁺) 652.4285 (100%), calcd. 652.4295.

Compound 109: Compound 129 (0.245 g, 0.391 mmol) was dissolved in THF (30 mL) followed by the addition of LiAlH₄ (59 mg, 1.56 mmol). The gray suspension was stirred under N₂ 12 hr. Na₂SO₄.10H₂O powder (about 1 g) was carefully added. After the gray color in the suspension dissipated, anhydrous Na₂SO₄ was added and the precipitate was removed by filtration. After the removal of solvent, the residue was purified by silica gel chromatography (CH₂Cl₂/MeOH/28% NH₃.H₂O 10:5:1 then 10:5:1.5). The solvent was removed from relevant fractions, and 1 M HCl (4 mL) was added to dissolve the residue. The resulting clear solution was extracted with Et₂O (3×10 mL). 20% NaOH solution was added until the solution became strongly basic. CH₂Cl₂ (4×10 mL) was used to extract the basic solution. The combined extracts were dried over anhydrous Na₂SO₄, and removal of solvent in vacuo gave the desired product (0.15 g, 71% yield) as a colorless oil. ¹H NMR (about 20% CD₃OD in CDCl₃, 500 MHz) δ 4.73 (bs, 7H), 3.74−3.70 (m, 1H), 3.65−3.60 (m, 2H), 3.56−3.52 (m, 4H), 3.31−3.28 (m, 2H), 3.16−3.09 (m, 2H), 2.82−2.71 (m, 6H), 2.19−2.06 (m, 3H), 1.97−1.66 (series of multiplets, 15H), 1.58−1.48 (m, 3H), 1.38−0.98 (m, 7H), 0.96 (d, J=6.8 Hz, 3H), 0.93 (s, 3H), 0.71 (s, 3H); ¹³C NMR (about 20% CD₃OD in CDCl₃, 75 MHz) δ 81.80, 80.60, 77.17, 67.88, 67.86, 67.18, 60.73, 48.11, 47.28, 43.93, 42.99, 41.34, 40.76, 40.72, 40.24, 39.70, 36.33, 36.18, 35.86, 34.29, 33.99, 33.96, 33.83, 29.60, 29.00, 28.57, 28.54, 24.33, 23.59, 23.48, 18.86, 13.04; HRFAB-MS (thioglycerol+Na⁺ matrix) m/e: ([M+H]⁺) 552.4756 (100%), calcd. 552.4772.

### Example 10

This example includes a description of one or more exemplary synthestic procedures for obtaining Compounds 108 and 130.

Compound 130: o-NO₂C₆H₄SeCN (0.094 g, 0.21 mmol) and Bu₃P (0.095 mL, 0.38 mmol) were stirred in dry THF (5 mL) at 0° C. for ½ hr followed by the addition of compound 129 (0.10 g, 0.159 mmol) in THF (2 mL). The suspension was stirred for 1 hr followed by the addition of H₂O₂ (30% aqueous solution, 2 mL). The mixture was stirred for 12 hr followed by extraction with hexanes (4×10 mL). The combined extracts were dried over anhydrous Na₂SO₄. The desired product (0.035 g, 36% yield) was obtained as pale yellowish oil after silica gel chromatography (10% EtOAc/hexanes). ¹H NMR (CDCl₃, 500 MHz) δ 5.73−5.66 (ddd, J=17.1, 10.2, 8.3 Hz, 1H), 4.90 (dd, J=17.1, 2.0 Hz, 1H), 4.82 (dd, J=10.2 Hz, 1.96 Hz, 1H), 3.68−3.64 (m, 1H), 3.62−3.58 (m, 1H), 3.54−3.26 (m, 9H), 3.25−3.22 (m, 2H), 3.15−3.11 (m, 1H), 3.10−3.04 (m, 1H), 2.17−1.62 (series of multiplets, 18H), 1.51-1.43 (m, 2H), 1.35−1.18 (m, 4H), 1.06−0.91 (m, 2H), 1.02 (d, J=6.3 Hz, 3H), 0.90 (s, 3H), 0.68 (s, 3H); ¹³C NMR (CDCl₃, 75 MHz) δ 145.50, 111.72, 80.60, 79.82, 76.09, 65.06, 64.50, 64.45, 49.05, 48.97, 48.79, 46.43, 46.13, 42.76, 42.03, 41.30, 39.84, 35.49, 35.34, 35.15, 29.82, 29.80, 29.75, 29.11, 28.00, 27.84, 27.68, 23.56, 23.08, 22.95, 19.79, 12.87; HRFAB-MS (thioglycerol+Na⁺ matrix) m/e: ([M+Na]⁺) 634.4167 (90.6%), calcd. 634.4169.

Compound 108: Compound 130 (0.105 g, 0.172 mmol) was dissolved in CH₂Cl₂ (5 mL) and MeOH (5 mL) at −78° C. O₃ was bubbled into the solution for ca. 20 min. Me₂S (1 mL) was added followed, and the solvent was removed in vacuo. The residue was dissolved in THF (15 mL), and LiAlH₄ (0.033 g, 0.86 mmol) was added. The suspension was stirred for 12 hr. Na₂SO₄.10H₂O (about 2 g) was carefully added. After the gray color of the suspension dissipated, anhydrous Na₂SO₄ was added and the precipitate was removed by filtration. Concentration and silica gel chromatography (CH₂Cl₂/MeOH/28% NH₃.H₂O 10:5:1.5 then 9:6:1.8) yielded a white glass. To this material was added 1 M HCl (4 mL). The resulting clear solution was washed with Et₂O (3×10 mL). 20% NaOH solution was added to the aqueous phase until the solution became strongly basic. CH₂Cl₂ (4×10 mL) was used to extract the basic solution. The combined extracts were dried over anhydrous Na₂SO₄ and removal of solvent gave the desired product (0.063 g, 68% yield) as a colorless oil. ¹H NMR (about 10% CD₃OD in CDCl₃, 500 MHz) δ 4.76 (bs, 7H), 3.75−3.71 (m, 1H), 3.66−3.62 (m, 1H), 3.58−3.52 (m, 4H), 3.33−3.29 (m, 2H), 3.22 (dd, J=10.5 and 7.6 Hz, 1H), 3.15−3.09 (m, 2H), 2.81 (t, J=6.8 Hz, 2H), 2.76−2.71 (m, 4H), 2.19−2.08 (m, 3H), 2.00−1.66 (series of multiplets, 14H), 1.58−1.45 (m, 3H), 1.40−1.08 (m, 5H), 1.03 (d, J=6.8 Hz, 3H), 1.02−0.96 (m, 1H), 0.93 (s, 3H), 0.72 (s, 3H); ¹³C NMR (about 10% CD₃OD in CDCl₃, 75 MHz) δ 81.74, 80.64, 77.23, 67.95, 67.87, 67.18, 47.32, 44.59, 43.72, 43.01, 41.26, 40.80, 40.71, 40.23, 40.02, 36.36, 36.20, 35.87, 34.27, 33.99, 33.90, 29.60, 29.05, 28.58, 28.08, 24.49, 23.62, 23.46, 16.84, 13.12; HRFAB-MS (thioglycerol+Na⁺ matrix) m/e: ([M+H]⁺) 538.4578 (4.7%), calcd. 538.4584.

### Example 11

This example includes a description of one or more exemplary synthestic procedures for obtaining Compounds CSA-21, 133-134 and CSA-15.

Compound CSA-21:Compound 115 (0.118 g, 0.183 mmol) was dissolved in dry CH₂Cl₂ (10 mL), and SO₃ pyridine complex (0.035 g, 0.22 mmol) was added. The suspension was stirred for 12 hr. The solvent was removed in vacuo to give white powder. To the white powder was added 1 M HCl (10 mL) and the resulting mixture was extracted with CH₂Cl₂ (4×10 mL). The combined extracts were dried over anhydrous Na₂SO₄. The desired product (0.11 g, 84%) was obtained as a pale yellow oil after silica gel chromatography (10% MeOH in CH₂Cl₂). ¹H NMR (about 10% CD₃OD in CDCl₃, 500 MHz) δ 4.03 (t, J=6.8 Hz, 2H), 3.69−3.65 (m, 1H), 3.62−3.58 (m, 1H), 3.55 (t, J=6.1 Hz, 2H), 3.51 (bs, 1H), 3.46−3.38 (m, 6H), 3.27 (d, J=2.4 Hz, 1H), 3.26−3.21 (m, 1H), 3.18−3.07 (m, 2H), 2.18−2.03 (m, 3H), 1.95−1.47 (series of multiplets, 19H), 1.40−0.96 (series of multiplets, 9H), 0.92 (d, J=6.8 Hz, 3H), 0.91 (s, 3H), 0.66 (s, 3H); ¹³C NMR (about 10% CD₃OD in CDCl₃, 75 MHz) δ 80.43, 79.68, 75.87, 69.30, 64.82, 64.32, 64.14, 48.78, 48.73, 48.50, 46.44, 46.21, 42.49, 41.76, 39.61, 35.36, 35.17, 35.06, 34.85, 31.73, 29.53, 29.46, 29.44, 28.84, 27.68, 27.48, 27.38, 25.91, 23.30, 22.75, 22.66, 17.70, 12.32; HRFAB-MS (thioglycerol+Na⁺ matrix) m/e: ([M−H+2Na]⁺) 768.3831 (100%), calcd. 768.3843. The azides were reduced by treating the triazide (0.11 g, 0.15 mmol) with Ph₃ P (0.20 g, 0.77 mmol) in THF (10 mL) and H₂O (1 mL). The mixture was stirred for 3 days. The solvent was removed in vacuo, and the residue was purified by silica gel chromatography (CH₂Cl₂/MeOH/28% NH₃.H₂O 12:6:1 then 10:5:1.5) to afford the desired product (0.077 g, 78% yield) as a glass. HCl in Et₂O (1 M, 0.5 mL) was added to the glass to give the corresponding HCl salt. ¹H NMR (about 10% CDCl₃ in CD₃OD, 500 MHz) δ 4.81 (s, 10H), 4.07−3.97 (m, 2H), 3.82 (bs, 1H), 3.71 (bs, 1H), 3.65 (t, J=5.2 Hz, 2H), 3.57 (bs, 1H), 3.37−3.30 (m, 2H), 3.22−3.02 (m, 8H), 2.12−1.71 (series of multiplets, 17H), 1.65−1.01 (series of multiplets, 13H), 0.97 (d, J=6.8 Hz, 3H), 0.94 (s, 3H), 0.73 (s, 3H); ¹³C NMR (about 10% CDCl₃ in CD₃OD, 75 MHz) δ 81.89, 80.58, 77.50, 70.04, 66.71, 66.56, 66.02, 47.11, 46.76, 44.20, 42.66, 40.50, 39.60, 39.40, 36.24, 36.11, 35.89, 35.67, 32.28, 29.38, 29.23, 29.10, 28.94, 28.49, 26.06, 24.21, 23.46, 23.30, 18.50, 12.86; HRFAB-MS (thioglycerol+Na⁺ matrix) m/e: ([M+Na]⁺) 668.4271 (100%), calcd. 668.4258.

Compound CSA-13: The mesylate derived from 23 (0.19 g, 0.264 mmol) was stirred with excess octyl amine (2 mL) at 80° C. for 12 hr. After removal of octylamine in vacuo, the residue was chromatographed (silica gel, EtOAc/hexanes 1:4 with 2% Et₃ N) to afford the desired product (0.19 g, 95% yield) as a pale yellow oil. ¹H NMR (CDCl₃, 300 MHz) δ 3.69−3.37 (series of multiplets, 11H), 3.26−3.00 (m, 4H), 2.61−2.53 (m, 4H), 2.20−2.02 (m, 3H), 1.98−0.99 (series of multiplets, 40H), 0.92−0.85 (m, 9H), 0.65 (s, 3H); ¹³C NMR (CDCl₃, 75 MHz) δ 80.60, 79.74, 76.05, 64.97, 64.40, 64.28, 50.79, 50.25, 49.00, 48.90, 48.71, 46.47, 46.34, 42.65, 41.96, 39.80, 35.77, 35.41, 35.27, 35.05, 33.73, 31.96, 30.25, 29.76, 29.74, 29.67, 29.39, 29.05, 27.84, 27.61, 27.55, 26.70, 23.50, 23.00, 22.82, 22.79, 18.06, 14.23, 12.54; HRFAB-MS (thioglycerol+Na⁺ matrix) m/e: ([M+H]⁺) 755.6012 (100%), calcd. 755.6024. The triazide (0.18 g, 0.239 mmol) was dissolved in THF (10 mL) and EtOH (10 mL). Lindlar catalyst (44 mg) was added, and the suspension was shaken under H₂ (50 psi) for 12 hr. After removal of the solvent in vacuo, the residue was purified by silica gel chromatography (CH₂Cl₂/MeOH/28% NH₃.H₂O 10:5:1, then 10:5:1.5). To the product, 1 M HCl (2 mL) and the resulting clear solution was extracted with Et₂O (2×10 mL). 20% NaOH solution was added until the solution became strongly basic. CH₂Cl₂ (20 mL, 2×10 mL) was used to extract the basic solution. The combined extracts were dried over anhydrous Na₂SO₄, and removal of solvent in vacuo gave the desired product (0.114 g, 68% yield) as a clear oil. ¹H NMR (about 20% CDCl₃ in CD₃OD, 500 MHz) δ 4.79 (bs, 7H), 3.74−3.70 (m, 1H), 3.66−3.61 (m, 1H), 3.56−3.51 (m, 3H), 3.31−3.29 (m, 2H), 3.16−3.09 (m, 2H), 2.88−2.72 (m, 6H), 2.59−2.51 (m, 4H), 2.18−2.07 (m, 3H), 1.97−1.66 (series of multiplets, 14H), 1.62−0.97 (series of multiplets, 25H), 0.95 (d, J=6.3 Hz, 3H), 0.93 (s, 3H), 0.89 (t, J=6.8 Hz, 3H), 0.70 (s, 3H); ¹³C NMR (about 20% CDCl₃ in CD₃OD, 75 MHz) δ 81.82, 80.63, 77.23, 67.85, 67.19, 51.20, 50.69, 47.82, 47.24, 43.92, 43.01, 41.30, 40.80, 40.68, 40.22, 36.74, 36.38, 36.20, 35.87, 34.66, 34.15, 33.87, 32.90, 30.54, 30.39, 30.30, 29.64, 29.03, 28.59, 28.41, 26.96, 24.37, 23.65, 23.48, 18.75, 14.63, 13.09; HRFAB-MS (thioglycerol+Na⁺ matrix) m/e: ([M+H]⁺) 677.6309 (46.6%), calcd. 677.6309.

Compound CSA-46: Compound CSA-46 was prepared using the methods of CSA-13, substituting 7-deoxycholic steroid backbone precursor in place of cholic acid.

Compound 134: Compound CSA-13 (0.08 g, 0.12 mmol) was dissolved in CHCl₃ (5 mL) and MeOH (5 mL), aminoiminosulfonic acid (0.045 g, 0.36 mmol) was added, and the suspension was stirred for 12 hr. The solvent was removed in vacuo, and the residue was dissolved in 1 M HCl (6 mL) and H₂O (10 mL). The solution was washed with Et₂O (3×5 mL), and 20% NaOH solution was then added dropwise until the solution became strongly basic. The basic mixture was extracted with CH₂Cl₂ (4×5 mL). The combined extracts were dried over anhydrous Na₂SO₄ and concentrated in vacuo to give the desired product (0.087 g, 91% yield) as a white glass. ¹H NMR (about 20% CDCl₃ in CD₃OD, 500 MHz) δ 4.96 (bs, 13H), 3.74−3.68 (m, 1H), 3.65−3.50 (m, 4H), 3.38−3.18 (series of multiplets, 10H), 2.60−2.50 (m, 4H), 2.15−1.99 (m, 3H), 1.88−1.72 (m, 14H), 1.60−0.99 (series of multiplets, 25H), 0.94 (bs, 6H), 0.89 (t, J=6.6 Hz, 3H), 0.71 (s, 3H); ¹³C NMR (about 20% CDCl₃ in CD₃OD, 75 MHz) δ 159.00, 158.87, 158.72, 81.68, 79.93, 76.95, 66.59, 65.93, 65.45, 50.82, 50.40, 47.64, 46.94, 43.67, 42.27, 40.18, 39.25, 36.19, 35.66, 35.40, 34.21, 32.45, 30.51, 30.26, 30.18, 30.10, 29.86, 29.35, 28.71, 28.15, 28.00, 26.87, 23.94, 23.44, 23.23, 23.12, 18.61, 14.42, 12.98; HRFAB-MS (thioglycerol+Na⁺ matrix) m/e: ([M+H]⁺) 803.6958 (18.4%), calcd. 803.6953.

Compound CSA-15: The mesylate derived from 23 (0.092 g, 0.128 mmol) was dissolved in DMSO (2 mL) followed by the addition of NaN₃ (0.0167 g, 0.256 mmol). The suspension was heated to 70° C. for 12 hr. H₂O (20 mL) was added to the cooled suspension, and the mixture was extracted with EtOAc/hexanes (1:1) (20 mL, 3×10 mL). The combined extracts were washed with brine (30 mL), dried over anhydrous Na₂SO₄, and concentrated in vacuo to give the product (0.081 g, 95% yield) as a pale yellow oil. ¹H NMR (CDCl₃, 300 MHz) δ 3.69−3.36 (m, 11H), 3.25−3.02 (m, 6H), 2.20−2.02 (m, 3H), 1.97−1.60 (m, 15H), 1.55−0.98 (m, 13H), 0.92 (d, J=6.3 Hz. 3H), 0.89 (s, 3H), 0.66 (s, 3H); ¹³C NMR (CDCl₃, 75 MHz) δ 80.59, 79.77, 76.03, 65.01, 64.46, 64.30, 52.12, 48.99, 48.95, 48.76, 46.44, 46.42, 42.70, 41.99, 39.82, 35.56, 35.44, 35.31, 35.09, 33.09, 29.79, 29.77, 29.71, 29.08, 27.88, 27.78, 27.66, 25.65, 23.53, 23.03, 22.85, 18.00, 12.58; HRFAB-MS (thioglycerol+Na⁺ matrix) m/e: ([M+Na]⁺) 691.4512 (100%), calcd. 691.4496. The tetraazide (0.081 g, 0.12 mmol) was dissolved in THF (5 mL) and EtOH (10 mL). Lindlar catalyst (30 mg) was added, and the suspension was shaken under H₂ (50 psi) for 12 hr. After removal of the solvent in vacuo, the residue was purified by silica gel chromatography (CH₂Cl₂/MeOH/28% NH₃.H₂O 5:3:1, then 2:2:1). To the product, 1M HCl (2 mL) was added, and the resulting solution was washed with Et₂O (2×10 mL). 20% NaOH solution was added to the aqueous phase until the solution became strongly basic. CH₂Cl₂ (10 mL, 2×5 mL) was used to extract the basic solution. The combined extracts were dried over anhydrous Na₂SO₄, and concentration in vacuo gave the desired product (0.044 g, 64% yield) as a colorless oil. ¹H NMR (about 20% CDCl₃ in CD₃OD, 500 MHz) δ 4.79 (bs, 8H), 3.74−3.70 (m, 1H), 3.66−3.62 (m, 1H), 3.56−3.52 (m, 3H), 3.31−3.27 (m, 2H), 3.16−3.10 (m, 2H), 2.82−2.70 (m, 6H), 2.64−2.54 (m, 2H), 2.19−2.07 (m, 3H), 1.99−1.66 (series of multiplets, 14H), 1.58−0.96 (series of multiplets, 13H), 0.96 (d, J=6.6 Hz, 3H), 0.93 (s, 3H), 0.70 (s, 3H); ¹³C NMR (about 20% CDCl₃ in CD₃OD, 75 MHz) δ 81.96, 90.76, 77.33, 67.92, 67.26, 47.84, 47.33, 44.04, 43.24, 43.15, 41.40, 40.91, 40.78, 40.29, 36.82, 36.48, 36.28, 35.96, 34.39, 34.11, 30.59, 29.69, 29.13, 28.68, 28.64, 24.43, 23.69, 23.48, 18.77, 13.06; HRFAB-MS (thioglycerol+Na⁺ matrix) m/e: ([M+H]⁺) 565.5041 (100%), calcd. 565.5057.

### Example 12

This example includes a description of one or more exemplary synthestic procedures for obtaining Compounds 203a-b, 207a-c, 209a-c, 210a-b and CSA-31.

Compounds 203a-b, 207a-c, 208a-c, 209a-c, and 210a-b: BOC-glycine was reacted with DCC, DMAP and cholic acid derivative 201 (Scheme 11) to give triester 202a in good yield. A similar reaction incorporating BOC-p-alanine was also successful, giving 202b. Deprotection of 202a and 202b with HCl in dioxane, followed by purification (SiO₂ chromatography with a CH₂Cl₂ MeOH/NH₄OH eluent), gave triesters 203a and 203b in good yield.

Triamides of glycine and β-alanine (207a and 207b, respectively) were formed using the same reaction conditions (Scheme 12). Triamides with α-branched amino acids could also be formed. For example, under the conditions described, a triamide with bis-BOC-lysine side chains was formed (compound 207c). The C24 esters of 207a-c were hydrolyzed with LiOH in THF and methanol to give alcohols 208a-c. Deprotection using HCl in dioxane (208a-c) gave triamides 209a-c in good yield. In addition, alcohols 208a and 208b were mesylated and reacted with benzylmethyl amine. Deprotection of the resulting compounds with HCl in dioxane gave triamides 210a and 210b (Scheme 12). Compound CSA-31 was prepared by analogy to compounds 210a and 210b.

### Example 13

This example includes a description of one or more exemplary synthestic procedures for obtaining Compounds 302, 312-321, 324-326, 328-331 and 341-343.

Compound 302: Compound 308 (5β-cholanic acid 3,7,12-trione methyl ester) was prepared from methyl cholate and pyridinium dichromate in near quantitative yield from methyl cholate. Compound 308 can also be prepared as described in Pearson et al., J. Chem. Soc. Perkins Trans. l 1985, 267; Mitra et al., J. Org. Chem. 1968, 33, 175; and Takeda et al., J. Biochem. (Tokyo) 1959, 46, 1313. Compound 308 was treated with hydroxyl amine hydrochloride and sodium acetate in refluxing ethanol for 12 hr (as described in Hsieh et al., Bioorg. Med. Chem. 1995, 3, 823), giving 309 in 97% yield.

A 250 ml three neck flask was charged with glyme (100 ml); to this was added 309 (1.00 g, 2.16 mmol) and sodium borohydride (2.11 g, 55.7 mmol). TiCl₄ (4.0 mL, 36.4 mmol) was added to the mixture slowly under nitrogen at 0° C. The resulting green mixture was stirred at room temperature for 24 hours and then refluxed for another 12 h. The flask was cooled in an ice bath, and ammonium hydroxide (100 mL) was added. The resulting mixture was stirred for 6 hours at room temperature. Conc. HCl (60 mL) was added slowly, and the acidic mixture was stirred for 8 hours. The resulting suspension was made alkaline by adding solid KOH. The suspension was filtered and the solids were washed with MeOH. The combined filtrate and washings were combined and concentrated in vacuo. The resulting solid was suspended in 6% aqueous KOH (100 mL) and extracted with CH₂Cl₂ (4×75 mL). The combined extracts were dried over Na₂SO₄ and solvent was removed in vacuo to give 1.14 g of a white solid. The mixture was chromatographed on silica gel (CH₂Cl₂/MeOH/NH₄OH 12:6:1) giving 302 (0.282 g, 33% yield), 3 (0.066 g, 8% yield), 4 (0.118 g, 14% yield).

Compound 302: m.p. 200-202° C.; ¹H NMR (about 10% CDCl₃ in CD₃OD, 300 MHz) δ 4.81 (bs, 7H), 3.57−3.49 (m, 2H), 3.14 (t, J=3.2 Hz, 1H), 2.97 (bs, 1H), 2.55−2.50 (m, 1H), 2.15−2.10 (m, 1H), 1.95−1.83 (m, 3H), 1.74−0.99 (series of multiplets, 20H), 1.01 (d, J=6.4 Hz, 3H), 0.95 (s, 3H), 0.79 (s, 3H); ¹³C NMR (10% CDCl₃ in CD₃OD, 75 MHz) δ 63.28, 55.01, 52.39, 49.20, 48.69, 47.00, 43.24, 42.77, 41.03, 40.27, 36.82, 36.35, 35.75, 35.12, 32.77, 31.36, 30.10, 28.54, 27.88, 26.96, 24.35, 23.38, 18.18, 14.23, HRFAB-MS (thioglycerol+Na⁺ matrix) m/e; ([M+H]⁺) 392.3627 (100%); calcd. 392.3641.

Octanyl cholate (328): Cholic acid (3.14 g, 7.43 mmol) and 10-camphorsulfonic acid (0.52 g, 2.23 mmol) were dissolved in octanol (3.5 mL, 23.44 mmol). The solution was warmed to 40-50° C. in oil bath under vacuum (about 13 mm/Hg). After 14 h, the remaining octanol was evaporated under high vacuum. The crude product was purified via chromatography (silica gel, 5% MeOH in CH₂Cl₂) to afford the desired product (2.81 g, 73% yield) as a white powder. ¹H NMR (CDCl₃, 500 MHz) δ 4.06 (t, J=6.7 Hz, 2H), 3.98 (s, 1H), 3.86 (s, 1H), 3.48−3.44 (m, 1H), 2.41−2.34 (m, 1H), 2.28−2.18 (m, 3H), 1.98−1.28 (series of multiplets, 35H), 0.99 (d, J=3.3 Hz, 3H), 0.90 (s, 3H), 0.89 (t, J=7 Hz, 3H), 0.69 (s, 3H); ¹³C NMR (CDCl₃, 75 MHz) δ 154.38, 73.18, 72.14, 68.63, 56.07, 50.02, 49.32, 47.07, 46.74, 41.96, 41.67, 39.84, 39.76, 35.66, 35.45, 34.95, 34.86, 34.15, 32.97, 32.91, 31.65, 31.11, 30.68, 28.39, 27.78, 26.66, 26.52, 25.82, 25.70, 25.54, 25.15, 24.95, 23.45, 22.69, 17.77, 12.71; HRFAB-MS (thioglycerol+Na⁺ matrix) m/e: ([M+Na]⁺) 543.4015 (100%), calcd. 543.4026.

Representative synthesis of compounds 329-331: Octanyl cholate (328) (0.266 g, 0.511 mmol), N-t-Boc-glycine (0.403 g, 2.298 mmol), DCC (0.474 g, 2.298 mmol) and DMAP (0.0624 g, 0.051 mmol) were mixed in CH₂Cl₂ (15 mL) for 3 h. The resulting white precipitate was removed by filtration. The filtrate was concentrated, and the product was purified by chromatography (silica gel, EtOAc/Hexane 1:2) to afford the desired product (0.481 g, 95% yield) as a white powder. Compound 329 ¹H NMR (CDCl₃, 300 MHz) δ 5.18 (br, 3H), 5.01 (s, 1H), 4.61 (m, 1H), 4.04 (t, J=6.5 Hz, 2H), 3.97−3.88 (series of multiplets, 6H), 2.39−2.15 (series of multiplets, 2H), 2.06−1.02 (series of multiplets, 35H), 1.46 (s, 18H), 1.45 (s, 9H), 0.93 (s, 3H), 0.88 (t, J=6.7 Hz, 3H), 0.81 (d, J=6 Hz, 3H), 0.74 (s, 3H); ¹³C NMR (CDCl₃, 75 MHz) 6174.26, 170.19, 169.9, 169.78, 155.87, 155.67, 79.95, 76.47, 75.167, 72.11, 64.55, 47.40, 45.28, 43.17, 42.86, 40.82, 37.94, 34.71, 34.63, 34.43, 31.86, 31.340, 31.20, 30.76, 29.29, 29.25, 28.80, 28.72, 28.42, 28.06, 27.96, 27.19, 26.81, 26.29, 26.012, 25.66, 22.87, 22.71, 22.57, 17.55, 14.18, 12.27; HRFAB-MS (thioglycerol+Na⁺ matrix) m/e: ([M+Na]⁺) 1014.6261 (100%), calcd. 1014.6242. Compound 330: ¹H NMR (CDCl₃, 500 MHz) δ 5.10 (s, 1H), 4.92 (d, J=2.44 Hz, 1H), 4.55 (m, 1H), 4.00 (t, J=6.8 Hz, 2H), 3.39−3.33 (series of multiplets, 6H), 2.595−2.467 (series of multiplets, 6H), 2.31−2.12 (series of multiplets, 2H), 2.01-1.00 (series of multiplets, 37H), 1.39 (s, 27H), 0.88 (s, 3H), 0.84 (t, J=6.8 Hz, 3H), 0.76 (d, J=6.3 Hz, 3H), 0.69 (s, 3H); ¹³C NMR (CDCl₃, 75 MHz) δ 174.16, 172.10, 171.78, 171.67, 155.95, 79.45, 75.67, 74.21, 71.10, 64.63, 47.79, 45.27, 43.52, 40.97, 37.92, 36.35, 35.14, 35.05, 34.90, 34.71, 34.46, 31.91, 31.45, 30.95, 29.35, 29.31, 28.96, 28.78, 28.56, 28.55, 27.22, 26.98, 26.269, 25.71, 23.00, 22.77, 22.64, 17.75, 14.24, 12.39; HRFAB-MS (thioglycerol+Na⁺ matrix) m/e: ([M+Na]⁺) 1056.6702 (100%), calcd. 1056.6712. Compound 331 ¹³C NMR (CDCl₃, 125 MHz) δ 174.00, 172.75, 172.41, 172.30, 156.03, 79.00, 75.28, 73.79, 70.77, 64.39, 47.43, 45.04, 43.21, 40.76, 40.00, 39.93, 37.78, 34.74, 34.62, 34.23, 32.19, 32.01, 31.70, 31.24, 30.77, 29.13, 29.10, 28.67, 38.58, 28.38, 25.86, 25.37, 22.56, 22.38, 17.51, 14.05, 12.13; HRFAB-MS (thioglycerol+Na⁺ matrix) m/e: ([M+Na]⁺) 1098.7181 (100%), calcd. 1098.7181.

Representative synthesis of compounds 341-343: To compound 329 (0.463 g, 0.467 mmol) was added HCl in dioxane (0.3 mL, 4.0 M). After stirring the mixture for 30 min, the excess HCl and solvent were removed in vacuo. The product was isolated, after chromatography (silica gel, CH₂Cl₂/MeOH/NH₃.H₂O 10:1.2:0.1) as a (0.271 g, 84%) pale oil. The trihydrochloride salt of 341 was prepared by addition of HCl in dioxane and evaporation of excess HCl and dioxane in vacuo giving a white powder. Compound 341: ¹H NMR (CDCl₃ with about 10% CD₃OD, 500 MHz) δ 5.16 (s, 1H), 4.99 (t, J=3.6 Hz, 1 H), 4.61 (m, 1H), 4.04 (t, J=6.8 Hz, 2H), 3.51−3.36 (m, 6H), 2.34−2.15 (m, 2H), 2.00−1.05 (series of multiplets, 40H), 0.93 (s, 3H), 0.88 (t, J=7.1 Hz, 3H), 0.80 (d, J=3.2 Hz, 3H), 0.74 (s, 3H); ¹³C NMR (CDCl₃ and about 10% CD₃OD, 75 MHz) δ 174.32, 173.92, 173.81, 76.08, 74.67, 71.61, 64.73, 47.64, 45.39, 44.41, 43.49, 40.97, 37.99, 34.99, 34.77, 34.71, 34.52, 31.96, 31.54, 31.35, 30.96, 29.39, 29.36, 29.02, 28.82, 27.32, 27.11, 26.11, 25.83, 23.01, 22.82, 22.69, 17.79, 14.28, 12.41; HRFAB-MS (thioglycerol+Na⁺ matrix) m/e: ([M+Na]⁺) 714.4651 (100%), calcd. 714.4669. Compound 342: ¹H NMR (CDCl₃ and about 10% CD₃OD, 300 MHz) δ 5.142 (s, 1H), 4.96 (d, J=2.7 Hz, 1H), 4.60, (m, 1H), 4.04 (t, J=6.6 Hz, 2H), 3.07−2.95 (series of multiplets, 6H), 2.56−2.43 (series of multiplets, 6H), 2.38−2.13 (series of multiplets, 2 H), 2.07−1.02 (series of multiplets, 36H), 0.92 (s, 3H), 0.88 (t, J=6.6 Hz, 3H), 0.82 (d, J=6.6 Hz, 3H), 0.73 (s, 3H); ¹³C NMR (CDCl₃ and CD₃OD, 75 MHz) δ 174.29, 172.29, 171.98, 171.92, 75.52, 74.09, 70.98, 64.67, 47.78, 45.26, 43.52, 40.98, 38.73, 38.62, 38.35, 38.07, 38.03, 37.99, 35.01, 34.81, 34.77, 34.49, 31.92, 31.50, 31.40, 30.99, 29.36, 29.33, 28.93, 28.80, 27.43, 26.96, 26.08, 25.56, 23.07, 22.79, 22.62, 17.73, 14.25, 12.34; HRFAB-MS (thioglycerol+Na⁺ matrix) m/e: ([M+Na]⁺) 714.4651 (100%), calcd. 714.4669. Compound 343: ¹H NMR (CDCl₃ and CD₃OD, 500 MHz) δ 5.12 (s, 1H) 4.93 (s, 1H), 4.59 (m, 1H), 4.04 (t, J=7 Hz, 2H), 2.79−2.69 (series of multiplets, 6H), 2.4621−2.2999 (series of multiplets, 6H), 2.2033−1.0854 (series of multiplets, 42H), 0.94 (s, 2H), 0.91 (s, 1H), 0.88 (t, J=7 Hz, 3H), 0.82 (d, J=6.4 Hz, 3H), 0.75 (s, 3H); ¹³C NMR (CDCl₃ and CD₃OD, 75 MHz) δ 174.70, 171.97, 171.86, 171.75, 76.10, 74.55, 71.56, 64.85, 47.96, 45.31, 43.37, 40.87, 38.09, 34.86, 34.80, 34.73, 34.46, 32.84, 32.62, 32.27, 31.87, 31.75, 31.42, 31.08, 29.31, 29.28, 29.26, 28.78, 28.73, 27.38, 26.91, 26.05, 25.37, 23.24, 23.15, 22.95, 22.74, 22.71, 22.43, 17.78, 14.11, 12.28; HRFAB-MS (thioglycerol+Na⁺ matrix) m/e: ([M+Na]⁺) 798.5624 (100%), calcd. 798.5609.

Benzyl cholate (312): Cholic acid (4.33 g, 10.62 mmol) and 10-caphorsulfonic acid (0.493 g, 2.21 mmol) were dissolved in benzyl alcohol (1.97 mL, 19.3 mmol). The suspension was heated to 50° C. in oil bath and stirred under vacuum (about 13 mm/Hg) for 16 h. Excess benzyl alcohol was removed in vacuo, and the crude product was chromatographed (silica gel, 5% MeOH in CH₂Cl₂) to give the desire product as a white powder (4.23 g, 81% yield). ¹H NMR (CDCl₃, 500 MHz) δ 7.34−7.33 (m, 5H), 5.10 (d, J=1.5 Hz, 2H), 3.92 (s, 1H), 3.81 (s, 1H), 3.42 (s, 1H), 3.40 (br, m, 3H), 2.44−2.38 (m, 1H), 2.31−2.25 (m, 1H), 2.219 (t, J=12 Hz, 2H), 0.96 (d, J=5.5 Hz, 3H), 0.86 (s, 3H), 0.63 (s, 3H); ¹³C NMR (CDCl₃, 125 MHz) δ 174.25, 136.30, 128.66, 128.63, 128.32, 128.28, 128.24, 73.18, 71.98, 68.54, 66.18, 47.14, 46.56, 41.69, 39.65, 35.51, 35.37, 34.91, 34.84, 31.49, 31.08, 30.50, 28.31, 27.62, 26.47, 23.35, 22.65, 22.60, 17.42, 12.63, 12.57; HRFAB-MS (thioglycerol+Na⁺ matrix) m/e: ([M+Na]⁺) 521.3235 (100%), calcd. 521.3242.

Representative synthesis of compounds 313-315: Benzyl cholate (312) (0.248 g, 0.499 mmol), N-t-Boc-glycine (0.404 g, 2.30 mmol), DCC (0.338 g, 1.49 mmol) and DMAP (0.051 g, 0.399 mmol) were added to CH₂Cl₂ (15 mL), and the suspension was stirred for 16 h. The resulting white precipitate was removed by filtration, and the filtrate was concentrated. The product was obtained after chromatography (silica gel, EtOAc/Hexane 0.6:1) as a white powder (0.329 g, 68%). Compound 313: ¹H NMR (CDCl₃, 300 MHz) δ 7.34−7.33 (m, 5H), 5.16 (s, 1H), 5.08 (dd, J=22.5 Hz, 12.3 Hz, 4H), 5.00 (s, 1H), 4.60 (m, 1H), 4.04−3.81 (series of multiplets, 6H), 2.43−1.01 (series of multiplets, 25H), 1.46 (s, 9H), 1.44 (s, 18H), 0.92 (s, 3H), 0.797 (d, J=5.7 Hz, 3H), 0.69 (s, 1H); ¹³C NMR (CDCl₃, 75 MHz) δ 173.99, 170.25, 170.05, 169.85, 155.73, 136.19, 128.69, 128.45, 128.35, 80.06, 77.65, 77.23, 76.80, 76.53, 75.24, 72.19, 66.29, 47.46, 45.35, 43.24, 42.91, 40.89, 38.00, 34.79, 34.66, 34.49, 31.43, 31.25, 30.77, 28.88, 28.40, 27.23, 26.89, 25.74, 22.94, 22.65, 17.61, 12.32; FAB-MS (thioglycerol+Na⁺ matrix) m/e: ([M+Na]⁺) 992.5468 (100%), calcd. 992.5460.

Representative synthesis of compounds 316-318: Compound 313 (0.505 g, 0.520 mmol) and Pd (5 wt. % on active carbon, 0.111 g, 0.0521 mmol) were added to MeOH (5 mL). The suspension was stirred under H₂ (50 psi) for 20 hours. The solids were removed by filtration and the filtrate was concentrated. Purification of the product via chromatography (silica gel, 5% MeOH in CH₂Cl₂) gave a white powder (0.450 g, 98% yield). Compound 316: ¹H NMR (CDCl₃, 500 MHz) δ 5.20 (s, 1H), 5.12 (br, 2H), 4.92 (s, 1H), 4.55 (m, 1H), 3.98−3.83 (series of multiplets, 6H), 2.30−2.13 (series of multiplets, 2H), 1.96−0.98 (series of multiplets, 30H), 1.40 (s, 9H), 1.39 (s, 18H), 0.87 (s, 3H), 0.76 (d, J=6.3 Hz, 3H), 0.68 (s, 3H); ¹³C NMR (CDCl₃ 75 MHz) δ 174.11, 165.60, 165.41, 165.22, 151.28, 151.14, 75.48, 75.26, 71.81, 70.57, 67.50, 45.95, 42.58, 40.65, 38.52, 38.16, 36.17, 33.28, 30.01, 29.78, 26.71, 26.42, 25.95, 24.16, 23.78, 23.40, 23.31, 22.55, 22.16, 21.03, 18.23, 17.93, 12.91, 7.61; FAB-MS (thioglycerol+Na⁺ matrix) m/e: ([M+Na]⁺) 902.4997 (21%), calcd. 902.4990.

Representative synthesis of compounds 319-321: Compound 316 (0.375 g, 0.427 mmol), DCC (0.105 g, 0.512 mmol) and DMAP (0.062 g, 0.512 mmol) and N,N-dimethylethanolamine (0.09 ml, 0.896 mmol) were added to CH₂Cl₂ (15 mL). The mixture for 16 h, and solvent and excess N,N-dimethylethanolamine were removed in vacuo. The product was purified via chromatography (silica gel EtOAc/hexane/Et3 N, 12:10:0.6) giving a white powder (0.330 g, 82% yield). ¹H NMR (CDCl₃ and about 10% CD₃OD, 500 MHz) δ 5.18 (s, 1H), 5.00 (s, 1H), 4.19 (t, J=5.0 Hz, 2H), 3.92 (s, 3H), 3.81 (s, 3H), 2.62 (t, J=10 Hz, 2H), 2.30 (s, 6H), 1.47 (s, 9H), 1.47 (s, 1H), 1.45 (s, 1H), 2.12−1.05 (series of multiplets, 27H), 0.96 (s, 3H), 0.84 (d, J=10.5 Hz, 3H), 0.78 (s, 3H); ¹³C NMR (CDCl₃ and about 10% CD₃OD, 125 MHz) δ 174.19, 170.05, 169.87, 156.21, 79.36, 79.27, 76.06, 76.90, 71.80, 61.19, 57.04, 46.88, 44.87, 44.67, 44.53, 42.78, 42.15, 42.01, 40.43, 37.47, 34.32, 34.11, 33.92, 33.35, 33.25, 30.74, 30.56, 30.16, 28.40, 27.67, 27.62, 26.73, 26.19, 25.18, 25.10, 24.72, 24.49, 22.29, 21.81, 16.76, 11.56; FAB-MS (thioglycerol+Na⁺ matrix) m/e: ([M+Na]⁺) 973.5723 (100%), calcd. 973.5725. The white solid from the previous reaction (0.680 g, 0.714 mmol) and MeI (1 M in CH₂Cl₂, 1.5 mL) were stirred together for 2 h. The solvent and excess MeI were removed in vacuo giving a white solid (0.812 g about 100%). The product was carried on without further purification.

Representative synthesis of compounds 324-326: Compound 319 (0.812 g, 0.714 mmol) was dissolved in CH₂Cl₂ (5 mL) and trifluoroacetic acid (0.5 mL) was added. The mixture was stirred for 16 min. The solvent and excess acid were removed in vacuo, and the resulting oil was chromatographed (silica gel, CH₂Cl₂/MeOH/NH₃.H₂O 4:4:1) to give the desired product as a pale glass (0.437 g, 90% yield). Addition of HCl (2 M in ethyl ether, 2.5 mL) gave the trihydrochloride salt of 324 as a pale yellow powder. Compound 324: ¹H NMR (50% CDCl₃, 50% CD₃OD, 300 MHz) δ 5.43 (s, 1H), 5.24 (s, 1H), 4.84 (m, 1H), 4.66 (m, 2H), 4.16−3.96 (series of multiplets, 6H), 3.88 (m, 2H), 3.37 (s, 9H), 0.67 (s, 3H), 0.59 (d, J=6.3 Hz, 3H), 0.56 (s, 3H); ¹³C NMR (50% CDCl₃, 50% CD₃OD, 75 MHz) □ 173.47, 167.06, 167.01, 166.70, 78.01, 76.49, 73.78, 64.98, 57.67, 53.36, 47.49, 46.99, 45.61, 43.28, 40.83, 40.23, 40.10, 37.69, 34.80, 34.48, 34.28, 31.03, 30.63, 30.44, 28.94, 27.05, 26.56, 25.50, 22.53, 21.56, 16.95, 11.37; FAB-MS (thioglycerol+Na⁺ matrix) m/e: ([M−I]⁺) 665.4475 (85.6%), calcd 665.4489. Compounds 325 and 326 proved too unstable to chromatograph using the basic eluent used for the purification of 324. Consequently, 325 and 326 were prepared by deprotection of 320 and 321 using HCl (2 M in diethyl ether), followed by tituration with ethyl acetate. The compounds were then used without further purification. ¹H NMR spectroscopy indicated that compounds 325 and 326 were >95% pure. Compound 325: ¹H NMR (50% CDCl₃, 50% CD₃OD, 500 MHz) δ 5.21 (s, 1H), 5.02 (d, J=4 Hz, 1H), 4.64 (m, 1H), 4.53 (m, 2H), 3.74 (m, 2H), 3.31−3.01 (series of multiplets, 6H), 3.23 (s, 9H), 2.96−2.73 (series of multiples, 6H), 2.51−2.44 (m, 1H), 2.35−2.29 (m, 1H), 2.14−1.09 (series of multiplets, 26H), 0.99 (s, 3H), 0.85 (d, J=6.5 Hz, 3H), 0.80 (s, 3H); ¹³C NMR (50% CDCl₃, 50% CD₃OD, 125 MHz) δ 172.77, 169.88, 169.56, 169.50, 75.94, 74.44, 71.57, 64.31, 56.94, 52.92, 46.78, 44.59, 42.70, 40.21, 37.16, 34.80, 34.72, 34.66, 34.05, 34.00, 33.78, 33.62, 30.95, 30.91, 30.81, 30.41, 29.96, 29.81, 28.20, 26.37, 26.06, 24.74, 24.24, 22.04, 21.13, 16.54, 10.97; FAB-MS (thioglycerol+Na⁺ matrix) m/e: ([M−I]⁺) 707.4958 (25.6%), calcd 707.4958. Compound 326: ¹H NMR (50% CDCl₃, 50% CD₃OD, 500 MHz) δ 5.12 (s, 1H), 4.94 (d, J=2.5 Hz, 1H), 4.56 (m. 1H), 4.51 (t, J=2.3 Hz, 2H), 3.74 (m, 2H), 3.23 (s, 9H), 3.05−3.01 (m, 4H), 2.98 (t, J=7.5 Hz, 2H), 2.63−2.43 (series of multiplets, 6H), 2.31−2.24 (series of multiplets, 2H), 2.07−1.87 (series of multiplets, 12H), 1.17−1.05 (series of multiplets, 23H), 0.94 (s, 3H), 0.82 (d, J=6.0 Hz, 3H), 0.76 (s, 3H); ¹³C NMR (50% CDCl₃, 50% CD₃OD, 125 MHz) δ 171.87, 169.79, 169.59, 169.50, 76.12, 74.70, 71.65, 65.57, 65.08, 64.40, 57.68, 53.74, 52.78, 45.33, 43.54, 41.04, 39.12, 37.92, 43.85, 34.72, 34.56, 34.34, 32.30, 31.47, 31.27, 30.87, 30.58, 29.03, 27.053, 26.84, 25.51, 24.95, 24.91, 22.87, 22.82, 22.65, 21.93, 17.31, 11.81; FAB-MS (thioglycerol+Na⁺ matrix) m/e: ([M−I]⁺) 749.5432 (100%), calcd 749.5436.

### Example 14

This example includes data indicating the stability of Compounds 352-354 under acidic, neutral and basic conditions.

Compounds 352-354 were dissolved in 50 mM phosphate buffered water (pH 2.0, 7.0 or 12.0) at approximately 10 mM concentrations. The structures of compounds 352-354 are given in FIG. 9. Decomposition of the compounds was observed via HPLC (cyano-silica column, 0.15% TFA water-acetonitrile gradient elution). Table 15 shows the stabilities (half-lives) of compounds 352-354 in phosphate buffer at room temperature, pH 2.0, pH 7.0 and pH 12.0. These compounds were used since they contain a chromophore that facilitated monitoring of decomposition by absorption methods common in the HPLC apparatus used.

At low pH, the amines are expected to be protonated and the compounds showed relative stability. At higher pH, the amines were less strongly protonated and became involved in ester hydrolysis. The γ-aminobutyric acid-derived compound was especially susceptible to hydrolysis, presumably yielding pyrrolidone. In general, the compounds are believed to hydrolyse to give cholic acid, choline or octanol, and glycine, beta-alanine, or pyrrolidone, depending on the particular compound.

Decomposition through ester hydrolysis yielded compounds that were less polar and easily separable from the starting compounds. Initially, only one benezene-containing decomposition product was observed; at longer reaction times, two other decomposition products were observed which presumably corresponded to sequential ester hydrolysis.

### Example 15

This example includes a description of additional exemplary synthestic procedures for producing compounds of formula I. In one example, hydroxyl groups on cholic acid can be converted into amine groups as described in Hsieh et al. (Synthesis and DNA Binding Properties of C3-, C12-, and C24-Substituted Amino-Steroids Derived from Bile Acids, Bioorganic and Medicinal Chemistry, 1995, vol. 6, 823−838).

Compounds of formula I prepared as shown in the following Scheme.

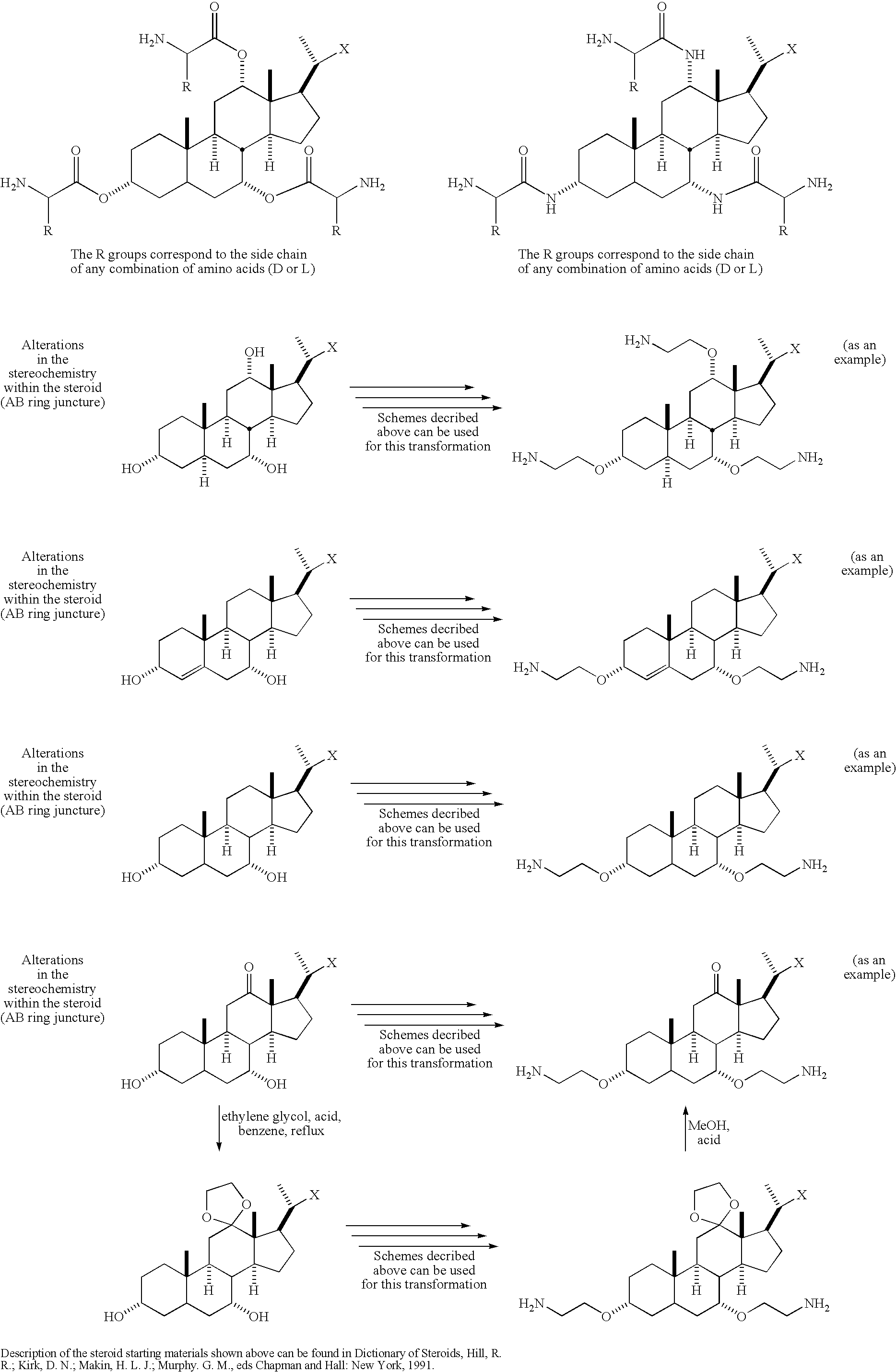

### Example 16

This example describes various materials and methods.

Cell Culture and Primary Cell Isolation: Peripheral blood mononuclear cells (PBMC) were isolated from adult blood using a ficoll-hypaque gradient. Monocytes and CD4+ T cells were isolated from PBMC using AutoMACS. DCs were generated by culturing CD14+ monocytes/ml in RPMI complete (10% fetal bovine serum (FBS), 2 mM L-glutamine, 100 U/ml of penicillin G, 100 μg/ml of streptomycin) medium supplemented with IL-4 (R&D Systems, 50 μg/ml) and GM-CS F (R&D Systems, 50 μg/ml) for 5 days and subsequently matured by addition of LPS (Sigma, 100 ng/ml) for 1-2 days. Mature DC production was assessed by staining cells with antibodies to CD14, CD83, CD86, and HLA-DR (all from BD Biosciences). Hut 78 T cells expressing CCR5 (Hut/CCR5) were prepared and maintained as previously described (Oswald-Richter et al., Eur. J. Immunol. 34:1705 (2004); Oswald-Richter et al., PLoS Biol. 2:E198 (2004)).

Virus production: Vesicular stomatitis virus glycoprotein (VSV-G)-pseudotyped replication-incompetent HIV particles (HDV-VSV-G) were generated by co-transfecting HEK-293T cells with an envelope negative proviral plasmid and a VSV-G envelope plasmid. Replication competent virus expressing the HIV envelope BaL that uses CCR5 as co-receptor (HIV-R5) was generated by transfecting HEK-293T cells with the NIA-3 proviral plasmid. All these viruses also contain EGFP (Clontech) in place of the nef gene. Supernatants were collected and infection was tittered on HUT cells to determine Infectious units (IFU) per ml.

HIV infection and cell viability assays: Virus was cultured in the presence of CSAs at various time points and concentrations with Hut or primary CD4⁺ T cells activated by cross-linking with plate-bound anti-CD3 antibody (OKT-3, ATCC) and soluble anti-CD28 antibody (BD Biosciences). The plates were first coated with anti-mouse IgG (10 g/ml, Caltag), followed by anti-CD3 antibody. Infection of T cells was analyzed through GFP expression after 3 days using a FACSCalibur™ four-color cytometer (BD Biosciences) and CELLQuest™ software (BD Biosciences). Aliquots of cells were removed at different time points post peptide treatment and incubated with propidium iodide (PI, Sigma, 25 μg/ml). Cells were analyzed by flow cytometry for PI exclusion as an indicator of viability. All data were normalized to control treated infection levels set at 100% for each data point.

DC mediated infection assays: Monocyte-derived DC was pulsed with replication-competent HIV-R5 at an MOI of 2. Virus-cell mixtures were centrifuged at 2000 rpm for 1 hour and cultured for 2 additional hours to allow DCs to efficiently capture virus. DCs were washed three times with complete RPMI medium to remove non cell-associated virus. CSAs were added to DC at different concentrations and incubated for 1 h. DCs were washed three times with complete RPMI medium and incubated with 1.5×10⁴ Hut/CCR5 cells for 3 days. Cells were harvested, fixed with 1% paraformaldehyde, and analyzed for expression of GFP by flow cytometry. In some studies, DC was incubated alone after CSA treatment for 24 h and assayed for viability using PI staining as described above.

HIV p24 assay: HIV-VSV-G was incubated with CSAs or control at different concentrations for 30 min in complete RPMI medium. The medium was then assayed for the presence of viral core protein p24 by ELISA. Plates were analyzed by microplate reader (Molecular Devices) at 405 nm absorbance. Total p24 was calculated using linear regression analysis from standards included on each plate.

### Example 17

This example describes HIV-VSV-G infectivity studies in the presence of various CSAs.

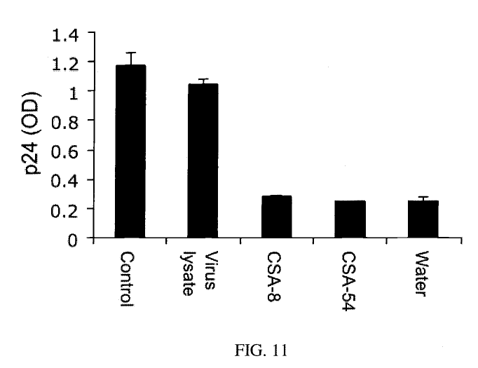

HIV-VSV-G (30,000 infectious units) was incubated alone or with 200 μM CSA-8, 50 μM CSA-54, positive control peptide (caerin 1.9 at 10 μM) or with water diluted in RPMI for 30 min in complete RPMI medium. The medium was then assayed for the presence of viral core protein p24 by ELISA. Plates were analyzed by microplate reader (Molecular Devices) at 405 nm absorbance. Data are representative of four independent studies (FIG. 11).

### Example 18

This example describes viability studies of various cells using flow cytometry.

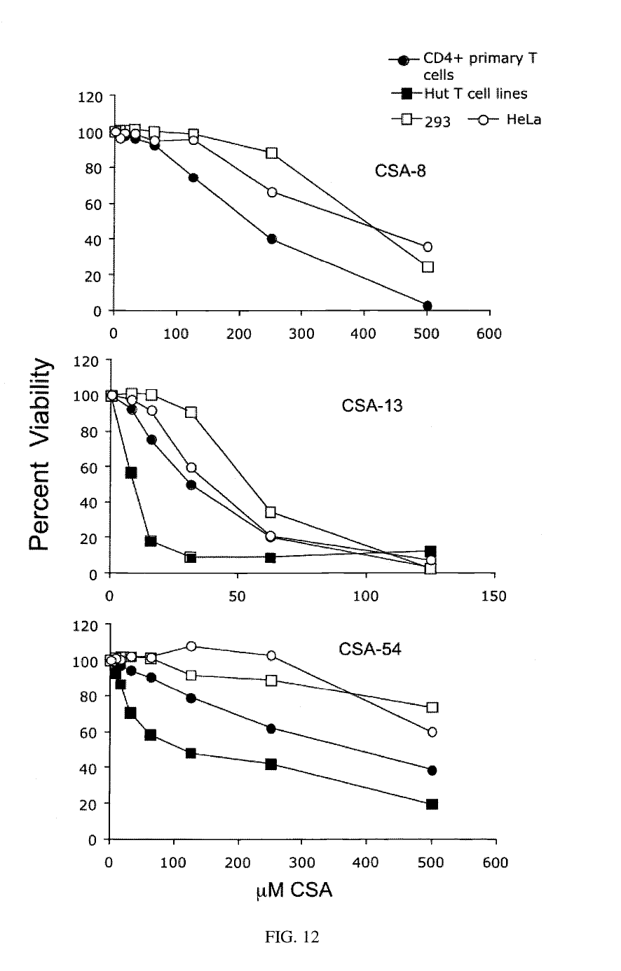

CSA's were incubated with 5×10⁵ Hut cells (closed squares), activated primary CD4+ T cells (closed circles), HEK-293T cells (open squares) or HeLa cells (open circles) for 1 h, removed from the culture, stained with PI, and analyzed for viability by flow cytometry (FIG. 12).

### Example 19

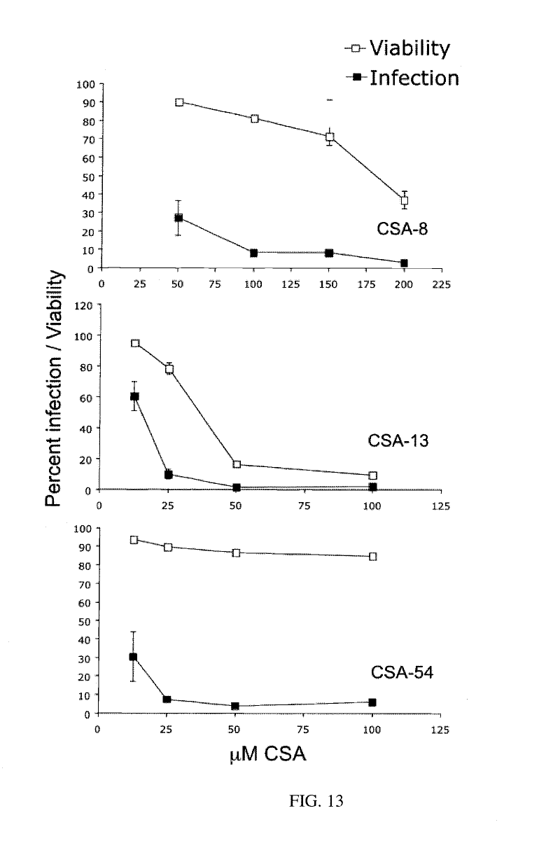

This example describes viability studies of infectious HIV-VSV-G using flow cytometry. CSA's were incubated with HIV-VSV-G (2×10⁵ infectious units) and 1×10⁵ Hut cells for 5 min then diluted 4-fold with complete RPMI medium and incubated at 37° C. for 3 days. Cells were harvested and analyzed for GFP expression (closed squares). Data are normalized to infection following water treatment and are presented as the mean of three replicate samples from one representative study with error bars indicating standard deviation (FIG. 13). At 24 hours post infection 1.5×10⁴ T cells were removed from the culture, stained with PI, and analyzed for viability by flow cytometry (open squares).

What is claimed:

1. A method for providing a subject with protection against human immunodeficiency virus (HIV) infection or pathogenesis, comprising administering a sufficient amount of cationic steroid antimicrobial (CSA) to provide the subject with protection against human immunodeficiency virus (HIV) infection or pathogenesis.

2. A method for treating a subject in need of treatment for human immunodeficiency virus (HIV) infection or pathogenesis, comprising administering a sufficient amount of cationic steroid antimicrobial (CSA) to treat the subject for the human immunodeficiency virus (HIV) infection or pathogenesis.

3. A method for decreasing susceptibility of a subject to a human immunodeficiency virus (HIV) infection or pathogenesis, comprising administering a sufficient amount of cationic steroid antimicrobial (CSA) to decrease susceptibility of the subject to human immunodeficiency virus (HIV) infection or pathogenesis.

4. The method of any of claims 1 to 3, wherein the CSA is administered prior to, concurrently with, or following infection of the subject with or exposure to or contact of the subject with HIV.

5. The method of any of claims 1 to 3, wherein the CSA is administered prior to, concurrently with, or following development of a symptom of acute or chronic HIV infection.

6. The method of any of claims 1 to 3, wherein the HIV comprises a drug resistant HIV type, group, subtype or isolate.

7. The method of any of claims 1 to 3, wherein the HIV comprises HIV−1 or HIV−2.

8. The method of any of claims 1 to 3, wherein the HIV−1 comprises a Group M, N or O group.

9. The method of any of claims 1 to 3, wherein the HIV−1 comprises an A, B, A/B, A/E, A/G, C, D, F, G, H, J or K subtype, or a mixture thereof.

10. The method of any of claims 1 to 3, wherein the CSA is selected from CSA-7, CSA-8, CSA-10, CSA-11, CSA-13, CSA-15, CSA-17, CSA-21, CSA-25, CSA-26, CSA-31, CSA-46, CSA-54 and CSA-59, as set forth in FIG. 10.

11. The method of any of claims 1 to 3, wherein the CSA does not have a charged group at position C24.

12. The method of any of claims 1 to 3, wherein the CSA has a hydrophobic moiety at position C24.

13. The method of claim 12, wherein the hydrophobic moiety at position C24 comprises a lipid.

14. The method of any of claims 1 to 3, wherein the CSA has a charged group at position C7.

15. The method of any of claims 1 to 3, wherein the CSA comprises a multimer.

16. The method of any of claims 1 to 3, wherein the CSA multimer comprises a dimer, trimer, or tetramer.

17. The method of any of claims 1 to 3, wherein the CSA has a shorter tether length between the steroid scaffold and the amine groups at positions C3, C7 and C12, relative to the tether length of CSA-7, CSA-8, CSA-10, CSA-11, CSA-13, CSA-15, CSA-17, CSA-21, CSA-25, CSA-26, CSA-31, CSA-46, CSA-54 or CSA-59, as set forth in FIG. 10.

18. The method of any of claims 1 to 3, wherein the CSA comprises a pharmaceutically acceptable carrier or excipient.

19. The method of any of claims 1 to 3, wherein the CSA comprises a sterile formulation.

20. The method of any of claims 1 to 3, wherein the CSA comprises a composition comprising one or more additional biologically active ingredients.

21. The method of any of claims 1 to 20, wherein the subject has a CD4+ T cell count less than 500 cells/microliter blood, less than 200 cells/microliter blood, or the percentage of CD4+ T cells in the subject is less than 15% of all lymphocytes.

22. The method of any of claims 1 to 20, wherein the subject is provided with partial or complete protection against HIV infection or pathogenesis, or a symptom caused by HIV infection or pathogenesis.

23. The method of any of claims 1 to 20, wherein the method reduces, decreases, inhibits, ameliorates or prevents onset, severity, duration, progression, frequency or probability of one or more symptoms associated with or caused by HIV infection or pathogenesis in a subject.

24. The method of claim 23, wherein the symptom is selected from: fever, fatigue, headache, sore throat, swollen lymph nodes, weight loss, diarrhea, rash, boils, warts, thrush, shingles, chronic or acute pelvic inflammatory disease (PID), dry cough, shortness of breath, bruising, bleeding, numbness or paralysis, muscle weakness, an opportunistic disorder, nerve damage, encephalopathy, dementia and death.

25. The method of claim 24, wherein the opportunistic disorder is selected from bacterial, viral, fungal and parasitic infection.

26. The method of claim 24, wherein the opportunistic disorder is selected from: Candidiasis of bronchi, trachea, lungs or esophagus, cervical cancer, Coccidioidomycosis, Cryptococcosis, Cryptosporidiosis, Bacillary Angiomatosis, Cytomegalovirus (CMV), Cytomegalovirus retinitis, Herpes virus, Hepatitis virus, papilloma virus, Histoplasmosis, Isosporiasis, Kaposi's sarcoma, Burkitt's lymphoma, immunoblastic lymphoma, Mycobacterium avium, Mycobacterium tuberculosis, Pneumocystis carinii, Pneumonia, progressive multifocal leukoencephalopathy (PML), Salmonelosis, Toxoplasmosis, Wasting syndrome and Lymphoid interstitial pneumonia/pulmonary lymphoid type.

27. The method of any of claims 1 to 20, wherein the method prevents or inhibits a worsening or progression of HIV infection or one or more symptoms associated with HIV infection or pathogenesis.

28. The method of any of claims 1 to 20, wherein the method stabilizes the HIV infection or one or more symptoms associated with the HIV infection or pathogenesis.

29. The method of any of claims 1 to 20, wherein the method reduces or decreases HIV titer, viral load, viral replication, viral proliferation or a viral protein, or inhibits or prevents increases in HIV titer, viral load, viral replication, viral proliferation or a viral protein.

30. The method of any of claims 1 to 20, wherein the method reduces or decreases susceptibility of the subject to HIV infection or one or more symptoms associated with HIV infection or pathogenesis.

31. The method of any of claims 1 to 20, wherein the method increases or stabilizes numbers of CD4+ T cells in the subject.

32. The method of any of claims 1 to 20, wherein the subject has not been infected with or exposed to HIV.

33. The method of any of claims 1 to 20, wherein the subject has been vaccinated against HIV.

34. The method of any of claims 1 to 20, wherein the subject has been infected with or exposed to HIV.

35. The method of any of claims 1 to 20, wherein the subject has been diagnosed as HIV+.

36. The method of any of claims 1 to 20, wherein the subject is immunocompromised.

37. The method of any of claims 1 to 20, wherein the subject is a candidate for or has received an immunosuppressant treatment.

38. The method of any of claims 1 to 20, wherein the subject is a candidate for or has received a tissue or organ transplant.

39. The method of any of claims 1 to 20, wherein the subject is a newborn, infant, toddler or child.

40. The method of any of claims 1 to 20, wherein the subject is 50 years or older.

41. The method of any of claims 1 to 20, further comprising administering to the subject an additional CSA or other treatment.

42. The method of claim 41, wherein the treatment is for HIV, a side effect of an HIV treatment or for an opportunistic disorder caused by an HIV infection or an HIV treatment.

43. The method of claim 41, wherein the treatment comprises a protease inhibitor, a reverse transcriptase inhibitor, a virus fusion inhibitor or a virus entry inhibitor.

44. The method of claim 41, wherein the treatment comprises administering: AK602, AMD070, APV, ATV, ATZ, AVX754, AZT, Abacavir, Acyclovir, Adefovir dipivoxil, Adriamycin, Agenerase, Aldesleukin, Alovudine, AmBisome, Amdoxovir, Amphocin, Amphotec, Amphotericin B, Ampligen, Amprenavir, Androderm, Androgel, Aptivus, Atazanavir, Azithromycin, BMS-488043, Bactrim, Baraclude, Biaxin, BufferGel, C31G, CD4-IgG2, CPV, CS, Calanolide A, Capravirine, Carbopol 974P, Carrageenan, Carraguard, Cellulose sulfate, Clarithromycin, Combivir, Copegus, Cotrimoxazole, Crixivan, Cyanovirin-N, Cytovene, DAPD, DLV, DPC 817, DS, Delavirdine, Depo-Testosterone, Dextran sulfate, Didanosine, Diflucan, Doxil, Doxorubicin, Dronabinol, EFV, Efavirenz, Elvucitabine, Emtricitabine, Emtriva, Enfuvirtide, Entecavir, Epivir, Epoetin alfa, Epogen, Epzicom, Etopophos (phosphate salt), Etoposide, Etravirine, Fluconazole, Fortovase, Fosamprenavir, Fungizone, Fuzeon, GSK-873,140 (aplaviroc), GW433908, Gammar-P, Ganciclovir, Growth hormone, Human growth hormone, HEC, Hepsera, Hivid, Hydroxyethyl cellulose, IDV, IGIV, Interleukin-2 (IL-2), INH, Immune Globulin, Indinavir, Interferon alfa-2, Intron A (2b), Invirase, Isoniazid, Itraconazole, KP-1461, Kaletra, L-000870810, LPV/RTV, Lamivudine, Lexiva, Marinol, Megace, Megestrol, Mycobutin, NFV, NVP, Naphthalene 2-sulfonate polymer, Nebupent, Nelfinavir, Neutrexin, Nevirapine, New-Fill, Norvir, Nydrazid, Onxol, PA-457, PMPA, PRO 2000, PRO 542, Paclitaxel, Paxene, Pegasys (2a), Pentamidine, Peptide T, Poly(I)-Poly(C12U), Poly-L-lactic acid, Polygam S/D, Procrit, Proleukin, RCV, RTV, RVT, Racivir, Rebetol, Rescriptor, Retrovir, Reverset, Reyataz, Ribavirin, Rifabutin, Rifadin, Rifampin, Rimactane, Ritonavir, Roferon-A (2a), SCH-C, SCH-D (vicriviroc), SQV, Saquinavir, Savvy, Sculptra, Septra, Serostim, Somatropin, Sporanox, Stavudine, Sulfamethoxazole, Sustanon, Sustiva, T-20, TDF, THC, TMC114, TMC125, TNX-355, Taxol, Tenofovir, Tenofovir disoproxil fumarate, Testosterone, Tipranavir, Toposar, Trimethoprim, Trimetrexate, Trizivir, Truvada, UC-781, UK-427,857 (maraviroc), Ushercell, Valcyte, Valganciclovir, Valproic acid, VePesid, Vicriviroc, Videx, Viracept, Viramune, Virazole, Viread, Vitrasert, ZDV, Zalcitabine, Zerit, Ziagen, Zidovudine, Zithromax, Zovirax, D4T, ddC, β-LFddC, P-LFd4C, DDI, f-APV, 3TC, or human erythropoietin (EPO).

45. The method of claim 41, wherein the treatment comprises a cytokine, chemokine, interferon or interleukin.

46. The method of claim 41, wherein the treatment is for a tumor or cancer.

47. The method of claim 46, wherein the tumor or cancer treatment comprises internal or external radiotherapy, surgical resection, hyperthermia, or a chemotherapeutic agent.

48. The method of claim 41, wherein the treatment comprises an antibody that binds to an HIV protein.

49. The method of claim 48, wherein the HIV protein is selected from: envelope protein gp160, gp120 or gp41, gag protein, pol protein, p7, p17, p24, tat, rev, nef, vif, vpr, vpu, reverse transcriptase, integrase, and protease.

50. The method of claim 48, wherein the antibody is human, humanized or chimeric.

51. The method of claim 48, wherein the antibody is monoclonal or polyclonal.

52. A method for increasing or stabilizing numbers of CD4+ T cells in an HIV+ subject, comprising administering a sufficient amount of cationic steroid antimicrobial (CSA) to increase or stabilize numbers of CD4+ T cells in the HIV+ subject.

53. A method for decreasing or inhibiting HIV infection of a cell in vitro or in vivo, comprising administering a composition comprising a sufficient amount of cationic steroid antimicrobial (CSA) to inhibit HIV infection of the cell.

54. The method of claim 53, wherein the cell is mammalian.

55. The method of claim 53, wherein the cell is human.

56. A method for providing a subject with protection against HIV infection or pathogenesis, comprising administering a sufficient amount of CSA-7, CSA-8, CSA-10, CSA-11, CSA-13, CSA-15, CSA-17, CSA-21, CSA-25, CSA-26, CSA-31, CSA-46, CSA-54 and CSA-59, as set forth in FIG. 10, to provide the subject with protection against the HIV infection or pathogenesis.

57. A method for treating a subject in need of treatment for an HIV infection or pathogenesis, comprising administering a sufficient amount of CSA-7, CSA-8, CSA-10, CSA-11, CSA-13, CSA-15, CSA-17, CSA-21, CSA-25, CSA-26, CSA-31, CSA-46, CSA-54 and CSA-59, as set forth in FIG. 10, to treat the subject for the HIV infection or pathogenesis.

58. A method for decreasing susceptibility of a subject to an HIV infection or pathogenesis, comprising administering a sufficient amount of CSA-7, CSA-8, CSA-10, CSA-11, CSA-13, CSA-15, CSA-17, CSA-21, CSA-25, CSA-26, CSA-31, CSA-46, CSA-54 and CSA-59, as set forth in FIG. 10, to decrease susceptibility of the subject to the HIV infection or pathogenesis.

59. A method for reducing, decreasing, inhibiting, ameliorating or preventing onset, severity, duration, progression, frequency or probability of one or more symptoms associated with HIV infection or pathogenesis in a subject, comprising administering a sufficient amount of CSA-7, CSA-8, CSA-10, CSA-11, CSA-13, CSA-15, CSA-17, CSA-21, CSA-25, CSA-26, CSA-31, CSA-46, CSA-54 and CSA-59, as set forth in FIG. 10, to decrease, inhibit, ameliorate or prevent onset, severity, duration, progression, frequency or probability of one or more symptoms associated with HIV infection or pathogenesis in the subject.

60. A kit, said kit comprising packaging material, a cationic steroid antimicrobial (CSA) and instructions, said instructions comprising administering said CSA to:
a) provide a subject with protection against an HIV infection or pathogenesis;
b) treat a subject for HIV infection or pathogenesis;
c) decrease susceptibility of a subject to HIV infection or pathogenesis; or
d) decrease, inhibit, ameliorate or prevent onset, severity, duration, progression, frequency or probability of one or more symptoms associated with HIV infection or pathogenesis.

61. A method for identifying a candidate agent for treating a subject for an HIV infection or pathogenesis, comprising:
a) providing a test agent, said test agent comprising a cationic steroid antimicrobial (CSA);
b) contacting said test agent with HIV and ascertaining whether the test agent inhibits HIV infection or pathogenesis, wherein a test agent identified as inhibiting HIV infection or pathogenesis is a candidate agent for treating a subject for HIV infection or pathogenesis.

62. A method for identifying a candidate agent for decreasing susceptibility of a subject to an HIV infection or pathogenesis, comprising:
a) providing a test agent, said test agent comprising a cationic steroid antimicrobial (CSA);
b) contacting said test agent with HIV and ascertaining whether the test agent inhibits HIV infection or pathogenesis, wherein a test agent identified as inhibiting HIV infection or pathogenesis is a candidate agent for decreasing susceptibility of a subject to an HIV infection or pathogenesis.

63. A method for identifying a candidate agent for decreasing, inhibiting, ameliorating or preventing onset, severity, duration, progression, frequency or probability of one or more symptoms associated with HIV infection or pathogenesis, comprising:
a) providing a test agent, said test agent comprising a cationic steroid antimicrobial (CSA);
b) administering said test agent to a subject infected with or exposed to HIV and ascertaining whether the test agent decreases, inhibits, ameliorates or prevents onset, severity, duration, progression, frequency or probability of one or more symptoms associated with HIV infection or pathogenesis, wherein a test agent identified is a candidate agent for decreasing, inhibiting, ameliorating or preventing onset, severity, duration, progression, frequency or probability of one or more symptoms associated with HIV infection or pathogenesis.

64. The method of claim 63, wherein the subject comprises a mammal.

65. The method of claim 63, wherein the subject comprises a primate.

66. The method of claims 64 or 65, wherein the mammal or primate comprises an animal model for HIV infection or pathogenesis.
